(19) United States
(12) Patent Application Publication
(10) Pub. No.: US 2025/0340867 A1
(43) Pub. Date: Nov. 6, 2025

(54) DNA ORIGAMI SUBUNITS AND THEIR USE FOR ENCAPSULATION OF FILAMENTOUS VIRUS PARTICLES

(71) Applicants: BRANDEIS UNIVERSITY, Waltham, MA (US); University of Massachusetts Amherst, Amherst, MA (US)

(72) Inventors: Seth FRADEN, Waltham, MA (US); Tijana IVANOVIC, Waltham, MA (US); Michael HAGAN, Waltham, MA (US); William Benjamin ROGERS, Waltham, MA (US); Greg GRASON, Waltham, MA (US); Daichi HAYAKAWA, Waltham, MA (US); Doug HALL, Waltham, MA (US)

(21) Appl. No.: 18/569,150

(22) PCT Filed: Aug. 29, 2022
(86) PCT No.: PCT/US2022/032822

§ 371 Date: Dec. 11, 2023

Related U.S. Application Data
(60) Provisional application No. 63/208,725, filed on Jun. 9, 2021.

(51) Int. Cl. C12N 15/11; A61K 31/711; A61P 31/12; C12N 7/00

(52) U.S. Cl. CPC C12N 15/11; A61K 31/711; A61P 31/12; C12N 7/00

(57) ABSTRACT

The present disclosure relates to three-dimensional nucleic acid origami nanostructures that are designed to allow for self-assembly of the nanostructures into a larger structure (e.g., cylindrical, icosahedral, etc.) about the surface of a virus particle, and their use in treatment methods.

This application claims the priority benefit of U.S. Provisional Patent Application Ser. No. 63/208,725, filed Jun. 9, 2021, which is hereby incorporated by reference in its entirety.

This invention was made with government support under MRSEC 1420382 awarded by the National Science Foundation. The government has certain rights in the invention.

### FIELD OF THE INVENTION

The present disclosure relates to DNA origami subunits and their use for encapsulation of filamentous virus particles.

### BACKGROUND OF THE INVENTION

For the majority of viral diseases, no effective treatment is available. Broadly applicable antiviral platform technologies do not exist.

Protein designers have previously succeeded in creating artificial macromolecular cages (Bale et al., “Accurate Design of Megadalton-Scale Two-Component Icosahedral Protein Complexes,” Science 353:389-394 (2016); King et al., “Accurate Design of Co-Assembling Multi-Component Protein Nanomaterials,” Nature 510:103-108 (2014); Lai et al., “Structure of a Designed Protein Cage that Self-Assembles into a Highly Porous Cube,” Nature Chemistry 6:1065-1071 (2014); Butterfield et al., “Evolution of a Designed Protein Assembly Encapsulating its Own RNA Genome,” Nature 552:415-420 (2017)). However, the designed protein-cages are much smaller than the vast majority of natural viruses and cannot be easily modified. DNA nanotechnology (Rothemund, “Folding DNA to Create Nanoscale Shapes and Patterns,” Nature 440:297-302 (2006); Douglas et al., “Self-Assembly of DNA into Nanoscale Three-Dimensional Shapes,” Nature 459:414-418 (2009); Castro et al., “A Primer to Scaffolded DNA Origami,” Nature Methods 8:221-229 (2011); Veneziano et al., “Designer Nanoscale DNA Assemblies Programmed from the Top Down,” Science 352:1534 (2016); Benson et al., “DNA Rendering of Polyhedral Meshes at the Nanoscale,” Nature 523:441-444 (2015); Dunn et al., “Guiding the Folding Pathway of DNA Origami,” Nature 525:82-86 (2015)) can create discrete objects with structurally well-defined 3D shapes (Bai et al., “Cryo-EM Structure of a 3D DNA-Origami Object,” PNAS 109:20012-20017 (2012); Funke et al., “Placing Molecules with Bohr Radius Resolution Using DNA Origami,” Nature Nanotechnology 11:47-52 (2016)), including higher-order objects (Iinuma et al., “Polyhedra Self-Assembled from DNA Tripods and Characterized with 3D DNA-PAINT,” Science 344:65-69 (2014); Jungmann et al., “DNA Origami-Based Nanoribbons: Assembly, Length Distribution, and Twist,” Nanotechnology 22:275301 (2011); Liu et al., “Crystalline Two-Dimensional DNA-Origami Arrays,” Angewandte Chemie 50:264-267 (2011); Suzuki et al., “Lipid-Bilayer-Assisted Two-Dimensional Self-Assembly of DNA Origami Nanostructures,” Nature Communications 6:8052 (2015); Ke et al., “DNA Brick Crystals with Prescribed Depths,” Nature Chemistry 6:994-1002 (2014)) with molecular masses exceeding one Gigadalton (Wagenbauer et al., “Gigadalton-Scale Shape-Programmable DNA Assemblies,” Nature 552:78-83 (2017)). However, these previous designs and the underlying concepts yield objects that are either too small, assemble with insufficient yields, do not match the shapes of viruses, or are too flexible or skeletal to be suitable for effectively trapping and occluding entire virus particles.

The present disclosure is directed to overcoming these and other deficiencies in the art.

### SUMMARY OF THE INVENTION

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a macromolecular icosahedral shell.

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a macromolecular cylindrical shell.

Another aspect of the present disclosure relates to a macromolecular cylindrical shell formed by self-assembly of a plurality of the three-dimensional DNA molecular structures described herein.

A further aspect of the present disclosure relates to a composition comprising a plurality of three-dimensional DNA molecular structures described herein in a carrier.

Another aspect of the present disclosure relates to a composition comprising a plurality of macromolecular cylindrical shells described herein in a carrier.

A further aspect of the present disclosure relates to a composition comprising a plurality of three-dimensional DNA molecular structures described herein and a plurality of macromolecular cylindrical shells as described herein in a carrier.

Another aspect of the present disclosure relates to a method of encapsulating a filamentous viral particle. This method involves providing a plurality of the three-dimensional DNA molecular structures described herein, and allowing said three-dimensional DNA molecular structures to self-assemble around a filamentous viral particle to form a cylindrical shell, thereby encapsulating the filamentous viral particle.

A further aspect of the present disclosure relates to a method of inhibiting viral infection. This method involves encapsulating a filamentous viral particle with a macromolecular cylindrical shell as described herein, whereby the macromolecular cylindrical shell forms a physical barrier to inhibit filamentous viral particle infection of a cell otherwise susceptible to infection by the filamentous viral particle.

Another aspect of the present disclosure relates to a method of treating an individual for a viral infection. This method involves administering a composition described herein to an individual at a site of viral infection, where the macromolecular cylindrical shell forms a physical barrier that encapsulates filamentous viral particles at the site of viral infection, thereby treating the individual.

The present disclosure relates to trapping entire virus particles within de novo designed macromolecular shells to inhibit molecular interactions between viruses and host cells (see FIG. 1A). Shells are used to augment and work synergistically with a large variety of virus binding moieties, whether by themselves neutralizing or not, to create an effective antiviral agent.

To accomplish this function, shells are made that are large enough to accommodate entire viruses, while also being chemically addressable to allow including virus-specificity conferring moieties on the shell's interior surface. The extended surface of the shells enables functionalization in a multivalent fashion. Multivalency can support tight binding of a target virus even for individually weakly virus-binding molecules, as exemplified in previous experiments with phage nanoparticles engineered to trivalently bind influenza A hemagglutinin (Lauster et al., “Phage Capsid Nanoparticles with Defined Ligand Arrangement Block Influenza Virus Entry,” Nature Nanotechnology 15:373-379 (2020), which is hereby incorporated by reference in its entirety), and with star-shaped DNA aptamer clusters that simultaneously target multiple dengue virus envelope proteins (Kwon et al., “Designer DNA Architecture Offers Precise and Multivalent Spatial Pattern-Recognition for Viral Sensing and Inhibition,” Nature Chemistry 12:26-35 (2020), which is hereby incorporated by reference in its entirety). With shells that fully cover viruses, an even larger degree of multivalency, and thus stronger binding, is envisioned. Modular functionalization of the shells with virus binders will enable using the same type of shell platform to target a variety of viruses. Candidate virus binders could be, e.g., antibodies, designed proteins (Cao et al., “De Novo Design of Picomolar SARS-CoV-2 Miniprotein Inhibitors,” Science 370:426-431 (2020), which is hereby incorporated by reference in its entirety), nucleic acid aptamers, or polymers such as heparan sulphate (Cagno et al., “Sulfate Proteoglycans and Viral Attachment: True Receptors or Adaptation Bias?” Viruses 11 (2019), which is hereby incorporated by reference in its entirety). The shell material, rather than the moieties directly contacting the virus, will mainly prevent access to the viral surface. Therefore, in principle any virus binding molecule could potentially be utilized to convert the shells into an effective virus-neutralizing trap.

The shell concept described herein requires constructing massive molecular complexes that are adaptable to cover the dimensions of viral pathogens (˜ 20 nm to ˜ 500 nm) (see Legendre et al., “Thirty-Thousand-Year-Old Distant Relative of Giant Icosahedral DNA Viruses with a Pandoravirus Morphology,” PNAS 111:4274-4279 (2014), which is hereby incorporated by reference in its entirety), which poses a fundamental nanoengineering challenge.

To build the envisioned virus trap, a programmable icosahedral shell “canvas” was created by adapting symmetry principles known from natural viral capsids. Caspar and Klug elucidated the geometric principles that govern the structure of natural viral capsids in 1962 (Caspar et al., “Physical Principles in the Construction of Regular Viruses,” Cold Spring Harbor Symposia on Quantitative Biology 27:1-24 (1962), which is hereby incorporated by reference in its entirety). According to Caspar and Klug theory, which has been expanded recently (Twarock et al., “Structural Puzzles in Virology Solved with an Overarching Icosahedral Design Principle,” Nature Communications 10:4414 (2019), which is hereby incorporated by reference in its entirety), the number of distinct environments occupied by proteins within an icosahedral capsid is described by its triangulation number (T-number), which can be computed by the arrangement of pentamers and hexamers within an icosahedral capsid (T=h²+hk+k², see FIG. 1B). The total number of proteins required to build a natural capsid is T times sixty. This is because natural protein subunits are, by default, asymmetric and homo-trimerization is minimally required to construct a three-fold symmetric subunit that can assemble into an icosahedral shell with twenty triangular faces. To build larger capsids, viruses use more than one capsid protein or capsid proteins that can adopt different conformations. The structure of natural virus capsids forms the basis for the synthetic programmable icosahedral shell canvasses described herein, which are analogously classified using a T-number.

### BRIEF DESCRIPTION OF THE DRAWINGS

FIGS. 1A-D are schematic illustrations of design principles of the present disclosure. FIG. 1A shows an icosahedral shell encapsulating a virus capsid. FIG. 1B shows a triangular net representation of icosahedral shells. Each triangle represents one of the 20 faces forming an icosahedron. The small triangles represent the triangular building blocks. (h,k) indicates the location of pentamers within a shell. FIG. 1C shows a cylindrical model of DNA-origami triangles assembling into the shells shown in FIG. 1D. The edges of the triangles are beveled and modified with shape-complementary protrusions (light) and recesses (dark). The arrows indicate shape-complementary combinations. FIG. 1D shows icosahedral shells formed by the triangles shown in FIG. 1C. For each shell design, one of its 20 icosahedral faces has been displaced (see FIG. 1B) to help recognize the icosahedral symmetry. α is the bevel angle of the sides, #the number of DNA-origami triangles building the shell.

FIGS. 2A-G relates to structures of shells and of shell subunits. FIG. 2A shows cryo-EM micrographs of assembled shells in free-standing ice (O, T=1) and on lacey carbon grids with carbon support (T=3, T=4). FIGS. 2B-E show cryo-EM reconstructions of shell subunits and fully assembled shells (octahedron to T=4 shells). The two-dimensional class averages show assembled shells from different orientations. FIG. 2F shows EM validation of the T=9 shell design. Top left, cryo-EM reconstructions of the three triangles assembling into a T=9 shell. Top right, negatively stained EM micrograph of assembled shells. Bottom, comparison of slices through a model shell to slices of a tomogram calculated from an EM tilt series. The arrows indicate the positions of pentamers within the shell. FIG. 2G shows cryo-EM reconstruction of a T=1 shell with a central-cavity blocking DNA “spacer” module.

FIGS. 3A-C show shell yield and stability. FIG. 3A are images showing laser-scanned fluorescent images of 0.5% agarose gels showing the assembly of octahedra, T=1, T=3 and T=4 shells at 40° C. with a monomer concentration of 5 nM at different time points. Solid lines give cross-sectional lane intensity profiles from the 1d samples. FIG. 3B show triangle exchange experiments. Cyan: FRET-pair labeled T=1 shells. Orange: unlabeled shells. Symbols give FRET signals measured vs time of incubation in the presence of the indicated concentrations of Mg2+. Errors bars are SEM of duplicate measurements. FIG. 3C are images showing negative-staining TEM image of octahedral shells coated with a 1:1 mixture of oligolysine and oligolysine-PEG and incubated for 1 h and 24 h in 55% mouse serum at 37° C.

FIGS. 4A-K show sculpting on an icosahedral canvas. FIGS. 4A-E show triangular net projection and schematics of different partial shells: half octahedral shell (FIG. 4A), pentamer (FIG. 4B), half T=1 shell (FIG. 4C), ring (FIG. 4D) and T=1 shells lacking a pentagon vertex (FIG. 4E). FIGS. 4F-H show cylindrical models of DNA-origami triangles and corresponding partial shells of the half-octahedral shell (FIG. 4F), half T=1 shell (FIG. 4G) and T=1 shells lacking a pentagon vertex (FIG. 4H). The sides of the triangles are modified with protrusions and recesses. The arrows indicate shape-complementary sides. White crosses indicate deactivated interaction sites. FIGS. 4I-K show cryo-EM 3D reconstructions of the partial shells shown in FIGS. 4F-H. Insets give typical two-dimensional class averages showing assembled shells from different orientations.

FIGS. 5A-G show trapping of hepatitis B virus (HBV) core particles. FIG. 5A shows a negative stain TEM images of HBV core particles trapped in half octahedral shells. Inset: schematic representation of two half octahedral shells (grey) equipped with antibodies (cyan) with a trapped HBV core particle (red). FIG. 5B shows negative stain TEM images of HBV core particles trapped in half T=1 shells. Inset: same as in (FIG. 5A) with a half T=1 shell. FIG. 5C shows a negative stain TEM image of T=1 triangles modified with nine antibodies self-assembled around HBV core particles as templates. Inset: same as in (FIG. 5A) with single triangles. In FIG. 5D: Left, two-dimensional EM class averages; Middle, cryo-EM reconstruction of two octahedral half-shells coordinating a trapped hepatitis-B virus particle. Right: Cut through the octahedral-DNA shell cryo EM map with the HBV core particle trapped. The density around the HBV core particle stems from the antibodies connecting the HBV core particles to the octahedral shell. Red arrows: HBV core particle. Cyan arrows: antibodies connecting the shell to the HBV core particle. FIG. 5E is the same as in FIG. 5D for the half T=1 shell. The electron density thresholds differ, which makes the HBV core particle look thicker in the T=1 half shell compared to the half octahedron (right). FIG. 5F shows negative stain TEM images of T=1 shell with a missing pentagon vertex engulfing up to three HBV core particles. FIG. 5G shows a schematic illustration showing in vitro virus blocking ELISA experiments. Top: Schematic representation of the ELISA experiment. Bottom: All experiments are done at a ratio of antibody (Ab) to HBV of 400:1. The half-shells have 90 antibody binding sites. Solid filled dots indicate 2.5 pM HBV core particles incubated with pre-assembled mixtures of 1 nM oligonucleotide-conjugated capture antibody and various concentrations of half T=1 shells. Inset (i) illustrates low half-shell concentration for which antibodies saturate the half-shell binding sites and excess antibodies are in solution. Inset (ii) illustrates the case when the antibody concentration is equal to the concentration of binding sites on the half-shell; the half-shell is saturated with antibody with little antibody remaining in solution. Inset (iii) illustrates the case where on average 4 antibodies are bound per half-shell. Two controls were performed at the same stochiometric ratio of 400:1 Ab: HBV to quantify virus blocking efficiency as a function of half-shell concentration. The open dot represents a mixture of HBV core particles, antibodies and unfunctionalized T=1 half-shells. The open square represents HBV plus antibody without half-shells. The green dot shows a blocking efficiency of about 80% at only a 5:1 ratio of Ab: half-shells. Error bars are standard deviation of triplicate measurements.

FIGS. 6A-D show neutralization of AAV2 with DNA-origami half shells. FIG. 6A illustrates that successful infection of HEK293T cells with AAV2 results in the expression of eGFP, while cells exposed to AAV2 captured in DNA half shells do not express eGFP. Yellow circles=AAV2, blue Y=anti-AAV2 IgG antibody, grey angled blocks: DNA half shells. FIG. 6B shows TEM images demonstrating capture of AAV2 virus particles within the DNA-origami half shells. Capture was successful in the presence of serum and BSA. Debris from serum can be seen in the TEM images. FIG. 6C is a graph showing quantification of infected cells by flow cytometry for the conditions: AAV2 only, anti-AAV2 applied at IC₅₀ concentration (1 nM), and DNA-origami half shells with anti-AAV2 conjugated to the inside. Anti-AAV2 and DNA half shells were preincubated with AAV2, respectively. The half shells were used at an overall identical antibody concentration as the anti-AAV2 only condition, with ˜ 36 antibodies per shell, and ˜7 half shells per virus particle. Data was quantified using flow cytometry, and is presented as mean±s.d., n=3 biologically independent experiments. One-way ANOVA was performed to test significant inhibition compared the control, both anti-AAV2 alone and half shell origami +anti-AAV2 demonstrated significant neutralization compared to the AAV2 only control (p≤0.0001). Conjugation of anti-AAV2 to DNA-origami half shells results in significantly greater neutralization capacity than free anti-AAV2 (p≤0.001). FIG. 6D shows representative epifluorescent microscopy images demonstrating the expression of eGFP by infected cells. For each of the conditions, eGFP expression (green), cell nuclei (blue) and the overlay are given. Scale bars represent 100 μm.

FIGS. 7A-D illustrate some embodiments of the design principle of triangular subunits. FIG. 7A is a schematic illustration of of T=1 triangle design with a bevel angle alpha. FIG. 7B is a schematic illustration showing a cross-section of a triangle's side containing 4×6 helices in square-lattice packing without (left) and with (right) a bevel angle. The side is turned around the longest helix indicated by ‘0’. d is the distance between the center of two neighboring helices (2.6 nm) and x the radial distance of any helix to helix ‘0’. To transform nm in base pairs, a rise of 0.34 nm per base pair was used. FIGS. 7C-D are schematic illustrations showing the calculation of helix lengths. FIGS. 7C-D, left, show a cylindrical model of a triangle. FIGS. 7C-D, middle, show schematics of the lengths a(x) and b(x) of different helices within the triangle depending on the distance x to helix ‘0’. FIGS. 7C-D, right, provides formulas to calculate the length differences of individual helices. To compensate for geometrical conflicts arising from mismatched backbone positions at the vertices, one single stranded scaffold bases and five single stranded thymine bases for each staple was included at the corners.

FIGS. 8A-D illustrate some embodiments of encapsulation of circular ssDNA and gold nanoparticles in T=1 shell. In FIG. 8A, from left, a schematic shows modified T=1 monomers with ssDNA handles. FIG. 8A, middle, illustrates a circular ssDNA with attached complimentary handles and tagged with CY5. FIG. 8A, right, illustrates encapsulated ssDNA in T=1 shell. Illustration shows half shell, but a complete shell is meant. FIG. 8B, from left, illustrates schematics of an empty shell, encapsulated circular ssDNA, an encapsulated single gold nanoparticle, and encapsulated gold-labeled circular ssDNA. FIG. 8C illustrates slices of negative stain TEM tomograms of each shell in FIG. 8B. FIG. 8D shows laser scanned fluorescent gels of T=1 shells, with and without cargo. Both gel images are taken from the same gel but with different wavelengths. Each column of the gel is color coded with the corresponding particle in sections FIG. 8C and FIG. 8C. The left gel image shows the SYBR safe emission where we see the bands for the scaffold and for assembled shells. On the right gel, emission from CY5 indicates the cargo is in the same position as the assembled shell.

FIGS. 9A-B show triangular net projection and schematics for zig-zag lattice structure (5,0) of tubular, or cylindrical, shells.

FIGS. 10A-B show triangular net projection and schematics for chiral lattice structure (5,3) of tubular, or cylindrical, shells.

FIGS. 11A-B show triangular net projection and schematics for armchair lattice structure (5,5) of tubular, or cylindrical, shells.

FIG. 12 illustrates bevel angles at each of the three vertices of a triangular subunit suitable for forming tubular shells.

FIG. 13 illustrates dihedral mismatch at the vertex of triangular subunit sides of different dihedral angles, as well as proper alignment (matching) at the vertex of sides of the same dihedral angle.

FIG. 14A illustrates a representative cross-section of a T=1 reference triangular subunit's side consisting of 4×6 helices in square-lattice packing with a bevel angle. FIG. 14B illustrates the mismatch alignment of 4×6 helices at the triangular subunit's vertex, and the addition of a ssDNA to allow for alignment of the subunit faces at the vertex.

FIG. 15 is a design diagram illustrating one embodiment of edge wiring to overcome dihedral mismatch.

FIG. 16 is a design diagram illustrating one embodiment without edge wiring.

### DETAILED DESCRIPTION OF THE INVENTION

The present disclosure relates to three-dimensional nucleic acid origami nanostructures that are designed to allow for self-assembly of the nanostructures into a larger structure (e.g., cylindrical, icosahedral, etc.) about the surface of a virus particle, and their use in treatment methods.

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a macromolecular icosahedral shell.

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a macromolecular cylindrical shell.

As referred to herein, DNA (or, more broadly, nucleic acid molecules, including deoxyribonucleotides (DNA), ribonucleotides (RNA), and peptide nucleic acids (PNAs)), used in the molecular structures of the present disclosure refers to a polymeric form of nucleotides of any length. Nucleotides comprise purine and pyrimidine bases, or other natural, chemically or biochemically modified, non-natural, or derivatized nucleotide bases. The backbone of the nucleic acid molecule (also referred to as a polynucleotide (comprising nucleotides)), can comprise sugars and phosphate groups, as may typically be found in DNA or RNA, or modified or substituted sugar or phosphate groups. A polynucleotide may comprise modified nucleotides, such as methylated nucleotides and nucleotide analogs.

Typically, a nucleic acid molecule will comprise phosphodiester bonds. However, nucleic acid molecules may comprise a modified backbone comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. As will be appreciated by a person of skill in the art, all of these nucleic acid analogs may find use as helper strands or as part of a polynucleotide used to generate the nanostructures described herein. In addition, mixtures of naturally occurring nucleic acids and analogs can be made and are also suitable in the nanostructures described herein. PNAs include peptide nucleic acid analogs, which may have increased stability.

Thus, nucleic acid of various forms and conformations may be used for generating the three-dimensional nucleotide molecular structures described herein, including right-handed DNA, right-handed RNA, PNA, locked nucleic acid (LNA), threose nucleic acid (TNA), glycol nucleic acid (GNA), bridged nucleic acid (BNA), phosphorodiamidate morpholino oligo (PMO), as well as nucleotide analogues, such as non-Watson-Crick nucleotides dX, dK, ddX, ddK, dP, dZ, ddP, and ddZ.

In some embodiments, a three-dimensional molecular structure of the present disclosure comprises one or more distinct polymeric nucleic acid structures (e.g., at least 20, at least 50, at least 100, or at least 1000 or more distinct nucleic acid molecules). The nucleic acids may be single stranded or double stranded, or contain portions of both double stranded or single stranded sequence. The nucleic acid may be DNA, either or both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, and the like. Such nucleic acids comprise nucleotides and nucleoside and nucleotide analogs, and modified nucleosides such as amino modified nucleosides.

In some embodiments of the present disclosure, the nucleic acid nanostructure is DNA origami. DNA origami is a method of generating DNA artificially folded at nanoscale, creating an arbitrary three dimensional shape that may be used as a scaffold for trapping inside, or capturing, an entity. Methods of producing DNA nanostructures of the origami type have been described, for example, in U.S. Pat. No. 7,842,793, which is hereby incorporated by reference in its entirety. DNA origami involves the folding of a long single strand of viral DNA (for example) aided by multiple smaller “staple” strands. These shorter strands bind the longer strand in various places, resulting in the formation of a 3D structure. The three-dimensional nucleotide molecular structures of the present disclosure may use numerous shorter single strands of nucleic acids (helper strands) (e.g., DNA) to direct the folding of a longer, single strand of polynucleotide (which is called, in DNA nanostructure nomenclature, the scaffold strand) into desired shapes, such as a nanoscale triangular subunit, that are usually between 100-5000 nm in diameter. A plurality of nanoscale triangular subunits have a configuration that allows those nanoscale triangular subunits to self-assemble in the form of a macromolecular icosahedral or cylindrical shell. The icosahedral or cylindrical shell may be on the order of about 100 nm to 5000 nm, but larger scaffolds of 10, 15, or 20 μm may also be achieved and used, depending on the context.

Nucleic acid nanotechnology makes use of the fact that, due to the specificity of Watson-Crick base pairing, only portions of the strands which are complementary to each other will bind to each other to form duplex. Construction of nucleic acid nanostructures has been described in several publications, including PCT Publication No. WO 2008/039254; U.S. Patent Application Publication No. 2010/0216978; PCT Publication No. WO 2010/0148085; U.S. Pat. Nos. 5,468,851; 7,842,793; Dietz et al., “Folding DNA Into Twisted and Curved Nanoscale Shapes,” Science 325:725-730 (2009); and Douglas et al., “Self-Assembly of DNA Into Nanoscale Three-Dimensional Shapes,” Nature 459:414 (2009); which are hereby incorporated by reference in their entirety, amongst others.

Natural or artificial sequences of DNA can be programmed to generate a three-dimensional (3D) structure. Usually, DNA-based nanostructures make use of a single strand of DNA which is induced into a 3D conformation by the binding of complementary, shorter DNA strands. In contrast, RNA folds into 3D by forming tertiary RNA motifs, based on RNA-RNA interactions within the same molecule Nanostructures based on folded single-stranded DNA are also feasible. RNA duplexes are an alternative for generating RNA 3D structures.

In some embodiments, the three-dimensional nucleotide molecular structure of the present disclosure is a structure of joined tiles of DNA origami, in the form of the nanoscale triangular subunits, which self-assemble to form the icosahedral or cylindrical structure. Inducible nucleic acid nanostructures have been described, for example, by Andersen et al., “Self-Assembly of a Nanoscale DNA Box with a Controllable Lid,” Nature 459:73-77 (2009); Dietz et al., “Folding DNA Into Twisted and Curved Nanoscale Shapes,” Science 325:725-730 (2009); Voigt et al., “Single-Molecule Chemical Reactions on DNA Origami,” Nat. Nanotechnology 5:200-203 (2010); and Han et al., “DNA Origami with Complex Curvatures in Three-Dimensional Space.” Science 332:342-346 (2011); which are hereby incorporated by reference in their entirety). A software package for designing nucleic acid nanostructures is available at www.cdna.dk/origami.

As discussed in more detail below, three-dimensional nucleotide molecular structures described herein self-assemble to form a macromolecular shell. In some embodiments, the three-dimensional nucleotide molecular structure is a nanoscale triangular subunit. In some embodiments, all triangle bevel angles for a particular target shell are the same, however this need not always be the case.

In designing the three-dimensional nucleotide molecular structures and macromolecular shells described herein, iterative design may be used with, e.g., caDNAno (see Douglas et al., “Rapid Prototyping of 3D DNA-Origami Shapes with caDNAno,” Nucleic Acids Research 37:5001-5006 (2009), which is hereby incorporated by reference in its entirety) paired with elastic-network-guided molecular dynamics simulations (Maffeo et al., “De Novo Reconstruction of DNA Origami Structures Through Atomistic Molecular Dynamics Simulation,” Nucleic Acids Research 44:3013-3019 (2016), wich is hereby incorporated by reference in its entirety) to produce candidate designs.

Approximate target bevel angles for helical connectivity of triangle edges may be tuned in the vertices, and candidate designs may be encoded in DNA sequences using known methods of DNA origami (see Douglas et al., “Self-Assembly of DNA into Nanoscale Three-Dimensional Shapes,” Nature 459:414-418 (2009); Rothemund, “Folding DNA to Create Nanoscale Shapes and Patterns,” Nature 440:297-302 (2006); which are hereby incorporated by reference in their entirety) and self-assembled in one-pot reaction mixtures (see Wagenbauer et al., “How we Make DNA Origami,” Chembiochem: A European Journal of Chemical Biology (2017), which is hereby incorporated by reference in its entirety).

In some embodiments of the three-dimensional nucleotide molecular structure, the plurality of nanoscale triangular subunits self-assemble by lateral edge-to-edge stacking via base-pair stacking, as described in more detail in the Examples below.

In some embodiments, each of the three edges of the nanoscale triangular subunits mate with only one of the other two edges, as described in more detail in the Examples below.

In some embodiments, the three sides of the nanoscale triangular subunit comprises bevel angles of about 10.4°, about 10.4°, and about −5.3°, although other angles could be used depending on the desirable overall design structure and target.

In some embodiments, target bevel angles in a triangle subunit must be matched within a range of ±5°, although other variations may also be used, such as ±4°, ±3°, ±2°, ±1°, or even ±0.5°, ±0.4°, ±0.3°, ±0.2°, or ±0.1°.

In some embodiments, one side of the nanoscale triangular subunit has a different bevel angle from the other two sides, which causes misalignment at an associated vertex, and the three-dimensional nucleotide molecular structure further comprises an additional ss-DNA molecule self-assembled into the nanoscale triangular subunit along the one side, as discussed in more detail in the Examples below.

In some embodiments, the additional ss-DNA molecule is positioned along a base surface of the nanoscale triangular subunit, as discussed in more detail in the Examples below.

In some embodiments, the three-dimensional nucleotide molecular structure is directed to coat a virus shell by targeting an inner surface (or base surface) of the nanostructure to the external surface of the virus particle. That can be achieved by tethering or linking a targeting moiety to the nanoscale triangular subunit along a base surface.

The targeting moiety can be a virus-specific receptor, antibody, active antibody fragment, nucleic acid aptamer, or peptide antibody mimic. These exemplary targeting moieties can be tethered to the base surface using a ss-DNA molecule covalently linked to the targeting moiety such that the targeting moiety has its active surface exposed on the base surface of the nanoscale triangular subunit.

As referred to herein, an “aptamer” is a relatively short nucleic acid (DNA, RNA, or a combination of both) sequence that binds with high avidity to a variety of proteins. Aptamers are generally about 25-40 nucleotides in length and have molecular weights in the range of about 18-25 kDa. Aptamers with high specificity and affinity for targets can be obtained by an in vitro evolutionary process termed SELEX (systemic evolution of ligands by exponential enrichment) (see, e.g., Zhang et al., Arch. Immunol. Ther. Exp. 52:307-315 (2004), which is hereby incorporated by reference in its entirety).

As referred to herein, “antibodies” relate to naturally derived, or naturally produced antibodies, which may be polyclonal or monoclonal. Alternatively, the antibodies may be synthetically produced by e.g., chemical synthesis, or recombinantly produced through the isolation of the specific mRNA from the respective antibody-producing cell or cell line. The specific mRNA shall then undergo standard molecular biology manipulations (obtaining cDNA, introducing the cDNA into expression vectors, etc.) in order to generate a recombinantly produced antibody.

The generation of polyclonal antibodies against proteins is a technique well known in the art, as described, e.g., in Chapter 2 of Current Protocols in Immunology, John E. Coligan et al. (eds.), Wiley and Sons Inc., which is hereby incorporated by reference in its entirety.

The technique of generating monoclonal antibodies is described in many articles and textbooks, such as the above-noted Chapter 2 of Current Protocols in Immunology, Kohler and Milstein (Kohler and Milstein (1975) Nature 256:495-497), and in U.S. Pat. No. 4,376,110, which are hereby incorporated by reference in their entirety.

“Antibody” also includes both intact molecules as well as fragments thereof, such as, for example, scFv, Fv, Fab′, Fab, diabody, linear antibody, F(ab′)2 antigen binding fragment of an antibody which are capable of binding antigen (Wahl et al., “Improved Radioimaging and Tumor Localization with Monoclonal F(ab′)2,” J. Nucl. Med. 24:316-325 (1983), which is hereby incorporated by reference in its entirety.

In some embodiments, the three-dimensional DNA molecular structure comprises a targeting moiety that binds to a viral capsid protein. A “capsid protein” is a protein monomer. Capsid proteins can assemble together to form a capsomere (e.g., a pentamer of capsid proteins). A “capsomere” is a subunit of a viral capsid, which is an outer covering of protein that protects the genetic material of a virus such as, for example, human papillomavirus (HPV).

Capsids are broadly classified according to their structure. The majority of the viruses have capsids with either helical or icosahedral structure. The icosahedral shape, which has 20 equilateral triangular faces, approximates a sphere, while the helical shape resembles the shape of a spring, taking the space of a cylinder but not being a cylinder itself. The capsid faces may include one or more proteins.

Some viruses are enveloped, meaning that the capsid is coated with a lipid membrane known as the viral envelope. The envelope is acquired by the capsid from an intracellular membrane in the virus' host.

Once a virus has infected a cell and begins replicating itself, new capsid subunits are synthesized using the protein biosynthesis mechanism of the cell. In some viruses, including those with helical capsids and especially those with RNA genomes, the capsid proteins co-assemble with their genomes. In other viruses, especially more complex viruses with double-stranded DNA genomes, the capsid proteins assemble into empty precursor procapsids that include a specialized portal structure at one vertex. Through this portal, viral DNA is translocated into the capsid.

An external capsid protein is a capsid protein that is exposed on the surface of a VLP. A virus-like particle, or VLP, refers to an organized capsid-like structure (e.g., roughly spherical or cylindrical in shape) that comprises self-assembling ordered arrays capsomeres and does not include a viral genome. In some embodiments, the virus-like particles are morphologically and antigenically similar to authentic virions, but they lack viral genetic material (e.g., viral nucleic acid), rendering the particles non-infectious.

In some embodiments, the targeting moiety is tethered to a ss-DNA molecule that hybridizes to a discrete location along the base surface.

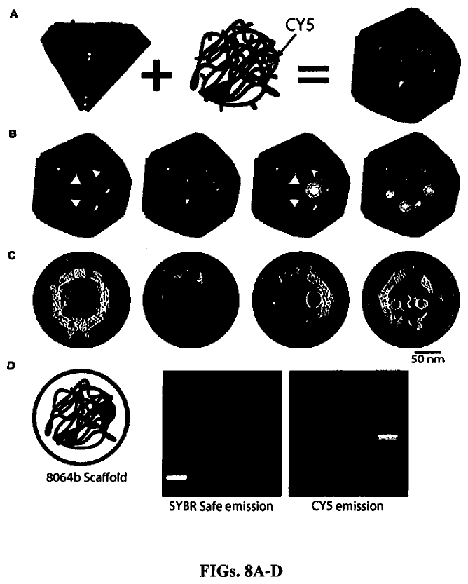

Design and location of targeting moieties to three-dimensional nucleotide structures described herein are described in more detail in with reference to FIGS. 8A-D.

Another aspect of the present disclosure relates to a macromolecular cylindrical or icosahedral shell formed by self-assembly of a plurality of the three-dimensional DNA molecular structures described herein.

All the various embodiments described above for the three-dimensional nucleotide molecular structures may also be applied to this aspect of the present disclosure.

In some embodiments of the macromolecular cylindrical shell, the three-dimensional DNA molecular structures are self-assembled by lateral edge-to-edge stacking via base-pair stacking, and the macromolecular cylindrical shell further comprises a linking agent that binds to two edge-to-edge stacked nanoscale triangular subunits, as described above and in the Examples below.

In some embodiments, cylindrical shapes are formed. In some embodiments, icosahedral or spherical shapes are formed.

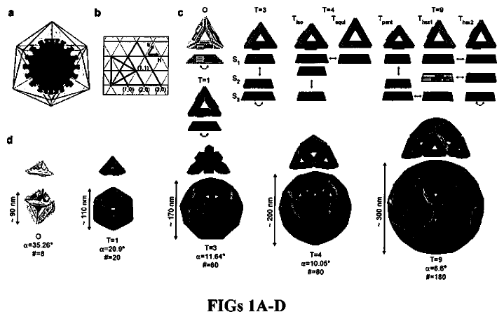

In some embodiments of forming a spherical or icosahedral shape, pseudo-symmetric triangular subunits (see FIG. 1C) may be designed based on multi-layer DNA origami concepts previously described (see e.g., Douglas et al., “Self-Assembly of DNA into Nanoscale Three-Dimensional Shapes,” Nature 459:414-418 (2009); Castro et al., “A Primer to Scaffolded DNA Origami,” Nature Methods 8:221-229 (2011); which are hereby incorporated by reference in their entirety), as discussed above and in the Examples below.

In some embodiments, each side of a triangular subunit is the equivalent of one protein subunit of a natural viral capsid. Overall scale and type may be controlled by geometric instructions provided by the triangular subunits. These instructions are given by the choice of the length, the topological binding pattern (see Gerling et al., “Dynamic DNA Devices and Assemblies Formed by Shape-Complementary, Non-Base Pairing 3D Components,” Science 347:1446-1452 (2015), which is hereby incorporated by reference in its entirety), and the bevel angle of each triangular edge. In some embodiments, each triangular edge may represent, e.g., one protein, in which case the Caspar and Klug triangulation number gives the number of unique triangular edges required to build a particular icosahedral canvas shell. Hence, as described in more detail in the Examples below, T=1 and T=3 shells may both be built with a single triangle, with three identical edges for T=1 and three different edges for a T=3 shell (see FIGS. 1C-D, left). A T=4 shell requires two separate triangular subunits, for example, one triangle with three unique edges and another with three identical edges (see FIGS. 1C-D, middle). A T=9 shell requires three different triangles, each having three unique edges (see FIGS. 1C-D, right). The greater the T number, the greater the overall number of triangles per target shell, given by 20 T.

In some embodiments, the macromolecular cylindrical shell has a 5,0 lattice structure, a 5,3 lattice structure, or a 5,5 lattice structure.

To stabilize macromolecular shells described herein for certain applications (e.g., in physiological fluids), the macromolecular shells may be assembled and then UV point welded by techniques previously described (see Gerling et al., “Sequence-Programmable Covalent Bonding of Designed DNA Assemblies,” Sci. Adv. 4: caau 1157 (2018), which is hereby incorporated by reference in its entirety). Such techniques may be applied to create additional internal covalent bonds across the stacking contacts in the triangle subunits.

In some embodiments, macromolecular shells are coated (e.g., but without limitation, with a mixture of oligolysine and PEG oligolysine, as has been described, for example, in Ponnuswamy et al., “Oligolysine-Based Coating Protects DNA Nanostructures from Low-Salt Denaturation and Nuclease Degradation,” Nature Comm. 8:15654 (2017), which is hereby incorporated by reference in its entirety.

In some embodiments, the macromolecular shell is configured to encapsulate a filamentous virus particle, although encapsulation of other virus particles having different shapes (e.g., icosahedral) is also contemplated. A number of filamentous viruses are known to include many plant viruses as well as a number of animal viruses, including normally icosahedral animal viruses that nevertheless generate filamentous virus particles. Examples of normally filamentous virus particles include, without limitation, all Filoviridae such as Cuevavirus (e.g., Lloviu virus), Dianlovirus (e.g., Mănglà virus), Ebolavirus (e.g., Bombali virus, Bundibugyo virus, Reston virus, Sudan virus, Taï Forest virus, and Ebola virus), and Marburgvirus (e.g., Marburg virus, and Ravn virus). Others include Nipah and Hendra viruses. Examples of normally icosahedral viruses that generate filamentous forms include, without limitation, Influenza A and B viruses, Measles virus, Respiratory Syncytial virus, and African swine fever virus.

A further aspect of the present disclosure relates to a composition comprising a plurality of three-dimensional DNA molecular structures described herein in a carrier.

Another aspect of the present disclosure relates to a composition comprising a plurality of macromolecular cylindrical shells as described herein in a carrier.

A further aspect of the present disclosure relates to a composition comprising a plurality of three-dimensional DNA molecular structures described herein and a plurality of macromolecular cylindrical shells as described herein in a carrier.

In some embodiments of the compositions of the present disclosure, the carrier is an aqueous carrier.

In some embodiments of the compositions of the present disclosure, the carrier is a pharmaceutically acceptable carrier.

In some embodiments, the pharmaceutically acceptable carrier is suitable for oral, mucosal, topical, or systemic delivery to a subject, such as a mammalian subject, including a human.

In some embodiments, the pharmaceutically acceptable carrier is suitable for delivery intranasally or by inhalation.

Three-dimensional DNA molecular structures described herein and/or macromolecular cylindrical shells described herein, and their compositions as described herein, can be used to encapsulate viral particles, including filamentous viral particles; inhibit viral infections; and treat individuals.

In other words, the present disclosure also relates to compositions containing the DNA origami nanostructures and the use of such compositions to cause filamentous viral particle encapsulation or to treat an individual for a viral infection by a filamentous virus. Such treatment can be prospective (i.e., to inhibit infection following exposure) or therapeutic (i.e., to treat an existing infection to minimize damage and shorten the infection and illness accompanying the same).

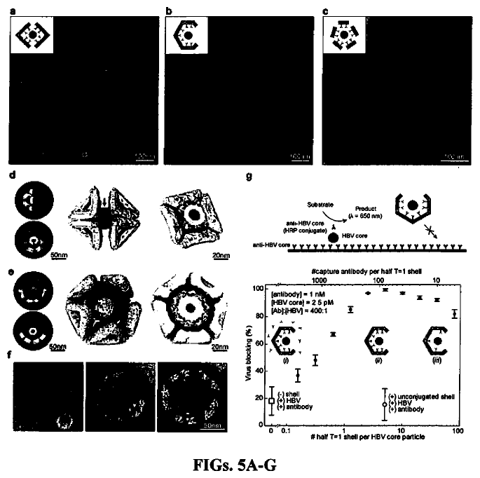

In the present disclosure, viruses can be trapped in, or coordinated by, pre-assembled shell segments (e.g., icosahedral shell segments) featuring sufficiently large apertures (see FIGS. 5A-B). Alternatively, protective shells can be formed directly on the surface of virus particles (see FIG. 5C).

Such treatment is effected by administering nucleotide structures and shells described herein capable of encapsulating the filamentous virus particles to the subject. As used herein, the term “subject” refers to an animal, preferably a mammal such as a human.

Thus, a further aspect of the present disclosure relates to a method of encapsulating a filamentous viral particle. This method involves providing a plurality of the three-dimensional DNA molecular structures described herein, and allowing said three-dimensional DNA molecular structures to self-assemble around a filamentous viral particle to form a cylindrical shell, thereby encapsulating the filamentous viral particle.

Another aspect of the present disclosure relates to a method of inhibiting viral infection. This method involves encapsulating a filamentous viral particle with a macromolecular cylindrical shell as described herein, whereby the macromolecular cylindrical shell forms a physical barrier to inhibit filamentous viral particle infection of a cell otherwise susceptible to infection by the filamentous viral particle.

And yet a further aspect of the present disclosure relates to a method of treating an individual for a viral infection. This method involves administering a composition as described herein to an individual at a site of viral infection, where the macromolecular cylindrical shell forms a physical barrier that encapsulates filamentous viral particles at the site of viral infection, thereby treating the individual.

In some embodiments, the three-dimensional nucleotide molecular structures and/or macromolecular shells described herein can be administered to the subject per se, or in a pharmaceutical composition where it is mixed with suitable carriers or excipients.

As used herein, a “pharmaceutical composition” refers to a preparation of one or more of the active ingredients described herein (i.e., a three-dimensional nucleotide molecular structure and/or macromolecular shell) with other chemical components such as physiologically suitable carriers and excipients. The purpose of a pharmaceutical composition is to facilitate administration of a compound to an organism.

As used herein, the term “active ingredient” refers to the individual three-dimensional nucleotide molecular structure and/or macromolecular shell formed by self-assembly of a plurality of the three-dimensional nucleotide molecular structures as described herein, as well as partial and complete assemblies thereof which are accountable for the intended biological effect.

In alternative embodiments, the individual three-dimensional nucleotide molecular structure and/or macromolecular shell formed by self-assembly of a plurality of the three-dimensional nucleotide molecular structures are used as a vehicle for delivering a pharmaceutical agent. Encapsulation and delivery of any known or later development pharmaceutical agent is contemplated.

The phrases “physiologically acceptable carrier” and “pharmaceutically acceptable carrier,” which may be used interchangeably, refer to a carrier or a diluent that does not cause significant irritation to an organism upon administration and does not abrogate the biological activity and properties of the administered active ingredient. An adjuvant is included under these phrases.

The term “excipient” used herein refers to an inert substance added to a pharmaceutical composition to further facilitate administration of an active ingredient. Examples of excipients include, without limitation, calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, PA, which is hereby incorporated by reference in its entirety.

Suitable routes of administration include, for example, oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections. In some embodiments, routes of administration may include, without limitation, intranasal delivery and inhalation.

The pharmaceutical compositions described herein may be administered in a local rather than systemic manner, for example, via injection of the pharmaceutical composition directly into a tissue region of a patient.

Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in the present disclosure may be formulated in a conventional manner using one or more physiologically acceptable carriers comprising excipients and auxiliaries, which facilitate processing of the active ingredients into preparations that can be used pharmaceutically. Proper formulation may be dependent on the route of administration chosen.

For injection, the active ingredients of the pharmaceutical composition may be formulated in aqueous solutions, such as in physiologically compatible buffers such as Hank's solution, Ringer's solution, or physiological salt buffer. For transmucosal administration, penetrants appropriate to the barrier to be permeated may be used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents, such as cross-linked polyvinyl pyrrolidone, agar, or alginic acid, or a salt thereof, such as sodium alginate, may be added.

Dragee cores are provided with suitable coatings. For this purpose, concentrated sugar solutions may be used that may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present disclosure are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical compositions described herein may be formulated for parenteral administration, e.g., by bolus injection or continuous infusion. Formulations for injection may be presented in unit dosage form, e.g., in ampules or in multi-dose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared, as appropriate, with oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

In some embodiments, the active ingredient may be in powder form for constitution with a suitable vehicle, e.g., a sterile, pyrogen-free, water-based solution, before use.

Sustained-release (SR), extended-release (ER, XR, or XL), time-release or timed-release, controlled-release (CR), or continuous-release (CR or Contin) pills are tablets or capsules formulated to dissolve slowly and release a drug over time. Sustained-release tablets are formulated so that the active ingredient is embedded in a matrix of insoluble substance (e.g., acrylics, polysaccharides, etc.) such that the dissolving drug diffuses out through the holes in the matrix. In some SR formulations the matrix physically swells up to form a gel, so that the drug has first to dissolve in matrix, then exit through the outer surface.

The difference between controlled release and sustained release is that controlled release is perfectly zero order release. That is, the drug releases with time irrespective of concentration. On the other hand, sustained release implies slow release of the drug over a time period. It may or may not be controlled release.

Pharmaceutical compositions suitable for use in the context of the present disclosure include compositions where the active ingredients are contained in an amount effective to achieve the intended purpose. More specifically, a “therapeutically effective amount” means an amount of active ingredient(s) effective to prevent, alleviate, or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the present disclosure, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures, or experimental animals. The data obtained from in vitro, cell culture assays, and animal studies can be used in formulating a range of dosage for use in human. The dosage may vary depending upon the dosage form employed and the route of administration utilized. The exact formulation, route of administration, and dosage can be chosen by the individual physician in view of the patient's condition (see, e.g., Fingl et al., “The Pharmacological Basis of Therapeutics,” Ch. 1, p. 1 (1975), which is hereby incorporated by reference in its entirety).

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present disclosure may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the present disclosure formulated in a pharmaceutically acceptable carrier may also be prepared, placed in an appropriate container, and labeled for treatment of an indicated condition, as further detailed above.

### EXAMPLES

The examples below are intended to exemplify the practice of embodiments of the disclosure but are by no means intended to limit the scope thereof.

### Example 1—Design and Testing of Programmable Icosahedral Shell System for Virus Trapping

### Shell Canvas Design Principles

To implement the icosahedral canvas concept, pseudo-symmetric triangular subunits (FIG. 1C) were designed based on multi-layer DNA origami concepts (Douglas et al., “Self-Assembly of DNA into Nanoscale Three-Dimensional Shapes,” Nature 459:414-418 (2009); Castro et al., “A Primer to Scaffolded DNA Origami,” Nature Methods 8:221-229 (2011); which are hereby incorporated by reference in their entirety). Each side of a triangular subunit is the equivalent of one protein subunit of a natural viral capsid. The overall canvas scale and type are controlled by geometric instructions provided by the triangular subunits. These instructions are given by the choice of the length, the topological binding pattern (Gerling et al., “Dynamic DNA Devices and Assemblies Formed by Shape-Complementary, Non-Base Pairing 3D Components,” Science 347:1446-1452 (2015), which is hereby incorporated by reference in its entirety), and the bevel angle of each triangular edge. Since in this system each triangular edge represents one protein, the Caspar and Klug triangulation number gives the number of unique triangular edges required to build a particular icosahedral canvas shell. Hence, T=1 and T=3 shells may both be built with a single triangle, with three identical edges for T=1 and three different edges for a T=3 shell (FIGS. 1C-D, left). A T=4 shell requires two separate triangular subunits, for example, one triangle with three unique edges and another with three identical edges (FIGS. 1C-D, middle). A T=9 shell requires three different triangles, each having three unique edges (FIGS. 1C-D, right). The greater the T number, the greater the overall number of triangles per target shell, given by 20 T. Design solutions were used in which all triangle bevel angles for a particular target shell were the same. While T=9 was the largest canvas set out to be built, triangular subunits were also designed for a smaller octahedral container (“O”) (FIGS. 1C-D, left).

### Subunit and Shell Canvas Assembly

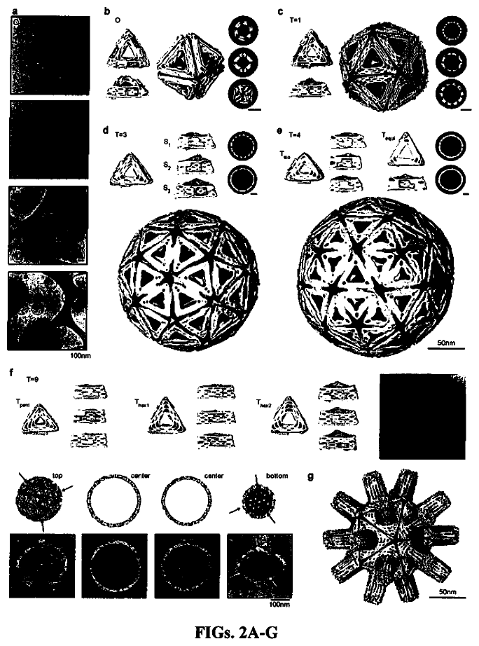

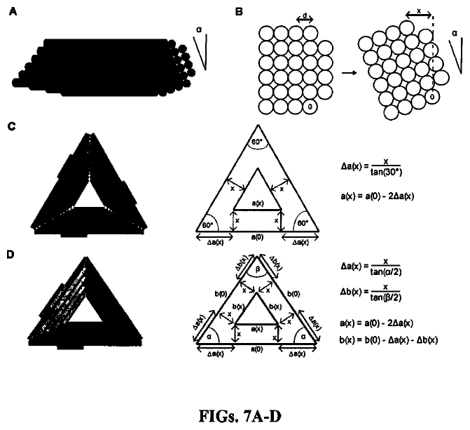

Iterative design was used with caDNAno (Douglas et al., “Rapid Prototyping of 3D DNA-Origami Shapes with caDNAno,” Nucleic Acids Research 37:5001-5006 (2009), which is hereby incorporated by reference in its entirety) paired with elastic-network-guided molecular dynamics simulations (Maffeo et al., “De Novo Reconstruction of DNA Origami Structures Through Atomistic Molecular Dynamics Simulation,” Nucleic Acids Research 44:3013-3019 (2016), wich is hereby incorporated by reference in its entirety) to produce candidate designs. To approximate target bevel angles, the helical connectivity of the triangle edges were tuned in the vertices (FIG. 7). These candidate designs were encoded in DNA sequences using the methods of DNA origami (Douglas et al., “Self-Assembly of DNA into Nanoscale Three-Dimensional Shapes,” Nature 459:414-418 (2009); Rothemund, “Folding DNA to Create Nanoscale Shapes and Patterns,” Nature 440:297-302 (2006); which are hereby incorporated by reference in their entirety) and self-assembled in one-pot reaction mixtures (Wagenbauer et al., “How we Make DNA Origami,” Chembiochem: A European Journal of Chemical Biology (2017), which is hereby incorporated by reference in its entirety). Gel-electrophoretic folding quality analysis demanded some design iterations to improve triangular subunit assembly yields. To validate the 3D structures of the designed triangles, all triangle subunits were studied using cryo transmission electron microscopy (cryo-EM) single particle analysis (FIGS. 2A-G). The resulting 3D electron maps had resolutions ranging from 13 to 22 Angstroms, which allowed evaluation of the overall 3D shapes, the observed versus desired bevel angles, the correct formation of the binding patterns, and the occurrence of systematic folding defects. For instance, one triangle variant (Tₕₑₓ₁) had a defective vertex, which decreased its ability to form lateral edge-to-edge interactions. Based on the cryo-EM data, the design was refined and the defect eliminated.

The triangle variants self-assembled successfully into the designed icosahedral shells, as confirmed by direct imaging with cryo-EM (FIG. 2A). Inspection of individual particles (FIG. 2A) and of 2D class averages (FIGS. 2B-F) revealed particles displaying the designed symmetries. For example, the three symmetry axes of the octahedron (4-fold, 3-fold, 2-fold, FIG. 2B) and T=1 shell (5-fold, 3-fold, 2-fold, FIG. 2C) can be clearly seen. For the higher-T-number shells, the underlying triangular net predicted from the Caspar-and-Klug representation became clearly visible (FIGS. 2D-F). 3D EM maps were determined from the image data by imposing the respective symmetry (FIG. 2B-E). The resulting maps had resolutions ranging from 20 to 40 Angstrom. For the octahedron and T=1 shell, 3D maps reconstructed without imposing any a priori symmetry superimposed well with the sibling reconstructed with imposed symmetry. Cryo EM maps of shells that lacked one or multiple triangles were classified and treated separately from complete shells to assess quality and yield. The largest T=9 shells were imaged using negative stain EM tomography (FIG. 2F). Sections through tomograms of assembled T=9 shells show fully closed shells as well as the correct arrangement of pentamers according to the designed T-number (arrows in FIG. 2F).

To elucidate effects of orientational specificity of subunit-subunit interactions, the bevel angle of the T=1 subunits were varied from the ideal geometry (α=20.9°). Two additional variants of the T=1 triangle whose bevel angles deviated by +5° or −5° from the icosahedral ideal were designed. The decrease or increase of the bevel angle caused the appearance of larger or smaller, often defective, assemblies in addition to T=1 shells, respectively. Based on these data it was concluded that the correct target bevel angle in a T=1 triangle subunit must be matched within a range of +−5°.

As a demonstration for a route for sealing the remaining cavities in the shells, a DNA brick having a triangular cross-section was built roughly corresponding to the dimensions of the triangular cavity in the shell subunits. The brick was anchored via multiple attachment points to the outer surface of a T=1 shell triangle. A structure of the spiky T=1 shell was solved using cryo-EM single particle analysis (FIG. 2G). The resulting map overlaps well with those of the unmodified T=1 shell, but the central cavity of the triangle subunits is now blocked by the added brick module. The fact that the cavity-plugging with the DNA brick worked indicates robustness and structural modularity of the shells. The brick may also be considered as a mimic for previously described DNA-based membrane channels (Langecker et al., “Synthetic Lipid Membrane Channels Formed by Designed DNA Nanostructures,” Science 338:932-936 (2012), which is hereby incorporated by reference in its entirety) or for any other functional module that one wishes to attach to a shell.

### Shell Yield and Stability

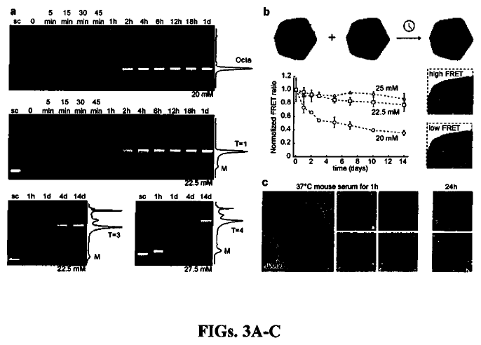

Practical aspects such as assembly yield and stability were evaluated in physiological conditions where the system is ultimately expected to be applied. Low-density gel electrophoretic mobility analysis (FIG. 3A) revealed that shell assembly proceeded by disappearance of the triangular monomers, appearance of a smear indicating the presence of oligomeric species, followed by emergence of a dominant high intensity band, corresponding to the fully formed shells. Octahedra and T=1 shells formed within 15 and 60 minutes, respectively, which is sufficiently fast to enable self-assembly of these shells directly during the one-pot triangle-folding reaction. They formed with a final complete shell yield of ˜95% and ˜70%, respectively. The T=3 and T=4 shells formed with about 40% yield (FIG. 3A). Subunit-exchange experiments with fluorescently labeled subunits revealed that under shell-favoring conditions triangles that are incorporated in closed shells do not exchange with solution (FIG. 3B). Under equilibrium conditions, triangles do exchange (FIG. 3B). To stabilize the shells for application in physiological fluids, the shells were first assembled and then UV point welding (see Gerling et al., “Sequence-Programmable Covalent Bonding of Designed DNA Assemblies,” Sci. Adv. 4: eaau1157 (2018), which is hereby incorporated by reference in its entirety) applied to create additional internal covalent bonds across the stacking contacts in the triangle subunits. The shells were then coated with a mixture of oligolysine and PEG oligolysine (Ponnuswamy et al., “Oligolysine-Based Coating Protects DNA Nanostructures from Low-Salt Denaturation and Nuclease Degradation,” Nature Comm. 8:15654 (2017), which is hereby incorporated by reference in its entirety). This two-step treatment allowed the successful transfer of the shells into mouse serum, where the shells remained intact for up to 24 h (FIG. 3C).

### Sculpting on the Icosahedral Canvas

By changing the geometry of the shape-complementary topographic features, the triangular subunits can be programmed to cover only user-defined areas on the icosahedral canvas. To create full shells, only the minimum number of different topographic interaction patterns (“symmetries”) is implemented as discussed herein. Introducing additional types of topographic edge-to-edge interactions per triangular subunit allows reducing the symmetry in which the subunit may be integrated in the canvas. Furthermore, the stacking interactions can be modularly activated and de-activated, for example by shortening a strand terminus involved in a stacking contact or by adding unpaired thymidine terminal strand extensions. Together, these features enable sculpting a variety of objects on the icosahedral canvas in a programmable fashion, including full shells, pentagonal vertices, (spherical) half-shells, and shells with virus-sized openings using rational design decisions.

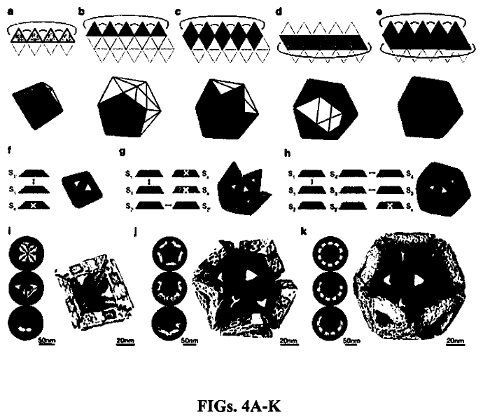

To design such objects, the triangular net projection of the chosen icosahedral canvas type was used as a drawing board (FIGS. 4A-E). For example, in order to prepare half instead of full octahedra, complementary lock-and-key interactions of two edges of the triangular subunit are needed and one edge interaction must be deactivated (FIG. 4A). A pentagonal dome can be analogously created based on the T=1 icosahedral canvas (FIG. 4B). Building an icosahedral half shell requires two different triangular subunits, one that forms the pentagonal dome, and another that specifically docks onto the edges of the pentamer (FIG. 4C). A ring-like “sheath” may also be built by two triangles (FIG. 4D). To build a T=1 shell variant with one missing pentagon vertex, three triangular subunit variants with a specific interaction pattern are needed (FIG. 4E). The above discussed design variants were practically implemented using appropriately modified triangular building blocks (FIGS. 4F-H). The building blocks self-assembled successfully into the desired higher-order objects based on their icosahedral canvas, which was validated experimentally by determining cryo EM solution structures (FIGS. 4I-K) and negative stain TEM images.

### Virus Trapping

Viruses can be trapped in, or coordinated by, pre-assembled icosahedral shell segments featuring sufficiently large apertures (FIGS. 5A-B). Alternatively, protective shells can be formed directly on the surface of virus particles (FIG. 5C). Both approaches are illustrated in experiments performed with hepatitis B virus core particles (HBV) (FIGS. 5A-C inset, red). To confer specificity to HBV, anti-HBc 17H7 (Isotype IgG-2b) were conjugated to the DNA shells by hybridization of ssDNA-labeled antibodies to a set of anchor points on the triangle subunits (FIGS. 5A-C inset, cyan). No HBV binding was observed in the absence of HBV antibodies, nor in the presence of antibodies specific for other targets.

3D cryo EM maps of octahedral and T=1 half shells with trapped HBV core particles were determined (FIGS. 5D-E). For the half-octahedral variant, the majority of particles were composed of two opposing half octahedra coordinating a single HBV core particle in their middle (FIG. 5D). The micrographs and the cryo EM map also reveal signatures reflecting the antibodies that link the DNA shell to the trapped HBV core particle (FIG. 5D, right). Similar antibody signatures may be found in the image data with the half T=1 shell-HBV complex (FIG. 5E, right). HBV core particles were also trapped in larger T=1 shell variants with a missing pentagon vertex (FIG. 5F), which can accommodate multiple HBV particles in their interior cavities (FIG. 5F).

To test the capacity of the shells to prevent a trapped virus to undergo interactions with surfaces, in vitro virus blocking assays were performed with HBV-binding antibodies immobilized on a solid surface (FIG. 5G). The extent of HBV core particle binding to the surface was quantified via binding of an orthogonal HBV core-specific reporter antibody coupled to horseradish peroxidase (HRP). Residual HBV core particles that are bound to the surface are detected via HRP catalyzed production of a colorimetric signal. In the presence of the virus-engulfing shells (half T=1 shells), virus interactions with the surface were blocked up to 99% (FIG. 5G, bottom), thus confirming the interaction-inhibiting capacity of the shells. Control experiments with shells lacking HBV trapping antibody resulted in minimal virus blocking compared to the signal generated by naked HBV core particles that represent baseline 0% virus blocking.

HBV core particles directly incubated with antibodies, but without any shells present, were negligibly blocked from binding the surface. This finding indicates that the antibodies by themselves do not fully passivate the HBV capsid surface even though they were added at 400-fold excess over HBV particles. However, in contrast, when using the shells functionalized with on average as few as five antibodies, a virus blocking efficiency of greater than 80% was achieved. The blocking was nearly complete (up to 99%) when using more than five antibodies in the shells. The data thus shows that the shell-trapping method can be highly effective even when only a handful of physical interactions are formed between the virus surface and surrounding shell. The data indicates that the shells, and not the antibodies used for holding the virus inside the shell, shield the virus from its exterior by steric occlusion.

### Virus Neutralization in Human Cells

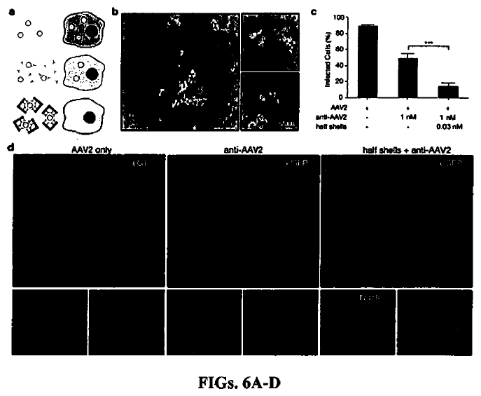

The neutralization capacity of the DNA-origami half octahedron shells was tested using adeno-associated virus serotype 2 (AAV2) (Wang et al., “Adeno-Associated Virus Vector as a Platform for Gene Therapy Delivery,” Nat. Rev. Drug Discov. 18:358-378 (2019), which is hereby incorporated by reference in its entirety) virions carrying an enhanced green fluorescent protein (eGFP) expression cassette (Guo et al., “Rapid AAV-Neutralizing Antibody Determination with a Cell-Binding Assay,” Mol. Ther. Methods Clin. Dev. 13:40-46 (2019), which is hereby incorporated by reference in its entirety) using both microscopy and flow cytometry (FIG. 6A). DNA shells were stabilized with UV point welding and PEG-oligolysine/oligolysine as described above. AAV2 particles were successfully trapped in DNA half shells functionalized with anti-AAV2 antibody in the shell interior, in serum in the presence of bovine serum albumin (BSA) as seen by direct imaging with TEM (FIG. 6B). Since AAV belongs to a completely different virus family than HBV, this data also establishes the modularity of the shell: by swapping out the virus-binding moieties one can trap different types of viruses.

The efficacy of virus neutralization was quantified by determining the dose response curves for DNA half-shells functionalized with on average 36 anti-AAV2 antibodies per half-shell and free anti-AAV2 antibodies as reference. The number of eGFP positive cells served as a readout for infection efficacy using flow cytometry analysis. The DNA half shells neutralized AAV2 with an estimated half maximal inhibitory concentration (IC₅₀) of ˜0.3 nM. At the conditions used, the IC₅₀ corresponded to approximately 2.5 half-shells per infectious virus particle. The DNA half shells had increased neutralization capacity compared to the activity of the free anti-AAV2 (FIGS. 6C-D). This neutralization enhancement is best appreciated in fluorescence microscopy images (FIG. 6D), where few eGFP positive cells remain in the samples with AAV2-trapping DNA half shells, whereas many eGFP positive cells appear in samples exposed to the identical dose of anti-AAV2 antibodies free in solution. This experiment demonstrates that the shells function in physiological conditions with live cells. It also shows that the shells can further augment the already quite potent neutralization capabilities of the anti-AAV2 antibodies. As above with the in vitro HBV blocking experiments in FIGS. 4A-K, the enhanced neutralization suggests that the shells trap viruses in a multivalent fashion and that the shell material additionally contributes as a viral-surface occluding agent.

It was also investigated whether the DNA-origami half shells without any conjugated antibody had an effect. A low but non-negligible neutralization activity was found at the highest origami concentration tested. This activity likely arises from electrostatic interactions between the PEG-oligolysine/oligolysine coated DNA-shells, and the AAV2 particles. Finally, it was tested if exposure to the DNA half shells had any effect on cell viability, and no significant effect across any of the concentrations used in this study was found.

### DISCUSSION

The experimental work described herein demonstrates the idea that trapping viruses in shells can decrease the viral load in acute viral infections by preventing viruses from undergoing host cell interactions. The virus trapping concept was tested successfully with HBV core and AAV2 virus particles. Near complete inactivation was achieved by engulfing HBV in a surrounding shell in vitro and it was also shown to effectively block AAV from infecting live cells. Due to the modularity of the DNA shells, other virus binders could be used. For example, host receptor domains or peptides known to be targeted by a viral pathogen and DNA/RNA aptamers could be conjugated to the shells. One of the design solutions, the half T=1 shell, featured 90 sites for anchoring virus-binding moieties in the interior cavity. This high level of multivalency will be particularly useful for trapping pathogens for which only low-affinity binders are available. Multiple different antibodies could also be combined to achieve higher specificity against a single target or against a plurality of targets.

The icosahedral shells are made of DNA, which is durable, available commercially, and easily functionalized and modified. The components needed for the shells can be mass-produced biotechnologically (Praetorius et al., “Biotechnological Mass Production of DNA Origami,” Nature 552:84-87 (2017), which is hereby incorporated by reference in its entirety). Using DNA-based agents can potentially circumvent neutralization, phagocytosis, and degradation by pathways of the innate and adaptive immune system targeting protein structures. It is expected that the shells described herein are largely non-toxic because they do not target any enzymes of the host metabolism as many current antivirals do. Beyond the proposed application as virus traps, the programmable icosahedral canvas system also offers opportunities to create antigen-carriers for vaccination, DNA or RNA carriers for gene therapy or gene modification, drug delivery vehicles, and protective storage containers (see FIGS. 8A-D for cargo loading examples).

### Methods

### Self-Assembly of Shell Subunits

All self-assembly experiments were performed in standardized “folding buffers” containing x mM MgCl₂ in addition to 5 mM Tris Base, 1 mM EDTA and 5 mM NaCl at pH 8 (FoBx). Single-scaffold-chain DNA-origami objects were self-assembled in one-pot folding reactions containing 50 nM scaffold DNA and 200 nM of each staple strand. The individual scaffolds were produced as described previously (Engelhardt et al., “Custom-Size, Functional, and Durable DNA Origami with Design-Specific Scaffolds,” ACS Nano (2019); Kick et al., “Efficient Production of Single-Stranded Phage DNA as Scaffolds for DNA Origami,” Nano Letters (2015); which are hereby incorporated by reference in their entirety). Folding buffer (FoB20) was used with x=20 mM MgCl₂. All reaction mixtures were subjected to thermal annealing ramps as detailed in Table 1 in Tetrad (Bio-Rad) thermal cycling devices. Staple strands were purchased from IDT (Integrated DNA Technologies).

TABLE 1
Temperature Ramps and Scaffold Molecules Used
for Self-Assembly of Shell Building Blocks

| | Denaturation | | | |
|---|---|---|---|---|
| | Phase Temperature | Temperature | Storage | |
| Object | (15 min) (° C.) | Ramp (1° C./1 h) | Temp. (° C.) | Scaffold |
| T_octa | 65 | 60-56° C. | 20 | M13 8064 |
| T = 1 | 65 | 58-54° C. | 20 | M13 8064 |
| T = 1 −5° | 65 | 58-54° C. | 20 | M13 8064 |
| T = 1 +5° | 65 | 58-54° C. | 20 | M13 8064 |
| T = 3 | 65 | 54-52° C. | 20 | M13 8064 |
| T = 4_iso | 65 | 56-54° C. | 20 | M13 8064 |
| T = 4_equi | 65 | 58-54° C. | 20 | M13 8064 |
| T = 9_pent | 65 | 56-52° C. | 20 | M13 7249 |
| T = 9_hex1 | 65 | 56-52° C. | 20 | M13 8064 |
| T = 9_hex2 | 65 | 58-54° C. | 20 | M13 8064 |
| T_octa_half | 65 | 58-54° C. | 20 | M13 8064 |
| T_pent | 65 | 58-54° C. | 20 | M13 8064 |
| T = 1 (⅔ triangles) | | | | |
| T_ring | 65 | 56-52° C. | 20 | M13 8064 |
| T = 1 (2 triangles) | | | | |
| T_ring1 | 65 | 56-52° C. | 20 | M13 8064 |
| T = 1 (3 triangles) | | | | |
| T_ring2 | 65 | 56-52° C. | 20 | M13 8064 |
| T = 1 (3 triangles) | | | | |
| triangular | 65 | 60-44° C. | 20 | M13 8064 |
| brick | | | | |

The scaffold nucleotide sequences of Table 1 are as follows:

M13 8064 (SEQ ID NO: 1):
GGCAATGACCTGATAGCCTTTGTAGATCTCTCAAAAATAGCTACCCTCTCCGGCATTAATTTATCAGCTA
GAACGGTTGAATATCATATTGATGGTGATTTGACTGTCTCCGGCCTTTCTCACCCTTTTGAATCTTTACC
TACACATTACTCAGGCATTGCATTTAAAATATATGAGGGTTCTAAAAATTTTTATCCTTGCGTTGAAATA
AAGGCTTCTCCCGCAAAAGTATTACAGGGTCATAATGTTTTTGGTACAACCGATTTAGCTTTATGCTCTG
AGGCTTTATTGCTTAATTTTGCTAATTCTTTGCCTTGCCTGTATGATTTATTGGATGTTAATGCTACTAC
TATTAGTAGAATTGATGCCACCTTTTCAGCTCGCGCCCCAAATGAAAATATAGCTAAACAGGTTATTGAC
CATTTGCGAAATGTATCTAATGGTCAAACTAAATCTACTCGTTCGCAGAATTGGGAATCAACTGTTATAT
GGAATGAAACTTCCAGACACCGTACTTTAGTTGCATATTTAAAACATGTTGAGCTACAGCATTATATTCA
GCAATTAAGCTCTAAGCCATCCGCAAAAATGACCTCTTATCAAAAGGAGCAATTAAAGGTACTCTCTAAT
CCTGACCTGTTGGAGTTTGCTTCCGGTCTGGTTCGCTTTGAAGCTCGAATTAAAACGCGATATTTGAAGT
CTTTCGGGCTTCCTCTTAATCTTTTTGATGCAATCCGCTTTGCTTCTGACTATAATAGTCAGGGTAAAGA
CCTGATTTTTGATTTATGGTCATTCTCGTTTTCTGAACTGTTTAAAGCATTTGAGGGGGATTCAATGAAT
ATTTATGACGATTCCGCAGTATTGGACGCTATCCAGTCTAAACATTTTACTATTACCCCCTCTGGCAAAA
CTTCTTTTGCAAAAGCCTCTCGCTATTTTGGTTTTTATCGTCGTCTGGTAAACGAGGGTTATGATAGTGT
TGCTCTTACTATGCCTCGTAATTCCTTTTGGCGTTATGTATCTGCATTAGTTGAATGTGGTATTCCTAAA
TCTCAACTGATGAATCTTTCTACCTGTAATAATGTTGTTCCGTTAGTTCGTTTTATTAACGTAGATTTTT
CTTCCCAACGTCCTGACTGGTATAATGAGCCAGTTCTTAAAATCGCATAAGGTAATTCACAATGATTAAA
GTTGAAATTAAACCATCTCAAGCCCAATTTACTACTCGTTCTGGTGTTTCTCGTCAGGGCAAGCCTTATT
CACTGAATGAGCAGCTTTGTTACGTTGATTTGGGTAATGAATATCCGGTTCTTGTCAAGATTACTCTTGA
TGAAGGTCAGCCAGCCTATGCGCCTGGTCTGTACACCGTTCATCTGTCCTCTTTCAAAGTTGGTCAGTTC
GGTTCCCTTATGATTGACCGTCTGCGCCTCGTTCCGGCTAAGTAACATGGAGCAGGTCGCGGATTTCGAC
ACAATTTATCAGGCGATGATACAAATCTCCGTTGTACTTTGTTTCGCGCTTGGTATAATCGCTGGGGGTC
AAAGATGAGTGTTTTAGTGTATTCTTTTGCCTCTTTCGTTTTAGGTTGGTGCCTTCGTAGTGGCATTACG
TATTTTACCCGTTTAATGGAAACTTCCTCATGAAAAAGTCTTTAGTCCTCAAAGCCTCTGTAGCCGTTGC
TACCCTCGTTCCGATGCTGTCTTTCGCTGCTGAGGGTGACGATCCCGCAAAAGCGGCCTTTAACTCCCTG
CAAGCCTCAGCGACCGAATATATCGGTTATGCGTGGGCGATGGTTGTTGTCATTGTCGGCGCAACTATCG
GTATCAAGCTGTTTAAGAAATTCACCTCGAAAGCAAGCTGATAAACCGATACAATTAAAGGCTCCTTTTG
GAGCCTTTTTTTTGGAGATTTTCAACGTGAAAAAATTATTATTCGCAATTCCTTTAGTTGTTCCTTTCTA
TTCTCACTCCGCTGAAACTGTTGAAAGTTGTTTAGCAAAATCCCATACAGAAAATTCATTTACTAACGTC
TGGAAAGACGACAAAACTTTAGATCGTTACGCTAACTATGAGGGCTGTCTGTGGAATGCTACAGGCGTTG
TAGTTTGTACTGGTGACGAAACTCAGTGTTACGGTACATGGGTTCCTATTGGGCTTGCTATCCCTGAAAA
TGAGGGTGGTGGCTCTGAGGGTGGCGGTTCTGAGGGTGGCGGTTCTGAGGGTGGCGGTACTAAACCTCCT
GAGTACGGTGATACACCTATTCCGGGCTATACTTATATCAACCCTCTCGACGGCACTTATCCGCCTGGTA
CTGAGCAAAACCCCGCTAATCCTAATCCTTCTCTTGAGGAGTCTCAGCCTCTTAATACTTTCATGTTTCA
GAATAATAGGTTCCGAAATAGGCAGGGGGCATTAACTGTTTATACGGGCACTGTTACTCAAGGCACTGAC
CCCGTTAAAACTTATTACCAGTACACTCCTGTATCATCAAAAGCCATGTATGACGCTTACTGGAACGGTA
AATTCAGAGACTGCGCTTTCCATTCTGGCTTTAATGAGGATTTATTTGTTTGTGAATATCAAGGCCAATC
GTCTGACCTGCCTCAACCTCCTGTCAATGCTGGCGGCGGCTCTGGTGGTGGTTCTGGTGGCGGCTCTGAG
GGTGGTGGCTCTGAGGGTGGCGGTTCTGAGGGTGGCGGCTCTGAGGGAGGCGGTTCCGGTGGTGGCTCTG
GTTCCGGTGATTTTGATTATGAAAAGATGGCAAACGCTAATAAGGGGGCTATGACCGAAAATGCCGATGA
AAACGCGCTACAGTCTGACGCTAAAGGCAAACTTGATTCTGTCGCTACTGATTACGGTGCTGCTATCGAT
GGTTTCATTGGTGACGTTTCCGGCCTTGCTAATGGTAATGGTGCTACTGGTGATTTTGCTGGCTCTAATT
CCCAAATGGCTCAAGTCGGTGACGGTGATAATTCACCTTTAATGAATAATTTCCGTCAATATTTACCTTC
CCTCCCTCAATCGGTTGAATGTCGCCCTTTTGTCTTTGGCGCTGGTAAACCATATGAATTTTCTATTGAT
TGTGACAAAATAAACTTATTCCGTGGTGTCTTTGCGTTTCTTTTATATGTTGCCACCTTTATGTATGTAT
TTTCTACGTTTGCTAACATACTGCGTAATAAGGAGTCTTAATCATGCCAGTTCTTTTGGGTATTCCGTTA
TTATTGCGTTTCCTCGGTTTCCTTCTGGTAACTTTGTTCGGCTATCTGCTTACTTTTCTTAAAAAGGGCT
TCGGTAAGATAGCTATTGCTATTTCATTGTTTCTTGCTCTTATTATTGGGCTTAACTCAATTCTTGTGGG
TTATCTCTCTGATATTAGCGCTCAATTACCCTCTGACTTTGTTCAGGGTGTTCAGTTAATTCTCCCGTCT
AATGCGCTTCCCTGTTTTTATGTTATTCTCTCTGTAAAGGCTGCTATTTTCATTTTTGACGTTAAACAAA
AAATCGTTTCTTATTTGGATTGGGATAAATAATATGGCTGTTTATTTTGTAACTGGCAAATTAGGCTCTG
GAAAGACGCTCGTTAGCGTTGGTAAGATTCAGGATAAAATTGTAGCTGGGTGCAAAATAGCAACTAATCT
TGATTTAAGGCTTCAAAACCTCCCGCAAGTCGGGAGGTTCGCTAAAACGCCTCGCGTTCTTAGAATACCG
GATAAGCCTTCTATATCTGATTTGCTTGCTATTGGGCGCGGTAATGATTCCTACGATGAAAATAAAAACG
GCTTGCTTGTTCTCGATGAGTGCGGTACTTGGTTTAATACCCGTTCTTGGAATGATAAGGAAAGACAGCC
GATTATTGATTGGTTTCTACATGCTCGTAAATTAGGATGGGATATTATTTTTCTTGTTCAGGACTTATCT
ATTGTTGATAAACAGGCGCGTTCTGCATTAGCTGAACATGTTGTTTATTGTCGTCGTCTGGACAGAATTA
CTTTACCTTTTGTCGGTACTTTATATTCTCTTATTACTGGCTCGAAAATGCCTCTGCCTAAATTACATGT
TGGCGTTGTTAAATATGGCGATTCTCAATTAAGCCCTACTGTTGAGCGTTGGCTTTATACTGGTAAGAAT
TTGTATAACGCATATGATACTAAACAGGCTTTTTCTAGTAATTATGATTCCGGTGTTTATTCTTATTTAA
CGCCTTATTTATCACACGGTCGGTATTTCAAACCATTAAATTTAGGTCAGAAGATGAAATTAACTAAAAT
ATATTTGAAAAAGTTTTCTCGCGTTCTTTGTCTTGCGATTGGATTTGCATCAGCATTTACATATAGTTAT
ATAACCCAACCTAAGCCGGAGGTTAAAAAGGTAGTCTCTCAGACCTATGATTTTGATAAATTCACTATTG
ACTCTTCTCAGCGTCTTAATCTAAGCTATCGCTATGTTTTCAAGGATTCTAAGGGAAAATTAATTAATAG
CGACGATTTACAGAAGCAAGGTTATTCACTCACATATATTGATTTATGTACTGTTTCCATTAAAAAAGGT
AATTCAAATGAAATTGTTAAATGTAATTAATTTTGTTTTCTTGATGTTTGTTTCATCATCTTCTTTTGCT
CAGGTAATTGAAATGAATAATTCGCCTCTGCGCGATTTTGTAACTTGGTATTCAAAGCAATCAGGCGAAT
CCGTTATTGTTTCTCCCGATGTAAAAGGTACTGTTACTGTATATTCATCTGACGTTAAACCTGAAAATCT
ACGCAATTTCTTTATTTCTGTTTTACGTGCAAATAATTTTGATATGGTAGGTTCTAACCCTTCCATTATT
CAGAAGTATAATCCAAACAATCAGGATTATATTGATGAATTGCCATCATCTGATAATCAGGAATATGATG
ATAATTCCGCTCCTTCTGGTGGTTTCTTTGTTCCGCAAAATGATAATGTTACTCAAACTTTTAAAATTAA
TAACGTTCGGGCAAAGGATTTAATACGAGTTGTCGAATTGTTTGTAAAGTCTAATACTTCTAAATCCTCA
AATGTATTATCTATTGACGGCTCTAATCTATTAGTTGTTAGTGCTCCTAAAGATATTTTAGATAACCTTC
CTCAATTCCTTTCAACTGTTGATTTGCCAACTGACCAGATATTGATTGAGGGTTTGATATTTGAGGTTCA
GCAAGGTGATGCTTTAGATTTTTCATTTGCTGCTGGCTCTCAGCGTGGCACTGTTGCAGGCGGTGTTAAT
ACTGACCGCCTCACCTCTGTTTTATCTTCTGCTGGTGGTTCGTTCGGTATTTTTAATGGCGATGTTTTAG
GGCTATCAGTTCGCGCATTAAAGACTAATAGCCATTCAAAAATATTGTCTGTGCCACGTATTCTTACGCT
TTCAGGTCAGAAGGGTTCTATCTCTGTTGGCCAGAATGTCCCTTTTATTACTGGTCGTGTGACTGGTGAA
TCTGCCAATGTAAATAATCCATTTCAGACGATTGAGCGTCAAAATGTAGGTATTTCCATGAGCGTTTTTC
CTGTTGCAATGGCTGGCGGTAATATTGTTCTGGATATTACCAGCAAGGCCGATAGTTTGAGTTCTTCTAC
TCAGGCAAGTGATGTTATTACTAATCAAAGAAGTATTGCTACAACGGTTAATTTGCGTGATGGACAGACT
CTTTTACTCGGTGGCCTCACTGATTATAAAAACACTTCTCAGGATTCTGGCGTACCGTTCCTGTCTAAAA
TCCCTTTAATCGGCCTCCTGTTTAGCTCCCGCTCTGATTCTAACGAGGAAAGCACGTTATACGTGCTCGT
CAAAGCAACCATAGTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCACC
TCGACCCCAAAAAACTTGATTTGGGTGATGGTTCACGTAGTGGGCCATCGCCCTGATAGACGGTTTTTCG
CCCTTTGACGTTGGAGTCCACGTTCTTTAATAGTGGACTCTTGTTCCAAACTGGAACAACACTCAACCCT
ATCTCGGGCTATTCTTTTGATTTATAAGGGATTTTGCCGATTTCGGAACCACCATCAAACAGGATTTTCG
CCTGCTGGGGCAAACCAGCGTGGACCGCTTGCTGCAACTCTCTCAGGGCCAGGCGGTGAAGGGCAATCAG
CTGTTGCCCGTCTCACTGGTGAAAAGAAAAACCACCCTGGCGCCCAATACGCAAACCGCCTCTCCCCGCG
CGTTGGCCGATTCATTAATGCAGCTGGCACGACAGGTTTCCCGACTGGAAAGCGGGCAGTGAGCGCAACG
CAATTAATGTGAGTTAGCTCACTCATTAGGCACCCCAGGCTTTACACTTTATGCTTCCGGCTCGTATGTT
GTGTGGAATTGTGAGCGGATAACAATTTCACACAGGAAACAGCTATGACCATGATTACGAATTCGAGCTC
GGTACCCGGGGATCCTCAACTGTGAGGAGGCTCACGGACGCGAAGAACAGGCACGCGTGCTGGCAGAAAC
CCCCGGTATGACCGTGAAAACGGCCCGCCGCATTCTGGCCGCAGCACCACAGAGTGCACAGGCGCGCAGT
GACACTGCGCTGGATCGTCTGATGCAGGGGGCACCGGCACCGCTGGCTGCAGGTAACCCGGCATCTGATG
CCGTTAACGATTTGCTGAACACACCAGTGTAAGGGATGTTTATGACGAGCAAAGAAACCTTTACCCATTA
CCAGCCGCAGGGCAACAGTGACCCGGCTCATACCGCAACCGCGCCCGGCGGATTGAGTGCGAAAGCGCCT
GCAATGACCCCGCTGATGCTGGACACCTCCAGCCGTAAGCTGGTTGCGTGGGATGGCACCACCGACGGTG
CTGCCGTTGGCATTCTTGCGGTTGCTGCTGACCAGACCAGCACCACGCTGACGTTCTACAAGTCCGGCAC
GTTCCGTTATGAGGATGTGCTCTGGCCGGAGGCTGCCAGCGACGAGACGAAAAAACGGACCGCGTTTGCC
GGAACGGCAATCAGCATCGTTTAACTTTACCCTTCATCACTAAAGGCCGCCTGTGCGGCTTTTTTTACGG
GATTTTTTTATGTCGATGTACACAACCGCCCAACTGCTGGCGGCAAATGAGCAGAAATTTAAGTTTGATC
CGCTGTTTCTGCGTCTCTTTTTCCGTGAGAGCTATCCCTTCACCACGGAGAAAGTCTATCTCTCACAAAT
TCCGGGACTGGTAAACATGGCGCTGTACGTTTCGCCGATTGTTTCCGGTGAGGTTATCCGTTCCCGTGGC
GGCTCCACCTCTGAAAGCTTGGCACTGGCCGTCGTTTTACAACGTCGTGACTGGGAAAACCCTGGCGTTA
CCCAACTTAATCGCCTTGCAGCACATCCCCCTTTCGCCAGCTGGCGTAATAGCGAAGAGGCCCGCACCGA
TCGCCCTTCCCAACAGTTGCGCAGCCTGAATGGCGAATGGCGCTTTGCCTGGTTTCCGGCACCAGAAGCG
GTGCCGGAAAGCTGGCTGGAGTGCGATCTTCCTGAGGCCGATACTGTCGTCGTCCCCTCAAACTGGCAGA
TGCACGGTTACGATGCGCCCATCTACACCAACGTGACCTATCCCATTACGGTCAATCCGCCGTTTGTTCC
CACGGAGAATCCGACGGGTTGTTACTCGCTCACATTTAATGTTGATGAAAGCTGGCTACAGGAAGGCCAG
ACGCGAATTATTTTTGATGGCGTTCCTATTGGTTAAAAAATGAGCTGATTTAACAAAAATTTAATGCGAA
TTTTAACAAAATATTAACGTTTACAATTTAAATATTTGCTTATACAATCTTCCTGTTTTTGGGGCTTTTC
TGATTATCAACCGGGGTACATATGATTGACATGCTAGTTTTACGATTACCGTTCATCGATTCTCTTGTTT
GCTCCAGACTCTCA
See Engelhardt et al., “Custom-Size, Functional, and Durable DNA Origami with Design-
Specific Scaffolds,” ACS Nano 13(5): 5015-5027 (2019), which is hereby incorporated by
reference in its entirety.
M13 7249 (SEQ ID NO: 2):
TGATAGACGGTTTTTCGCCCTTTGACGTTGGAGTCCACGTTCTTTAATAGTGGACTCTTGTTCCAAACTG
GAACAACACTCAACCCTATCTCGGGCTATTCTTTTGATTTATAAGGGATTTTGCCGATTTCGGAACCACC
ATCAAACAGGATTTTCGCCTGCTGGGGCAAACCAGCGTGGACCGCTTGCTGCAACTCTCTCAGGGCCAGG
CGGTGAAGGGCAATCAGCTGTTGCCCGTCTCACTGGTGAAAAGAAAAACCACCCTGGCGCCCAATACGCA
AACCGCCTCTCCCCGCGCGTTGGCCGATTCATTAATGCAGCTGGCACGACAGGTTTCCCGACTGGAAAGC
GGGCAGTGAGCGCAACGCAATTAATGTGAGTTAGCTCACTCATTAGGCACCCCAGGCTTTACACTTTATG
CTTCCGGCTCGTATGTTGTGTGGAATTGTGAGCGGATAACAATTTCACACAGGAAACAGCTATGACCATG
ATTACGAATTCGAGCTCGGTACCCGGGGATCCTCTAGAGTCGACCTGCAGGCATGCAAGCTTGGCACTGG
CCGTCGTTTTACAACGTCGTGACTGGGAAAACCCTGGCGTTACCCAACTTAATCGCCTTGCAGCACATCC
CCCTTTCGCCAGCTGGCGTAATAGCGAAGAGGCCCGCACCGATCGCCCTTCCCAACAGTTGCGCAGCCTG
AATGGCGAATGGCGCTTTGCCTGGTTTCCGGCACCAGAAGCGGTGCCGGAAAGCTGGCTGGAGTGCGATC
TTCCTGAGGCCGATACTGTCGTCGTCCCCTCAAACTGGCAGATGCACGGTTACGATGCGCCCATCTACAC
CAACGTGACCTATCCCATTACGGTCAATCCGCCGTTTGTTCCCACGGAGAATCCGACGGGTTGTTACTCG
CTCACATTTAATGTTGATGAAAGCTGGCTACAGGAAGGCCAGACGCGAATTATTTTTGATGGCGTTCCTA
TTGGTTAAAAAATGAGCTGATTTAACAAAAATTTAATGCGAATTTTAACAAAATATTAACGTTTACAATT
TAAATATTTGCTTATACAATCTTCCTGTTTTTGGGGCTTTTCTGATTATCAACCGGGGTACATATGATTG
ACATGCTAGTTTTACGATTACCGTTCATCGATTCTCTTGTTTGCTCCAGACTCTCAGGCAATGACCTGAT
AGCCTTTGTAGATCTCTCAAAAATAGCTACCCTCTCCGGCATTAATTTATCAGCTAGAACGGTTGAATAT
CATATTGATGGTGATTTGACTGTCTCCGGCCTTTCTCACCCTTTTGAATCTTTACCTACACATTACTCAG
GCATTGCATTTAAAATATATGAGGGTTCTAAAAATTTTTATCCTTGCGTTGAAATAAAGGCTTCTCCCGC
AAAAGTATTACAGGGTCATAATGTTTTTGGTACAACCGATTTAGCTTTATGCTCTGAGGCTTTATTGCTT
AATTTTGCTAATTCTTTGCCTTGCCTGTATGATTTATTGGATGTTAATGCTACTACTATTAGTAGAATTG
ATGCCACCTTTTCAGCTCGCGCCCCAAATGAAAATATAGCTAAACAGGTTATTGACCATTTGCGAAATGT
ATCTAATGGTCAAACTAAATCTACTCGTTCGCAGAATTGGGAATCAACTGTTATATGGAATGAAACTTCC
AGACACCGTACTTTAGTTGCATATTTAAAACATGTTGAGCTACAGCATTATATTCAGCAATTAAGCTCTA
AGCCATCCGCAAAAATGACCTCTTATCAAAAGGAGCAATTAAAGGTACTCTCTAATCCTGACCTGTTGGA
GTTTGCTTCCGGTCTGGTTCGCTTTGAAGCTCGAATTAAAACGCGATATTTGAAGTCTTTCGGGCTTCCT
CTTAATCTTTTTGATGCAATCCGCTTTGCTTCTGACTATAATAGTCAGGGTAAAGACCTGATTTTTGATT
TATGGTCATTCTCGTTTTCTGAACTGTTTAAAGCATTTGAGGGGGATTCAATGAATATTTATGACGATTC
CGCAGTATTGGACGCTATCCAGTCTAAACATTTTACTATTACCCCCTCTGGCAAAACTTCTTTTGCAAAA
GCCTCTCGCTATTTTGGTTTTTATCGTCGTCTGGTAAACGAGGGTTATGATAGTGTTGCTCTTACTATGC
CTCGTAATTCCTTTTGGCGTTATGTATCTGCATTAGTTGAATGTGGTATTCCTAAATCTCAACTGATGAA
TCTTTCTACCTGTAATAATGTTGTTCCGTTAGTTCGTTTTATTAACGTAGATTTTTCTTCCCAACGTCCT
GACTGGTATAATGAGCCAGTTCTTAAAATCGCATAAGGTAATTCACAATGATTAAAGTTGAAATTAAACC
ATCTCAAGCCCAATTTACTACTCGTTCTGGTGTTTCTCGTCAGGGCAAGCCTTATTCACTGAATGAGCAG
CTTTGTTACGTTGATTTGGGTAATGAATATCCGGTTCTTGTCAAGATTACTCTTGATGAAGGTCAGCCAG
CCTATGCGCCTGGTCTGTACACCGTTCATCTGTCCTCTTTCAAAGTTGGTCAGTTCGGTTCCCTTATGAT
TGACCGTCTGCGCCTCGTTCCGGCTAAGTAACATGGAGCAGGTCGCGGATTTCGACACAATTTATCAGGC
GATGATACAAATCTCCGTTGTACTTTGTTTCGCGCTTGGTATAATCGCTGGGGGTCAAAGATGAGTGTTT
TAGTGTATTCTTTTGCCTCTTTCGTTTTAGGTTGGTGCCTTCGTAGTGGCATTACGTATTTTACCCGTTT
AATGGAAACTTCCTCATGAAAAAGTCTTTAGTCCTCAAAGCCTCTGTAGCCGTTGCTACCCTCGTTCCGA
TGCTGTCTTTCGCTGCTGAGGGTGACGATCCCGCAAAAGCGGCCTTTAACTCCCTGCAAGCCTCAGCGAC
CGAATATATCGGTTATGCGTGGGCGATGGTTGTTGTCATTGTCGGCGCAACTATCGGTATCAAGCTGTTT
AAGAAATTCACCTCGAAAGCAAGCTGATAAACCGATACAATTAAAGGCTCCTTTTGGAGCCTTTTTTTTG
GAGATTTTCAACGTGAAAAAATTATTATTCGCAATTCCTTTAGTTGTTCCTTTCTATTCTCACTCCGCTG
AAACTGTTGAAAGTTGTTTAGCAAAATCCCATACAGAAAATTCATTTACTAACGTCTGGAAAGACGACAA
AACTTTAGATCGTTACGCTAACTATGAGGGCTGTCTGTGGAATGCTACAGGCGTTGTAGTTTGTACTGGT
GACGAAACTCAGTGTTACGGTACATGGGTTCCTATTGGGCTTGCTATCCCTGAAAATGAGGGTGGTGGCT
CTGAGGGTGGCGGTTCTGAGGGTGGCGGTTCTGAGGGTGGCGGTACTAAACCTCCTGAGTACGGTGATAC
ACCTATTCCGGGCTATACTTATATCAACCCTCTCGACGGCACTTATCCGCCTGGTACTGAGCAAAACCCC
GCTAATCCTAATCCTTCTCTTGAGGAGTCTCAGCCTCTTAATACTTTCATGTTTCAGAATAATAGGTTCC
GAAATAGGCAGGGGGCATTAACTGTTTATACGGGCACTGTTACTCAAGGCACTGACCCCGTTAAAACTTA
TTACCAGTACACTCCTGTATCATCAAAAGCCATGTATGACGCTTACTGGAACGGTAAATTCAGAGACTGC
GCTTTCCATTCTGGCTTTAATGAGGATTTATTTGTTTGTGAATATCAAGGCCAATCGTCTGACCTGCCTC
AACCTCCTGTCAATGCTGGCGGCGGCTCTGGTGGTGGTTCTGGTGGCGGCTCTGAGGGTGGTGGCTCTGA
GGGTGGCGGTTCTGAGGGTGGCGGCTCTGAGGGAGGCGGTTCCGGTGGTGGCTCTGGTTCCGGTGATTTT
GATTATGAAAAGATGGCAAACGCTAATAAGGGGGCTATGACCGAAAATGCCGATGAAAACGCGCTACAGT
CTGACGCTAAAGGCAAACTTGATTCTGTCGCTACTGATTACGGTGCTGCTATCGATGGTTTCATTGGTGA
CGTTTCCGGCCTTGCTAATGGTAATGGTGCTACTGGTGATTTTGCTGGCTCTAATTCCCAAATGGCTCAA
GTCGGTGACGGTGATAATTCACCTTTAATGAATAATTTCCGTCAATATTTACCTTCCCTCCCTCAATCGG
TTGAATGTCGCCCTTTTGTCTTTGGCGCTGGTAAACCATATGAATTTTCTATTGATTGTGACAAAATAAA
CTTATTCCGTGGTGTCTTTGCGTTTCTTTTATATGTTGCCACCTTTATGTATGTATTTTCTACGTTTGCT
AACATACTGCGTAATAAGGAGTCTTAATCATGCCAGTTCTTTTGGGTATTCCGTTATTATTGCGTTTCCT
CGGTTTCCTTCTGGTAACTTTGTTCGGCTATCTGCTTACTTTTCTTAAAAAGGGCTTCGGTAAGATAGCT
ATTGCTATTTCATTGTTTCTTGCTCTTATTATTGGGCTTAACTCAATTCTTGTGGGTTATCTCTCTGATA
TTAGCGCTCAATTACCCTCTGACTTTGTTCAGGGTGTTCAGTTAATTCTCCCGTCTAATGCGCTTCCCTG
TTTTTATGTTATTCTCTCTGTAAAGGCTGCTATTTTCATTTTTGACGTTAAACAAAAAATCGTTTCTTAT
TTGGATTGGGATAAATAATATGGCTGTTTATTTTGTAACTGGCAAATTAGGCTCTGGAAAGACGCTCGTT
AGCGTTGGTAAGATTCAGGATAAAATTGTAGCTGGGTGCAAAATAGCAACTAATCTTGATTTAAGGCTTC
AAAACCTCCCGCAAGTCGGGAGGTTCGCTAAAACGCCTCGCGTTCTTAGAATACCGGATAAGCCTTCTAT
ATCTGATTTGCTTGCTATTGGGCGCGGTAATGATTCCTACGATGAAAATAAAAACGGCTTGCTTGTTCTC
GATGAGTGCGGTACTTGGTTTAATACCCGTTCTTGGAATGATAAGGAAAGACAGCCGATTATTGATTGGT
TTCTACATGCTCGTAAATTAGGATGGGATATTATTTTTCTTGTTCAGGACTTATCTATTGTTGATAAACA
GGCGCGTTCTGCATTAGCTGAACATGTTGTTTATTGTCGTCGTCTGGACAGAATTACTTTACCTTTTGTC
GGTACTTTATATTCTCTTATTACTGGCTCGAAAATGCCTCTGCCTAAATTACATGTTGGCGTTGTTAAAT
ATGGCGATTCTCAATTAAGCCCTACTGTTGAGCGTTGGCTTTATACTGGTAAGAATTTGTATAACGCATA
TGATACTAAACAGGCTTTTTCTAGTAATTATGATTCCGGTGTTTATTCTTATTTAACGCCTTATTTATCA
CACGGTCGGTATTTCAAACCATTAAATTTAGGTCAGAAGATGAAATTAACTAAAATATATTTGAAAAAGT
TTTCTCGCGTTCTTTGTCTTGCGATTGGATTTGCATCAGCATTTACATATAGTTATATAACCCAACCTAA
GCCGGAGGTTAAAAAGGTAGTCTCTCAGACCTATGATTTTGATAAATTCACTATTGACTCTTCTCAGCGT
CTTAATCTAAGCTATCGCTATGTTTTCAAGGATTCTAAGGGAAAATTAATTAATAGCGACGATTTACAGA
AGCAAGGTTATTCACTCACATATATTGATTTATGTACTGTTTCCATTAAAAAAGGTAATTCAAATGAAAT
TGTTAAATGTAATTAATTTTGTTTTCTTGATGTTTGTTTCATCATCTTCTTTTGCTCAGGTAATTGAAAT
GAATAATTCGCCTCTGCGCGATTTTGTAACTTGGTATTCAAAGCAATCAGGCGAATCCGTTATTGTTTCT
CCCGATGTAAAAGGTACTGTTACTGTATATTCATCTGACGTTAAACCTGAAAATCTACGCAATTTCTTTA
TTTCTGTTTTACGTGCAAATAATTTTGATATGGTAGGTTCTAACCCTTCCATTATTCAGAAGTATAATCC
AAACAATCAGGATTATATTGATGAATTGCCATCATCTGATAATCAGGAATATGATGATAATTCCGCTCCT
TCTGGTGGTTTCTTTGTTCCGCAAAATGATAATGTTACTCAAACTTTTAAAATTAATAACGTTCGGGCAA
AGGATTTAATACGAGTTGTCGAATTGTTTGTAAAGTCTAATACTTCTAAATCCTCAAATGTATTATCTAT
TGACGGCTCTAATCTATTAGTTGTTAGTGCTCCTAAAGATATTTTAGATAACCTTCCTCAATTCCTTTCA
ACTGTTGATTTGCCAACTGACCAGATATTGATTGAGGGTTTGATATTTGAGGTTCAGCAAGGTGATGCTT
TAGATTTTTCATTTGCTGCTGGCTCTCAGCGTGGCACTGTTGCAGGCGGTGTTAATACTGACCGCCTCAC
CTCTGTTTTATCTTCTGCTGGTGGTTCGTTCGGTATTTTTAATGGCGATGTTTTAGGGCTATCAGTTCGC
GCATTAAAGACTAATAGCCATTCAAAAATATTGTCTGTGCCACGTATTCTTACGCTTTCAGGTCAGAAGG
GTTCTATCTCTGTTGGCCAGAATGTCCCTTTTATTACTGGTCGTGTGACTGGTGAATCTGCCAATGTAAA
TAATCCATTTCAGACGATTGAGCGTCAAAATGTAGGTATTTCCATGAGCGTTTTTCCTGTTGCAATGGCT
GGCGGTAATATTGTTCTGGATATTACCAGCAAGGCCGATAGTTTGAGTTCTTCTACTCAGGCAAGTGATG
TTATTACTAATCAAAGAAGTATTGCTACAACGGTTAATTTGCGTGATGGACAGACTCTTTTACTCGGTGG
CCTCACTGATTATAAAAACACTTCTCAGGATTCTGGCGTACCGTTCCTGTCTAAAATCCCTTTAATCGGC
CTCCTGTTTAGCTCCCGCTCTGATTCTAACGAGGAAAGCACGTTATACGTGCTCGTCAAAGCAACCATAG
TACGCGCCCTGTAGCGGCGCATTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTACGGCACCTCGACCCCAAAAAA
CTTGATTTGGGTGATGGTTCACGTAGTGGGCCATCGCCC
See Stahl et al., “Facile and Scalable Preparation of Pure and Dense DNA Origami Solutions,”
Angewandte Chemie 53: 12735-12740 (2014), which is hereby incorporated by reference in its
entirety.

Staple sequences used in the construction of icosahedral shells formed by triangles, as described herein, are set forth in Tables 3-10 below.

### Purification of Shell Subunits and Self-Assembly of Shells

All shell subunits were purified using gel purification and, if necessary, concentrated with ultrafiltration (Amicon Ultra 500 μl with 100 kDa molecular weight cutoff) before self-assembling the subunits into shells. Both procedures were performed as previously described (Wagenbauer et al., “How we Make DNA Origami,” Chembiochem: A European Journal of Chemical Biology (2017), which is hereby incorporated by reference in its entirety) with the following alterations: for gel purification, 1.5% agarose gels containing 0.5×TBE and 5.5 mM MgCl₂ were used. For ultrafiltration, the same filter was filled with gel-purified sample multiple times (about 2-5 times, ˜400 μl every step) in order to increase the concentration of objects that are recovered from the filter. Before putting the filter upside down in a new filter tube, two washing steps were performed with 1×FoB5 (˜400 μl) to achieve well-defined buffer conditions for the shell assembly. To assemble the purified (and concentrated) shell subunits into shells, the subunit and MgCl₂ concentrations were adjusted by adding 1×FoB5 and 1.735 M MgCl₂ in suitable amounts. Typical subunit concentrations were in the range of 5 nM and up to 100 nM (for cryo-EM measurements, see Table 2). Typical MgCl₂ concentrations for shell self-assembly were in the range of 10-40 mM. Shell self-assembly was performed at 40° C. Reaction times were varied depending on the shell type (see FIG. 3A). Both, all shell subunits and assembled shells, can be stored at room temperature for several months.

### T=1 Shell Exterior Modification

The T=1 triangle and the triangular brick (FIG. 2C) were dimerized using single stranded DNA sticky ends protruding from the T=1 triangle. The protruding sequences contained three thymidines for flexibility plus 7 base long sequence motifs that were directly complementary to single stranded scaffold domains of the brick. Dimerization reactions were performed at room temperature overnight using a monomer concentration of 40 nM in the presence of 11 mM MgCl₂.

### Cargo Encapsulation In T=1 Shells

Nine staples of the T=1 shell subunits were modified by adding 16 bases on the 5′ ends. These nine modified staples and unmodified T=1 staples are folded with p8064 scaffold to produce T=1 triangles with nine ssDNA “handles” (FIG. 8A, left). The 16-base ssDNA handles are located on the shell-inward facing surface of the monomers. 8 of those 9 strands were oriented facing inwards towards the interior of the monomer and consequently may not have been accessible to the cargo. Single-stranded DNA cargo was prepared by attaching staple strands to the p8064 ssDNA circular scaffold with a 16 base-long overhang that was complementary to the handles on the shell subunits. An oligo containing a CY5 dye was also hybridized to the scaffold to enable fluorescence read-out by laser scanning of agarose gels (FIG. 8A, middle). In order to avoid having the unbound staples in cargo solution, which would passivate the monomers, 20 different staples were mixed with the scaffolds in 1:2 ratio. To anneal staples to the circular ssDNA, FOB15 buffer was used with a temperature ramp of 65° C. for 15 min, 60° C. to 44° C. for 1 h/1° C. To encapsulate gold nanoparticles, complementary handles of the monomer's handles were attached to the gold nanoparticles with a diameter of 30 nm (Cytodiagnostics, OligoREADY Gold Nanoparticle Conjugation Kit). A schematic and a negative stain TEM tomogram slice is shown in FIGS. 8B-C. To increase the visibility of the encapsulated circular ssDNA in TEM images, gold nanoparticles with a diameter of 20 nm (Cytodiagnostics, OligoREADY Gold Nanoparticle Conjugation Kit) were attached to the circular ssDNA scaffold (schematic and negative stain TEM are shown in FIGS. 8B-C, last images from the right). T=1 shells, with & without cargo were assembled in 1×FoB20 buffer at 40° C. for 3 days. Shell subunits were gel purified prior to assembly. Concentration of triangles was 16 nM. Concentration of cargo (of any type) was 0.8 nM.

### Half Shells and HBV Core Binding

Nine staples on the inside of the triangles were modified with handles with 26 single-stranded bases at the 5′ ends (SEQ ID NO:3): ‘GCAGTAGAGTAGGTAGAGATTAGGCA-oligonucleotide’. The triangles were purified and assembled as described above. Oligonucleotides complementary to the handle-sequence and modified with a thiol group at the 3′ end were coupled to the HBcore 17H7 antibody using a Sulfo-SMCC (Sulfosuccinimidyl-4-[N-maleimidomethyl]cyclohexane-1-carboxylate) cross-linker. The product was subsequently purified using the proFIRER from Dynamic Biosensors. The DNA modified antibodies were added to the assembled shells and incubated over night at 25° C. HBV core particles were incubated with the modified shells for 1-4 hours at 25° C. To assemble T=1 triangles around HBV core particles, the modified antibodies were added to single triangles. These triangles were then incubated with HBV core particles at a MgCl₂ concentration of 19 mM for one day.

### Shell Oligolysine Stabilization

The complete octahedral shells were assembled at 35 mM MgCl₂ and UV cross-linked as described in (Gerling et al., “Sequence-Programmable Covalent Bonding of Designed DNA Assemblies,” Sci. Adv. 4: eaau1157 (2018), which is hereby incorporated by reference in its entirety) for 1 h at 310 nm wavelength using the Asahi Spectra Xenon Light source 300 W MAX-303. The shells were incubated in a 0.6:1 ratio of N:P with a mixture of K₁₀ oligolysine and K₁₀-PEG_{5K} oligolysine (1:1) for 1 h at room temperature as similarly described in (Ponnuswamy et al., “Oligolysine-Based Coating Protects DNA Nanostructures from Low-Salt Denaturation and Nuclease Degradation,” Nature Comm. 8:15654 (2017), which is hereby incorporated by reference in its entirety). The octahedra were incubated in 55% mouse serum for 1 h and 24 h at 37° C. To allow imaging with negative stain the samples were diluted with PBS to a final mouse serum concentration of 5%, immediately before application to the negative stain grids.

The partial shells used for virus neutralization experiments in vivo were assembled at 60 mM MgCl₂ and UV cross-linked as described in (Gerling et al., “Sequence-Programmable Covalent Bonding of Designed DNA Assemblies,” Sci. Adv. 4: eaau1157 (2018), which is hereby incorporated by reference in its entirety) for 30 min using the Asahi Spectra Xenon Light source 300 W MAX-303. Three-base long, sticky overhangs were introduced at every stacking contact and one thymidine added at the ends of both oligonucleotides to covalently crosslink the triangular subunits. The sticky overhangs were necessary to compensate for the decrease in blunt-end stacking induced by addition of the thymidines for UV point welding. The shells were incubated in a 0.6:1 ratio N:P with a mixture of K₁₀ oligolysine and K₁₀-PEG_{5K} oligolysine (1:1) for 1 h at room temperature as similarly described in (Ponnuswamy et al., “Oligolysine-Based Coating Protects DNA Nanostructures from Low-Salt Denaturation and Nuclease Degradation,” Nature Comm. 8:15654 (2017), which is hereby incorporated by reference in its entirety). The DNA modified antibodies were added to the assembled shells and incubated over night at room temperature.

### Gel Electrophoresis

The size distribution of folding reactions or shell assemblies was investigated using agarose gel electrophoresis. For solutions including only shell subunits, we used 1.5% agarose gels containing 0.5×TBE Buffer (22.25 mM Tris Base, 22.25 mM Boric Acid, 0.5 mM EDTA) and 5.5 mM MgCl₂. For solutions including oligomeric assemblies such as shells, an agarose concentration of 0.5% was used. The gel electrophoresis was performed in 0.5×TBE buffers supplemented with the same MgCl₂ concentration as the solutions in which the shells were incubated in. For MgCl₂ concentration larger than 15 mM, a surrounding ice-water bath was used for cooling the gel. The gel electrophoresis was performed for 1.5 to 2 hours at 90 V bias voltage. The agarose gels were then scanned with a Typhoon FLA 9500 laser scanner (GE Healthcare) with a pixel size of 50 μm/pix.

### Negative-Staining TEM

Samples were incubated on glow-discharged collodion-supported carbon-coated Cu400 TEM grids (in-house production) for 30 to 120 s depending on structure and MgCl₂ concentration. The grids were stained with 2% aqueous uranyl formate solution containing 25 mM sodium hydroxide. Imaging was performed with magnifications between 10000× to 42000×. T=3 triangles were imaged on a Phillips CM100 equipped with a AMT 4Mpx CCD camera. All other negative staining data was acquired at a FEI Tecnai T12 microscope operated at 120 kV with a Tietz TEMCAM-F416 camera. TEM micrographs were high-pass filtered to remove long-range staining gradients and the contrast was auto-leveled (Adobe Photoshop CS6). To obtain detailed information on individual particles and investigate successful encapsulation negative stain EM tomography was used as a visualization technique. The grids were prepared as described above, and the tilt series acquired with magnifications between 15000× and 30000× using the FEI Tecnai 120. The stage was tilted from −50° to 50° and micrographs were acquired in 2° increments.

All tilt series were subsequently processed with IMOD (Kremer et al, “Computer Visualization of Three-Dimensional Image Data Using IMOD,” Journal of Structural Biology 116:71-76 (1996), which is hereby incorporated by reference in its entirety) to acquire tomograms. The micrographs were aligned to each other by calculating a cross correlation of the consecutive tilt series images. The tomogram is subsequently generated using a filtered back-projection. The Gaussian-Filter used a cutoff between 0.25 and 0.5 and a fall-off of 0.035.

### Cryo Electron Microscopy

The DNA origami concentrations used for preparing the cryo-EM grids are summarized in Table 2. Samples with concentrations higher than 100 nM were applied to glow-discharged C-flat 1.2/1.3 or 2/1 thick grids (Protochip). Samples containing shells with less than 30 nM monomer concentrations were incubated on glow-discharged grids with an ultrathin carbon film supported by a lacey carbon film on a 400-mesh copper grid (Ted Pella). The concentration of all single triangles was increased above 500 nM with PEG precipitation (Wagenbauer et al., “How we Make DNA Origami,” Chembiochem: A European Journal of Chemical Biology (2017), which is hereby incorporated by reference in its entirety). 1 ml of folding reaction (˜50 nM monomer concentration) was mixed with 1 ml of PEG, centrifuged at 21 k rcf for 25 min and re-suspended in 50 to 100 μl 1×FoB5. The DNA-origami triangles used for assembling the shells were all gel purified and concentrated with ultrafiltration as described above before increasing the MgCl₂ concentration. Plunge freezing in liquid ethane was performed with a FEI Vitrobot Mark V with a blot time of 1.5 to 2 s, a blot force of −1 and a drain time of 0 s at 22° C. and 100% humidity. The samples with less than 100 nM monomer concentrations were incubated on the support layer for 60 to 90 s before blotting. All cryo-EM images were acquired with a Cs-corrected Titan Krios G2 electron microscope (Thermo Fisher) operated at 300 kV and equipped with a Falcon III 4 k direct electron detector (Thermo Fisher). The EPU software was used for automated single particle acquisition. See Table 2 for microscope settings for all individual datasets. The defocus for all acquisitions was set to −2 μm. The image processing was done at first in RELION-2 (Kimanius et al., “Accelerated Cryo-EM Structure Determination with Parallelisation Using GPUs in RELION-2,” Elife 5 (2016), which is hereby incorporated by reference in its entirety) and then later in RELION-3 (Zivanov et al., “New Tools for Automated High-Resolution Cryo-EM Structure Determination in RELION-3,” Elife 7 (2018), which is hereby incorporated by reference in its entirety). The recorded movies were subjected to MotionCor2 (Zheng et al., “MotionCor2: Anisotropic Correction of Beam-Induced Motion for Improved Cryo-Electron Microscopy,” Nature Methods 14:331-332 (2017), which is hereby incorporated by reference in its entirety) for movie alignment and CTFFIND4.1 (Rohou et al., “CTFFIND4: Fast and Accurate Defocus Estimation from Electron Micrographs,” Journal of Structural Biology 192: 216-221 (2015), which is hereby incorporated by reference in its entirety) for CTF estimation. After reference-free 2D classification the best 2D class averages, as judged by visual inspection, were selected for further processing. A subset of these particles was used to calculate an initial model. After one to two rounds of 3D classification, the classes showing the most features or completely assembled shells were selected for 3D auto-refinement and post-processing. For the corresponding shells octahedral (O) or icosahedral (I1) symmetry was used for the last two steps. All post-processed maps were deposited in the Electron Microscopy Data Bank (EMDB) (see Table 2).

TABLE 2
Cryo-EM Imaging Conditions

| | | # of | | | Pixel | 3D map | |
|---|---|---|---|---|---|---|---|
| | Conc. | particles for | # of | Dose | size | Resolution | |
| Object | (nM) | refinement | fractions | (e/A\hat{ }2) | (A/pix) | (A) | Symmetry |
| Octa monomer | 700 | 16524 | 5 | 42.57 | 2.28 | 18.69 | C1 |
| (EMD-12009) | | | | | | | |
| T = 1 monomer | 500 | 9496 | 7 | 51.16 | 2.28 | 20.27 | C1 |
| (EMD-12010) | | | | | | | |
| T = 3 monomer | 500 | 11080 | 7 | 53.17 | 2.28 | 19.09 | C1 |
| (EMD-12011) | | | | | | | |
| T = 4_iso | 500 | 16904 | 7 | 48.53 | 2.28 | 17.22 | C1 |
| (EMD-12012) | | | | | | | |
| T = 4_equi | 500 | 34288 | 7 | 48.26 | 2.28 | 21.21 | C1 |
| (EMD-12013) | | | | | | | |
| T = 9_pent | 800 | 25053 | 8 | 47.9 | 1.79 | 14.92 | C1 |
| (EMD-12008) | | | | | | | |
| T = 9_hex1 | 800 | 38498 | 13 | 36.85 | 1.79 | 12.92 | C1 |
| (EMD-12014) | | | | | | | |
| T = 9_hex2 | 800 | 11481 | 8 | 48 | 1.79 | 15.04 | C1 |
| (EMD-12015) | | | | | | | |
| Octa shell | 130 | 3384 | 11 | 42.71 | 2.28 | 19.64 | O |
| (17.5 mM) | | | | | | | |
| (EMD-12016) | | | | | | | |
| T = 1 shell | 110 | 2578 | 10 | 51.11 | 2.28 | 21 | I1 |
| (20 mM) | | | | | | | |
| (EMD-12021) | | | | | | | |
| T = 1 shell | 50 (lacey | 720 | 7 | 31.26 | 2.28 | 22.21 | I1 |
| (25 mM) | carbon grid) | | | | | | |
| (EMD-12024) | | | | | | | |
| T = 3 shell | 20 | 612 | — | 22.96 | 3.71 | 36.15 | I1 |
| (20 mM) | | | | | | | |
| (EMD-12019) | | | | | | | |
| T = 4 shell | 21 (T_iso) | 255 | — | 25 | 3.71 | 47.87 | I1 |
| (25 mM) | 7 (T_equi) | | | | | | |
| (EMD-12020) | | | | | | | |
| spiky shell | 150 | 3847 | 8 (dataset1) | 25.76 (dataset1) | 3.76 | 22 | I1 |
| (22.5 mM) | | | | | | | |
| (EMD-12049) | | | 11 (dataset2) | 30.00 (dataset 2) | | | |
| triangular brick | 1000 | 38132 | 7 | 78.6 | 2.28 | 11.9 | C1 |
| (EMD-12046) | | | | | | | |
| Octa_half | 180 | 6801 | 10 | 40.44 | 2.9 | 20.41 | C4 |
| Shell (30 mM) | | | | | | | |
| (EMD-12007) | | | | | | | |
| Octa_half | 40 | 2707 | 7 | 44.38 | 2.9 | 23 | C1 |
| shell (30 mM) + | | | | | | | |
| HBV core | | | | | | | |
| (EMD-12044) | | | | | | | |
| T = 1_half shell | 180 | 8725 | 10 | 40.44 | 2.9 | 15.16 | C1 |
| (2 triangles, | | | | | | | |
| 30 mM) | | | | | | | |
| (EMD-12045) | | | | | | | |
| T = 1_half shell + | 50 | 1770 | 7 | 44.79 | 2.9 | 23 | C5 |
| HBV core | | | | | | | |
| (2 triangles, | | | | | | | |
| 30 mM) | | | | | | | |
| (EMD-12022) | | | | | | | |
| T = 1_15 mer shell | 210 | 3194 | 10 | 29.17 | 2.9 | 22.3 | C5 |
| (3 triangles, | | | | | | | |
| 30 mM) | | | | | | | |
| (EMD-12023) | | | | | | | |

In vitro Virus Blocking ELISA

Various concentrations of assembled half-T1 shells were incubated overnight at room temperature with 2 nM oligonucleotide-conjugated capture antibody (anti-HBc 17H7, Isotype IgG-2b) in FoB30-T (FoB30+0.05% TWEEN-20). The next day the pre-incubated mixtures were added to 5 pM HBV core particles and incubated overnight at room temperature, yielding 1 nM capture antibody, 2.5 pM HBV core particle and 0-200 pM half-T=1 shells. A flat-bottom transparent 96 well microplate (Nunc MaxiSorp) was treated overnight at 4° C. with 100 μl/well anti-CAgHB antibody (1 μg/ml in PBS). After washing 4 times with 200 μl/well PBS-T (PBS+0.05% Tween-20) the well surface was blocked by incubating with 200 μl/well 5% bovine serum albumin in PBS for 2 hours at room temperature. After washing 4 times with 200 μl/well FoB30-T, 90 μl of the pre-incubated samples were added to the wells and incubated for 2 hours at room temperature, followed by washing and subsequent incubation for 1 hour with 100 μl/well horseradish peroxidase conjugated detection antibody (anti-CAgHB-HRP in FoB30-T). After washing with FoB30-T, 100 μl/well HRP substrate (3,3′,5,5′-Tetramethylbenzidine, lifetechnologies) was added and product formation was monitored in time by measuring the absorbance at 650 nm with a 60 s interval in a platereader pre-equilibrated to 30° C. (CLARIOstar, BMG labtech). HRP activity was calculated by fitting linear regression slopes to the linear regime of the kinetic data (typically the first 5 minutes). Virus blocking efficiency was calculated relative to a control of HBV core particles only and blank measurements where no HBV core particle was present during all the incubation and washing steps. All experiments were performed in triplicates. Antibodies used for the ELISA were kindly provided by Centro De Ingenieria Genetica y Biotecnologia de sancti spiritus in Cuba.

### Helium Ion Microscopy (HIM)

Imaging was performed with negative-stained TEM grids coated with a 5 nm layer of AuPd using a Quorum Q150 T sputter coater in ORION Nanofab (Zeiss). An acceleration voltage of 30 kV and a beam current of 0.3 to 0.4 pA were used. The images were acquired in scanning mode with an Everhart-Thornley 2 k detector.

### Production of HBV Core Particles

Hepatitis B virus core particles of genotype D (subtype ayw2) were produced recombinantly in E. coli K802 and BL21 cells (purchased from purchased from the Latvian Biomedical Research and Study Centre, Riga, Latvia). Briefly, particles were obtained by sonication and clarification from bacterial protein extracts and purified by ammonium sulphate precipitation and subsequent anion exchange and size exclusion chromatography as described (Sominskaya et al., “A VLP Library of C-Terminally Truncated Hepatitis B Core Proteins: Correlation of RNA Encapsidation with a Th1/Th2 Switch in the Immune Responses of Mice,” PloS one 8: e75938 (2013), which is hereby incorporated by reference in its entirety). Final preparations were constantly kept at 4° C. in the dark in conventional PBS (including 0.05% NaN₃, 1 mM DTT).

### Production of Anti-HBC Antibody

Anti-HBV core (anti-HBC) antibody 17H7 (Isotype IgG-2b) was produced by the Monoclonal Antibody Core Facility at Helmholtz Zentrum München in Munich (HMGU). Briefly, mouse HBc-recognizing B cells were generated by common hybridoma technology. The mice were challenged with the peptide NLEDPASRDLVVC (aa 75-86 of HBV core). Mouse hybridoma clones were selected and secreted antibodies were analyzed by immune staining and precipitation of HBcAg and ELISA for native antigen recognition and by Western Blot analysis for detection of denatured antigen. Final 17H7 preparations were purified via standard affinity chromatography using a protein A/G column and concentrated to 0.8 mg/mL (5.33 μM) of protein and kept in conventional PBS (137 mM NaCl, 10 mM Phosphate, 2.7 mM KCl, pH 7.4) at 4° C. in the dark.

### Cell Culture and Neutralization Assays

HEK293T (human embryonic kidney cell line, DSMZ) cells were cultured in Dulbecco's Modified Eagle's Medium (DMEM, Gibco, cat. no. 31966047) with 10% heat-inactivated Fetal Bovine Serum (FBS, Sigma-Aldrich, cat. no. F9665). Cells were cultured routinely in a humidified incubator at 37° C. with 5% CO₂. AAV2 carrying eGFP (Biocat, cat. no. AA002-GVO-GC) were utilized for transduction experiments, where the concentration of infectious particles was determined by titration as per the manufactures protocol. Briefly, cells were seeded in 24-well plates at 80,000 cells mL⁻¹ 16-24 h prior to transduction, and harvested 72 h after transduction for quantification of transduction efficiency by flow cytometry. Samples were acquired and analyzed using Attune™ NxT Flow Cytometer and software (Thermofisher) respectively. 20,000 single cell events, gated on side scatter area vs height were recorded for analysis. eGFP was excited by 488 nm laser, and emission was measured with a 530/30 nm bandpass filter. Untreated cells were used as a negative control. Concentration of infectious particles was determined to be 1.23×10⁹ IFU mL⁻¹. Total number of AAV2 virus particles was measured by ELISA as per manufacture's protocol (Progen, cat. No. PRATV), and determined to be 2.24×10¹² VP mL⁻¹.

For neutralization experiments, cells were cultured as above. 48-well plates were coated with poly-L-lysine (Sigma Aldrich cat. no. P2636, 0.1 mg mL⁻¹, 10 min r.t. incubation) and then washed 2× with H₂O and then PBS. HEK293T cells were seeded at 80,000 cells mL⁻¹ 16-24 h prior to transduction. Stock solutions were prepared for the overnight binding of conjugated anti-AAV2 to half shell origamis. Binding occurred in the presence of 0.1 mg mL⁻¹ bovine serum albumin (BSA). Similarly, conjugated anti-AAV2, and half shells without antibody were also prepared in an identical manner.

The next day, the half shells were coated with PEG-oligolysine/oligolysine by r.t. incubation for 2 h. Next, each of the different titration conditions were prepared and diluted to a total of 33.5 μL per condition with PBS. 4 μL of diluted AAV-2 sample ( 1/100, in PBS) was added and mixed, and samples were left to incubate (2 h, r.t.). Cells were washed with PBS and 62.5 μL of DMEM with 2% FBS was added to each well. Mixtures (37.5 μL) were then added dropwise to each well. Cells were incubated for 2 h, before 100 μL of DMEM with 18% FBS and 1× antibiotic/antimycotic was added. The cells were incubated for a further 22 h before media was removed, cells were washed with 1×PBS, and 250 μL of DMEM with 10% FBS and 1× antibiotic/antimycotic was added. At 48 h post-transduction, the cells were trypsinized and prepared for flow cytometry. Transduction efficiency was quantified by flow cytometry as above. Statistical analyses were performed with Graphpad Prism (GraphPad Software Inc.).

For epifluorescence imaging, the procedure was identical as above, with the exception that the cells were seeded in 8-chambered well slides (Nunc™ Lab-Tek™, Thermofisher). After the total 48 h time point, cells were washed with 1×PBS and then fixed with 2% paraformaldehyde (PFA). Cells were washed again (1×PBS), and cell nuclei were stained (Hoescht 3342, diluted in PBS, 5 min, r.t.). Cells were washed with PBS, and the samples were mounted using Fluoromount-G aqueous mounting media. Samples were imaged using a Tikon Eclipse Ti2-E inverted microscope, using a 10× objective. Images were collected using NIS-Elements AR software, and processed using ImageJ.

### Viability Assay

Cytotoxicity was quantified by cell viability following 24 or 48 h incubation of cells with the half-shell mixtures. HEK293T cells were seeded in poly-L-lysine treated 96-well plates at 80,000 cells mL⁻¹. Cells were allowed to settle overnight, media was removed and cells exposed to half shell mixtures in an identical procedure to the neutralisation assays. Cells were incubated with half shell solutions for a further 24 h or 48 h, and alamarBlue reagent (Invitrogen, 10 μL per well) was added. Plates were mixed and incubated for 4 h before being read on plate reader (CLARIOstar). Absorbance readings at 570 nm and 600 nm were taken, as per manufacturer's protocol. Measured values were normalized to control wells, which were treated identically, but received PBS containing no origami structures. All conditions were measured at least in triplicate.

### DNA Sequences

Staple sequences used in the construction of icosahedral shells formed by triangular subunits, as described herein, are set forth in the following tables where * denotes Cy5 at the 5′ end and † denotes Cy3 at 5′ end.

TABLE 3
T-octa

| | | SEQ ID |
|---|---|---|
| Name | Sequence | NO: |
| core_1 | TTTGGGAATATTCACAAACACAAAGTTACAAAGACAGAAGCGCA | 4 |
| core_2 | CAGGCAAGTTTCATTCCATATAACGTACGGTGAACAACCCTCAACATT | 5 |
| core_3 | TGTCGTCTTTCCAGACGTTAGTAAAACGATTGGCCTTGATT | 6 |
| core_4 | GTCGGATTATAACCTATGTTTACCAGTCCCGGAATTTG | 7 |
| core_5 | TTAAACGGACCTAAAACGAAAGGAACGAGGGGGAGTGTACTGGTAATA | 8 |
| core_6 | GCAGAACCACCACCAGAGCCGCCTTTCAACAACTAAAGG | 9 |
| core_7 | ATCGCACTAGCGAGTTCTGGAAGATCAACGTAACAAAGC | 10 |
| core_8 | AGAGGACAGGAACCGACCCAGCGAACACTAAAAACGAGGGTAGCA | 11 |
| core_9 | TGAATTTTCCACCCTCCATCGCCCCAAAAG | 12 |
| core_10 | CACTACGATTACCCAAAGTAAGCGTCATACATGAATTT | 13 |
| core_11 | TTTTTCACCTGTATGGGATTTTGCTAAACAACGCCAGCATTGGAAAGC | 14 |
| core_12 | CGGCCAGAGCGCCTGTGCACTCTGATCAGATG | 15 |
| core_13 | AGGCACCAGTAAAATACTTTGAGGTGCAGGGAGTTAAAGGCGAAACGT | 16 |
| core_14 | TTAGGATTAGCGGCGCAGACGGTCAAGTAACAGCGCATAGG | 17 |
| core_15 | AAGGAACAGTTTCAGCGGAGTGACCAGCCAG | 18 |
| core_16 | GAGATGGTTTAATTTCGGTCAGGAATAATGCTTAGTTTGACCATTAGA | 19 |
| core_17 | CCGATATATTCGGTCGAATTGCGAAGTTTCCAGATGATACAGGAGAGA | 20 |
| core_18 | GGAATTAAGTTTAAAGAAACGCCAGAAGGAAACCGAGACCGGAACAGAGCCAG | 21 |
| core_19 | GGTTTCTGCCAGCACGCGTGCCTGGGAGGTTTAAACAGTCCTTTACA | 22 |
| core_20 | CAGTGAATTAAATATGCAACTAAAAGTTGATTTATCGGCC | 23 |
| core_21 | GAAAAGGTAAAACATTATGACCCTGTAATACTTTTGCGGG | 24 |
| core_22 | TTTAACGTCAAAATACGAACCTCCCGACTTGCGTTCTTCGCGTCCGT | 25 |
| core_23 | CTGAGGCTACTAAAGACTTTTTCAGCCGAGGCAAAAGAATTTATACCA | 26 |
| core_24 | CACGACAAGAACCGGATATGTCAGTGCCTTGATCATAAGGATGAACG | 27 |
| core_25 | CGCAGTGTATTTTTGTATTTTGTTAAAATTACTTTGAACAAGAGT | 28 |
| core_26 | AACGGAACCTCCGGCCAGAGCACCTTTGACCACTGACCA | 29 |
| core_27 | CTCCATGTTACTTATGAAAAAAAAAGGCTCACGCATAA | 30 |
| core_28 | GCATCAGACGATCCAGTGCTCATTAGCTTTCA | 31 |
| core_29 | CACCGCTTCTGGTGCCGGAAACCAATAGTAGTTGCGAACGAGTAGATT | 32 |
| core_30 | GAGGGGACGAACAAACACATGTTTAAGGCTTGCCCTGACGAGAAA | 33 |
| core_31 | AAATCACCGGAACCAGAGCCACCGAAACGCAATAATAA | 34 |
| core_32 | CCGCCACGGGAACGGCTCCGTGGGACGACAGCCCAATTC | 35 |
| core_33 | CACCGGAAACAATCGGATCCTCATAAATGTGCGCATTAA | 36 |
| core_34 | CCGCTTTTCGTAATGCGAATAGAACCGTTCC | 37 |
| core_35 | CAGCGAAAGACAGCATCGGACACTCAT | 38 |
| core_36 | ACGGCTACAGAGGGGGGGATCGTCACCCTCAG | 39 |
| core_37 | CGCCTCCCTCAGAGCCGCCACCCTTAGCCGAAAATAAATC | 40 |
| core_38 | TTTTGCTCGTTGAAAATCTCCGGAATAATAATAGCCAGAATGACAGGA | 41 |
| core_39 | TTAAGCAATAAAGCATCCAATAAATCATACTTTCCGGTCAGGAAGACAGCGCC | 42 |
| core_40 | CCACCCTCAGAGCCACCACCCTCGCTAATATGCAGTCTCTGGCTTTT | 43 |
| core_41 | CTGTAAATACCAAGTTTTAGTTAATAACCT | 44 |
| core_42 | TCTGACCTAAATTTAAAGAGTCAAAATTACTATAAAAGTTTGAGTGAA | 45 |
| core_43 | TGGTTTGATAAGAATAAACACCGGCAACCAACGCTCAACAATTTAGGC | 46 |
| core_44 | TAAATAAGTATCATATTAGCTTAATTAAATC | 47 |
| core_45 | TGTGTGAAATTGTTATCATCGAGAGGGTTGAT | 48 |
| core_46 | ACAAGCAAACCAGCGCCAAAGACAAGGTAAATCCGTAACA | 49 |
| core_47 | ACCTTGCTGAACCTCAAATATCAATTTAGACATTGAGGAT | 50 |
| core_48 | AGCCAGCAGCAAATGAAAAATCTAAGTGTTTTTATAAT | 51 |
| core_49 | TCAGCTTGCTTTCGAGCCTGATAAAGCCCGGAGTATTAA | 52 |
| core_50 | AGCCTTTAATTGTATGAGGTTTACCCATGTAATTGACGG | 53 |
| core_51 | ACGACGACAATAAAAATGAGACTACCTTTTTTTCATCT | 54 |
| core_52 | ATTGTGTCAGATTTGTATCATCGACAACGCCAATAGATA | 55 |
| core_53 | ACAAGAAATTATCAACAACATGTAGTAGGGCTCATATTTA | 56 |
| core_54 | CTACCATATCAAGCTAATGCAGAACGGAGCGGACGAGCATG | 57 |
| core_55 | AATTCCTTATCATTCCAAGAAACCACCAGAAGCGCCTGTAATAATAT | 58 |
| core_56 | AATGGAATTGAGGAAGGTTATCTTAGAATCCCGCTGAGA | 59 |
| core_57 | CAAATTCTCTGTTTAGGCGTTAAAAATACCGACCGTGTGAGTGAATTT | 60 |
| core_58 | GCGTTATAGGGTATTACTTTGCCCGAACGTTAACTCGT | 61 |
| core_59 | GAAAAAGCTACCAGTATAAAGCATGTTCAAATAAGAACATTATCATTT | 62 |
| core_60 | AAAGTGTAACACAACATACGAGCCCGGGTACC | 63 |
| core_61 | TTATCAAACGTCGCTATTAATTAATTTTCCCTAAAATATCTTACAAAC | 64 |
| core_62 | GATTAAGATTGAAAACATAGCGAGTACAAAC | 65 |
| core_63 | CGTCACCAAATAGGTACATTCAAAACCAAGTACCGCACT | 66 |
| core_64 | AATAGGAAGTACCGCCTATGGTTTGCCGTTTTTATTTTCATCGTA | 67 |
| core_65 | TATATGTGAGTGAATAACCTTGCTTATTAGAGCCGTCAGAG | 68 |
| core_66 | ACATTAATTGCGTTGCGCTCACTTTGGAACACATATTCCTGGCGAGA | 69 |
| core_67 | CTGTAGCATTCCACAGACAGCCCTATGAAACCAAATTATTCATTAAAG | 70 |
| core_68 | GTACTCAGCGGTTTAGCTTGATACCGATAGTTGCGCCG | 71 |
| core_69 | AGCGACAGCCGATTGAGGGAGGGAAAAGGGGGGTATCACCATAAGTAT | 72 |
| core_70 | TTAGCAAGCCCTTATTAGCGTTTGCCATCTTTTCATAATC | 73 |
| core_71 | GCCAACAATAGATAATACATGGAACGGTCAAACTATGAGCGGGC | 74 |
| core_72 | CAAATCAGATATAGAACAAACGTAATCAATAGGTGAATTATCACCGTC | 75 |
| core_73 | AGCAGCTGGTAATGAAGAACTACGCCAGAATCCTGAGAAAGCATCATAGAACC | 76 |
| core_74 | CCGCTCACGAGGCTGATTCTGAAACATGAAAGTCCTGAAATCGGC | 77 |
| core_75 | AGCTTGACGGGGAAAATGAGTGTTGTTCCAGTGCCCGCTTTCCAGTC | 78 |
| core_76 | TATTTGCAATCATAGGTCTGACATTAGTGAATATTAGACTTTTAGGAG | 79 |
| core_77 | GCGTCAGACTGTACAGCACCGTAATCAGTTACAACGCCTGAGTTTCTTAAACA | 80 |
| core_78 | TCAATATCTGGTCAGTTGGCAAACGCGCTTAAATTCGACATTAATTT | 81 |
| core_79 | TATGAGCCTCAATCCGCCGGGCGCTAATGAAT | 82 |
| core_80 | TAACCCTCGTTTACGAAACTGGCTCATTATACACATCCCTTACACTG | 83 |
| core_81 | TTGGGTAAGATGTGCTGCAAGGCCAAAATTA | 84 |
| core_82 | ATAGCTCTCACGGAAATTTTCCCAGGTTGTGTAATCGTAAAACTAAGC | 85 |
| core_83 | CGCACAGGATAAAAAATTTCTGCTCAGAAACAGCGGATCAATGCAAAT | 86 |
| core_84 | ACCTGAGCGAGAAACATTGCAACACGGTCCACGCTGGTTTGCCCC | 87 |
| core_85 | ACATCGACCGGCCTTTAGTGAGGTCTGGTCGCAATGCATGTCAATCAT | 88 |
| core_86 | GAAGCCTTTATTTCAACGCAAGGCCGAAAGACTTCAAA | 89 |
| core_87 | AAGAGACGCATTTGCCGCCAGCAGGCTTGAAGGGTAAAGTGGTCCGTT | 90 |
| core_88 | AACGTCAGCGTGGTTTGCCAAGCTTTCAGAGTGAAGGG | 91 |
| core_89 | GTGGCACATATTAACACCGCCTGCAACAGTGCCACGCTGA | 92 |
| core_90 | GTGCGGGCCGTTGGTGTCTCCGTGGGTGGA | 93 |
| core_91 | AAAGGTAATAAGAGAATATAAAGCCGTTCCGGCACCGTC | 94 |
| core_92 | GTTCCGAAATCGGCAAAATACTTCATATTACCCACACGACCAGTAATA | 95 |
| core_93 | TAACAATTTCATTTGAATTACCTTGGCTATTATGGCAGATTCACCAGT | 96 |
| core_94 | GCGCCAGGGTGGTTTTAGAGAGTTGATTTTCA | 97 |
| core_95 | TCTTTTCACTTCTGATAATCCTGATTGTTTGGTGGTGCGCAGGCGC | 98 |
| core_96 | CATCCCACCAACGGCAGCAAACGCTAAACGATGCTGATTG | 99 |
| core_97 | ACGGGCAACAGTGATAATCAGAAAAGAATGCGCAACCAG | 100 |
| core_98 | GTACCTTTTATATAAAAGACAAAGAACGCGAGAAAACT | 101 |
| core_99 | CAAGAAAAGAATATAACATTTTGTCACCGCCTGGCCCTG | 102 |
| core_100 | AAAAAAGCATTGCCCTAAACAAGAGAATCGACTGAGAG | 103 |
| core_101 | AACCAATAGGAACAGCAGCAACCGCAAGCCCCATGTCCAGC | 104 |
| core_102 | GGTCAATGCCGGAGAGGGTAGCATCAAATTTTTGCGAGAAGTTT | 105 |
| core_103 | TTTTAGAACCCTCATATATTTTAAAGCGGATTGCTATTTT | 106 |
| core_104 | GCAGCAAGGGAAAAACGCTCATGGAATGGATTATGATGAA | 107 |
| core_105 | CCTAAAACACGCTCAATCGTCTGAAAATACCTCAGTAACAGGTTTAAC | 108 |
| core_106 | CACCAAGAGCTTAAGAGGTCAAAGATTAAGAGGAAGCATAAAAATCTGTTTAG | 109 |
| core_107 | CTATATGTAAATGCTGTACCGACAGTCAGATGGATTATA | 110 |
| core_108 | GCGCAACTGTTGGGAAGGGCGATCGCTGATAAATTAATAAC | 111 |
| core_109 | ATCGAAAGGCCGGAGACAGTCAAGAAAGGGGCGCCAGGG | 112 |
| core_110 | GTTGTAAACTCTTCGCTATTACGCCAGCTGGCATCACCATGCTATCAG | 113 |
| core_111 | AACTTAAAATCCCGTAGATTAAGTCTGGAGC | 114 |
| core_112 | AAAGGTTTCTTTGCTCGTCATAACAGTCAGGAAGATTGTAAAACCAA | 115 |
| core_113 | CGGCTTAGGTTGGGTTACATCGGAAAAGAAGATTTACAT | 116 |
| core_114 | CATCAAAAACGACGGCCAGTGGGCGTCACGACCTACAAAGCAATATGA | 117 |
| core_115 | CGAACGAACCACCTGCGCGAACTGATAGCATTACATTACAAACATCCAATCGC | 118 |
| core_116 | CCTGAGTAATGTGTAGGTAAAGAGAATCGTCGTCATTGCTGAACGGT | 119 |
| core_117 | GTGTACAGACCCTATTTTTTTTGGAACCTATTAGACTCCTCAAGAGAAGG | 120 |
| core_118 | GCCGGAAACGTCACCACATAGTTAGCAAGCCCACAATGATTTTTAACAAC | 121 |
| core_119 | CAAAATCACCAGTTTTTAGCACCATTACCAACCGATTTTTTTGAGCCA | 122 |
| core_120 | AGCGCGAAACAAAGTTTTTACAACGGGAAATCCGCGACCT | 123 |
| core_121 | CGCGAGGCTTTTTTTTTAGCCGCGCCCAATAGCAAGACATATAA | 124 |
| core_122 | AAAATTCAACCCTCAGAATTTTTCGCCACCCTCCACGGAA | 125 |
| core_123 | AGAGCCACCACCCTCATTTTTTTTCAGGGATAGCGTAACGATCTAAAGTT | 126 |
| core_124 | GTTTCCGAGCTTTTTTGAATTAATGCCCCCTGCAGGTGCCCGTA | 127 |
| core_125 | GGAATACCCAAAAGAAATACATAATTTTTGGTGGCA | 128 |
| core_126 | TTTTTTTTTTCACAGAAAATACCTGGCATGATTAAGACTCATAGCC | 129 |
| core_127 | AAAACAGGCAGAACCGAGTACCAGGTTTTTGGATAAGTGCCGTCGAGA | 130 |
| core_128 | CATATGAAAATAGCAGTCGTAATCATGGTTTTTCATAGCT | 131 |
| core_129 | AGCGAAAGCTTTGAATCGTAAAACATTTTTAAATAAAGAAATTGCGTA | 132 |
| core_130 | CCCATCCTAATATGGCATTTTTTTCATCAATAATAATGGAAGGGTTAGAA | 133 |
| core_131 | ACAAAATCGCGCAGAGGTTTTTGAATTATTCAGAAACAGTACATAAATCA | 134 |
| core_132 | CTTCTGACCTGATTTTTAGCGTAAGAATACAAAGGTTTTTACATTCTG | 135 |
| core_133 | GACAATATTTTTGAATTTTTAATGTTTCAATTTTTTCAATTTTTTATATT | 136 |
| core_134 | AGAGGCATTTTCGATTTTTCCAGTAAAGTAATTCTGTCCA | 137 |
| core_135 | GCCAGCCAATAACGGATTTTTTTGCCTGATTGCGGCCTTG | 138 |
| core_136 | ATCCTTTTTGAACATTTGATTAAAAAGAGTCTGTCCATGCGGTCAG | 139 |
| core_137 | TATTGGCGGCCTTTTTACGCTGATTATCAGATGTTAATTATCAT | 140 |
| core_138 | GCGAAGCCGGCGAACGGGGGGGAGAGGCTTTTTGTTTGCG | 141 |
| core_139 | AGTGAGGCCACCGAGTGTAATAACTTTTTTCACTTG | 142 |
| core_140 | TAGCCCGATTTTTATAGGGTATCCTGTTTGATGGTGCCTGAGTA | 143 |
| core_141 | GAACCAGACGACGATATGCAGCCAGCGGTTTTTGCCGGTG | 144 |
| core_142 | CTATATTTTCATTTTTTTGGGGCGCGAGCTTACATTTTTTTCGCAAAT | 145 |
| core_143 | TTTTCGTCTCGTCGTTTTTTGGCAGCGTGCCGGACTTGTA | 146 |
| core_144 | CTTATGCGTTTTTTTTTAAGGTAGTAAATTGGGCTTTTTTGATA | 147 |
| core_145 | GGCATCAATTCTACTAGGCAAAGCGCCAGTTTTGAGAGATTTTTAGACTT | 148 |
| core_146 | TAGATGGGCGCATCGTATTTTTCCGTGCATCTGCCATTCGCCATTCAGGC | 149 |
| core_147 | GTAGCTCAGGCGGATTGATTTTTCGTAATGGGGATGGCTT | 150 |
| core_148 | ATTGTTTTTTGAATTTAGAGAGAGCTTCAAAGCGAACCTTGTACCA | 151 |
| core_149 | CCCCCTCCGGGTTTTTTACCATAAGCAAATATTAGGAAAACAGG | 152 |
| core_150 | ATCGCGTTTTAATTCGTACCTTTATTTTTTTGCTCC | 153 |
| core_151 | TTTGCAAAATAGGTCAATAATTCGCTTTTTTCTGGCCTTCCTGTAGCC | 154 |
| core_152 | CTTACGGCTGGTAAATTTTTTTTAAACGTTAATTAAATCAGCTCATTTTT | 155 |
| side1_recess_1 | TTGTGAATTACTGTGTTCAGCAAATCGTTAACGGCTGGTGCTG | 156 |
| side1_recess_2 | AGCATTAACCTCAGAGCATAAAGCTAAATCGGAGACCGGAAGC | 157 |
| side1_recess_3 | AATCTTTGCATGCGGGGGGCCGTTTTCACGGTCCAAAATAAACA | 158 |
| side1_recess_4 | GCCATATTATTCAAAGTCAGAG | 159 |
| side1_recess_5 | AAACTCCAACAAACTTTAATCA | 160 |
| side1_recess_6 | GGTAATTGAGCAGAGCCGCCACCAAAGAATTAGCAAAA | 161 |
| side1_protrusion_1 | TGAATCTTACCAAGCCAGTTAATACCGGGCTGGCTGACCTTCAT | 162 |
| side1_protrusion_2 | CCTTTTTAAACTGAACTTAAGCCCCACCCAGCTACAATTTTATCC | 163 |
| side1_protrusion_3 | GAGAATTAGAAAAGGCTATCTTACCGAAGCCAGAGAGATAACC | 164 |
| side1_protrusion_4 | TGAAATAGCAATATAAGCAGACAGAACCGGGTTGAGGCAGGTCAG | 165 |
| side1_protrusion_5 | TCAAGATTAGTTGATTTTTTGTTAGACGGATAACATACTCATTAA | 166 |
| side1_protrusion_6 | CACAAGAATTGAGACCCTGAATATCCCAAAGTTTTAACGGGTCA | 167 |
| side1_protrusion_7 | TCCAAATCTAATTTCGCTAACGAGCGTCTTTTGAAGCCTTAAA | 168 |
| side1_protrusion_8 | TCCAGAGCAAGAAACGCTATTTTGAATAATAAGAGCAAGAAACAA | 169 |
| side2_recess_1 | TATTCTAAGAAAGCCTCCTCACAGTTGAGGATCCCGGAAGCAT | 170 |
| side2_recess_2 | TAAAGCACTAATGCGCGTAACC | 171 |
| side2_recess_3 | CAGTATGTTAGGGCTTATCCGG | 172 |
| side2_recess_4 | ACCACACCCGCTCAACAGTTGAAACAAGTTTGCCTTTA | 173 |
| side2_recess_5 | TGTCTTTCCAAGCCTGGGGTGCCTAATGAGTGATCGAGGTGCCG | 174 |
| side2_recess_6 | ATCGATAGGCGCGTTTTCATCGGCATTTTCGGTCCTTATTACG | 175 |
| side2_protrusion_1 | AAAGAACGTGGACCGATTTAGGCTAGGGCGGGAAGAATTAGAAGT | 176 |
| side2_protrusion_2 | AACAGGAGAGTGTAGCGGTTGCTTCAAAGGGCGAAAAACCGTCTA | 177 |
| side2_protrusion_3 | GCTGGCAGCCGATTATCAGAGCGGGAGCTAATGCGCCGCTACA | 178 |
| side2_protrusion_4 | CACCCAAAGGAGCCCCTCCAACGTTGACGAGCACGTATAACGTGC | 179 |
| side2_protrusion_5 | TCAGGGCGATGGCTTTTGGGGGCTAACTCTAGAAACCAATCAAT | 180 |
| side2_protrusion_6 | GGGCGCGTACTATGGTCACGCATCGGAACTGCGGAACAAAGAAC | 181 |
| side2_protrusion_7 | TTTCCTCGTTAGAAAAGGGATACCCTCAACACTAACAACTAATAG | 182 |
| side2_protrusion_8 | CCTAAAGTCAAGTTCCACTACGTGAACCATAGAGTCCACTATT | 183 |
| side3_recess_1 | CCAATACTGCGTTCAAAAGGGTGAGCCATTAAAAATAC | 184 |
| side3_recess_2 | CGCCAAAAGGACTGGATAGCGT | 185 |
| side3_recess_3 | CCAGTGAGTTTCGCACGGGTCACTGTTGCCCTGCGGCTGGCAGATACATAA | 186 |
| side3_recess_4 | GTCTTTAAAGCAGAAGATAAAACAGAGGTGAGCACGCAAATTA | 187 |
| side3_recess_5 | AATCAAAAGAAGGAAACCTGTCGTGCCAGCTGCATGGTTGCGG | 188 |
| side3_recess_6 | ACCGTTGTAGCAATCCCTTATA | 189 |
| side3_protrusion_1 | CTGACTATTAGTAAAAATGCTTTAACTAACGGAACAACATTATTA | 190 |
| side3_protrusion_2 | GAATCCCCCTCAATGTTTAGAATTACGAGATGTACCCCGGTCTG | 191 |
| side3_protrusion_3 | CAGGTAGAAAGATAACTAATGTAATGGGTATCAGCGGGGTCATT | 192 |
| side3_protrusion_4 | GGGGTAATATAGTCAAATCAGGTCTTTACCATAAATATTCATT | 193 |
| side3_protrusion_5 | GCATAGTCACATTCTCATCAGTTGAGATTTACGTTGGGAAGAA | 194 |
| side3_protrusion_6 | AAATCTACGTTAACACTATCATGCCAGAGGAGAGGCTTGAGAGAT | 195 |
| side3_protrusion_7 | AGGAATACAAGAGCAATAAAACGAAACAGTTCAGAAAACGAGAAT | 196 |
| side3_protrusion_8 | GACCATAAATCAAAGAAGCAAATGCAATGTATTCAACCGTTCTAG | 197 |

TABLE 4
T = 1

| | | SEQ ID |
|---|---|---|
| Name | Sequence | NO: |
| core_1 | TGATATAAGTATAGCCAACCAATACAAAGAATTAATTAATATTTTGT | 198 |
| core_2 | GGAAACCACGGTGCGGGCCTCTTCAGCCCAATGTATAAGCAAAAGCCC | 199 |
| core_3 | GCTATTACTAAAATTCGCATTGCTTTAAACAGTT | 200 |
| core_4 | CCATGTTTACATAGCTATCTTACAGGAAACAATG | 20 |
| core_5 | GCCACCGCCACCCTCAGAGAGCCCAATAATACGAGGAAAGTGTATCA | 202 |
| core_6 | CGCCACCCTCAGAACCGGAATAGCGCAATAATAGGACTGTAGCGCGT | 203 |
| core_7 | AATACCCAACGGAATACAAGATAGCAGCACCG | 204 |
| core_8 | AGTGTACCCCGGTTGCTGCTCCAATTTTCAGGGATAGCA | 205 |
| core_9 | CTTATTACGTGGCAACATATAAAACATATGGTCAGAGAGACGAGCGTC | 206 |
| core_10 | TGTTACTTAGCCGCATTAGACGGAAGACACCAAAGAACT | 207 |
| core_11 | TTAATGCCAGAATCGAGAATATAAACGGTGTACCAACTTTGAAAGAGG | 208 |
| core_12 | AAAACGACGGCCAGTGAATCAATAAAATAGCACTAATATC | 209 |
| core_13 | GCAGACGGTCAATCATTCCGCGACATAATCAGAAATATTTCTCAGAG | 210 |
| core_14 | AAAATAGCAAAGTCAGAGGAGAAAACTTTTTC | 211 |
| core_15 | AGCCCGAGGAAGATTAGGAACCCATAAATTGTGTCGAAAAAGGGAAC | 212 |
| core_16 | GGCGATTATGGCGAAAGGGGGATGAATAAATTTTTGTTAAATCAAAA | 213 |
| core_17 | GCCGAACAAAGTTACCAGTAAGACTCAGAGAGATACTGAACA | 214 |
| core_18 | GCCTTTAGCGTCACAAAGGATAAAAATTTTTAGAACCCT | 215 |
| core_19 | TCAAGTTTGGCATGATAAGGAAACAGAGCAAGTTTAGTACACGTTGT | 216 |
| core_20 | AATAAAGCAAATTGTAAACGCGGCCACCCTC | 217 |
| core_21 | CAAAATCAAACGTCACAGAAAATTGAAACGCAGAGAATTAAACCCACA | 218 |
| core_22 | TTTTGTCAGGATATCAGGTCATTGCCTGAGAACCCAGCT | 219 |
| core_23 | CCAGTAGCACCGTCACCGACTTGAAACGCTAAATAACATA | 220 |
| core_24 | TGGGTTATATGGAAACAGTACATACCAAGCTTTCAGAGGTGGAGCCG | 221 |
| core_25 | TGTCAATCATATTTAAACGAGGCACAATTTTTTAGTTGC | 222 |
| core_26 | AGAACGCGAGGCGTTTTAGCGAACGGCAGAGG | 223 |
| core_27 | AAACATTCAAAAACATGAACGGTCGAACTGACAGACCAG | 224 |
| core_28 | GTCTGGAGAACTAGCAGACAGTCATGTAATACTTTTGAGG | 225 |
| core_29 | AATCGTAACAAACAAGGGAGAGGGTAGCTATTTTTGAGAAGGCCGGA | 226 |
| core_30 | CTTGCGGGAGGTTTTGAAGCCTTAAAAAGCCTAAAACAGGGAAGCGG | 227 |
| core_31 | TTTAATGGAGCCAGTAATAAGAGAGTATTCTAAACAGCCATA | 228 |
| core_32 | TATTTTGCACCTTCATCAAGAGTAGCGCATAGGCTGGCTG | 229 |
| core_33 | CAATGAAACCATCAAGGGTGAGAAGATCTACAAAGGCATTAGAGCCAG | 230 |
| core_34 | CAAAAATGCATTTTCGTTTGAAATACCGACCGGCTTAGGTATTGAGCG | 231 |
| core_35 | CATATATTAATCAGTAGCGAGGAACCGCCT | 232 |
| core_36 | ACCCCCAGCGATTATATTACCCAAATTCATCAACAGATGA | 233 |
| core_37 | AGCCTTTATTTCAACGCAAATTAAGCTAAAGATT | 234 |
| core_38 | ATCATACAGGCAAGGGGAACGCCATCAGCTCATTTTTT | 235 |
| core_39 | TTTAAATGCAATGCCTGAGTAATGTGTCGGGAGA | 236 |
| core_40 | ATATAAAGTATTTTTCGACAAAAGTTCATTTTTCTTCTGACCTAAA | 237 |
| core_41 | TTAAAGGTGCCAAAGACAAAAGGGCTTTTTACATTCAACCGATTGA | 238 |
| core_42 | AGGTAAATATTGTTTTTCGGAAATTATTCATGCCAGTT | 239 |
| core_43 | GAGAGGGTCCGTACTCAGGCGAAGCCCTTTTTTTTTAA | 240 |
| core_44 | CAGTCCCGGAATTTGTTTTTGAGAGATAGACTTTACGTACAGCG | 241 |
| core_45 | TTATTTATCCCCCAATAGCAAGCAAATCAGATTTTTTTAGAAGGCTTATCCG | 242 |
| core_46 | GCTAATGCAGAACGCGCATCAAGAAAACACAAGACTTTTTAAGAACGCGGTA | 243 |
| core_47 | CCACGGGATTTCATTTGATTTTTTTACCTTTTTTAATAACTAT | 244 |
| core_48 | TTTCATCGGCATTTTTTTTCGGACCAGAGCCTTTTTCCACCCAGAA | 245 |
| core_49 | TTACCAGCGAATTATCACCATTACTATTCACAATTTTTCAAATAAA | 246 |
| core_50 | AAATATCAATAAGAAACGATTTTTTTTTTGTTTAACGTACAAAATA | 247 |
| core_51 | AGCCGCCGCAGGTCAGACGATTGGCTTTTTTTGACATTAGCAAGGCCGGA | 248 |
| core_52 | AACGTAGAAAATTTTTACATACATAAAGGCAGTAT | 249 |
| core_53 | ATGTAAATTTTTTCTGATGCAAATCCAATGTTATTTTAGTTAAT | 250 |
| core_54 | GCTCATGGAGCACCAGCAGAAGATAAAACAGAGGTGACAAATCAACA | 251 |
| core_55 | GTAAAGTAATTCTGTCTTACCGCGCAATCCAACATTCCAA | 252 |
| core_56 | ATACCGGGCACATTAATTGCGTTGTCCACTATGGAAAGCCGG | 253 |
| core_57 | TCACCCAAGAGAACAAGCAAGCCGAAGTACCGCACTCATC | 254 |
| core_58 | AATGCGCGAAGAATACGTGGCACAGCACTAAAGCGCGTAA | 255 |
| core_59 | GCCAGCGGGAGGTGTCCAGCATCAATCCCGTAAAAAAAGCCGCACAG | 256 |
| core_60 | AAACTTAAAAAGAGACGCAGAAACGAAAGGATTAGGATTATGCCGTC | 257 |
| core_61 | ACATCGACATAAAAAAGCGGGGTCCAGGAACGACGTGCTT | 258 |
| core_62 | TCGGCGAACTCCGTGGTGAAGGGAAAGATGATAACCTATTGTTAATGC | 259 |
| core_63 | TGATTGCTTTGAATAGTACCTTTCCGTTGTAGCCACAGTGCCACGCT | 260 |
| core_64 | TAGTCTTTATTAAAAAAATCGTCTTTGCAACACGCCGCTAAGAATCAG | 261 |
| core_65 | CGGCAAACGCGTACGCCAGAATCACGATTTTAGA | 262 |
| core_66 | ACGTCGCGCAGAGGCGAATACAGGAGGCCGACAAATTAATACATCGG | 263 |
| core_67 | CCCTCAGTTTTCATACCCTCAGAAAATAACAGTGCCCGCCTGAACAATTACC | 264 |
| core_68 | TGAGCAAAAGTAGCTCTCACTCCTCAAGAGAAAGTAAGCAGATA | 265 |
| core_69 | AACCACCACCAGGCCACCA | 266 |
| core_70 | AGAAAAATAATCCGCGCTTAATGGGAAAAACATTAGTAA | 267 |
| core_71 | ATTTACGAGCATGTAGAGATAAGTTATAAACAATTCTGAACACCGGA | 268 |
| core_72 | GCTGGCAAGTTGCTTTGACTGCACTCTGTGGT | 269 |
| core_73 | GCTAGGGCAGCTAACTGGTTTCTGCCAGCACGTACCTGCACACGTATA | 270 |
| core_74 | TCAGTATTTAACATCACCATCACGTTAAAGGGGATTCGCCGTTGTGT | 271 |
| core_75 | AAAAACCGTCTATCAGGGCGATGGCCGAGCTCCCACCACACCCGATC | 272 |
| core_76 | CCCTCAGCCCCTGCCACTGGTAACAATAATCATTAAACC | 273 |
| core_77 | TCCTCATTAGGAGTGTGGGAGGGAGAACGGGTGGCTGTCTTTCCTTAT | 274 |
| core_78 | TGGCTTTTAACGGGGTAGGTTGAGACCCTCAGAACCGCCA | 275 |
| core_79 | TAAAGAACGTGGACTCCAACGTCAAATGAGTG | 276 |
| core_80 | TAAGTTTTGATGATACAAAGCCAGAATGGAAAGCGCAGTTTGACAGG | 277 |
| core_81 | CAGTGCCTTGAGTACCCATCCTATTTTGGGGGTGAACCA | 278 |
| core_82 | TACCGAACGAACCCGCCGCCAGCACTCTGAATTTACCTGAATGGCTAT | 279 |
| core_83 | CTACATTTTTTGTTCCAGTAAGCGTCATACAATCAAGTT | 280 |
| core_84 | ATCAAAATACATGAAAGTATATATCATTTCA | 281 |
| core_85 | AACACCGCCTGCAATCAGCCGCCACCAG | 282 |
| core_86 | GTTAGCATATTTCGGGAAACAAACCTGTTTATCAACAATAAACCAAT | 283 |
| core_87 | CCGAGTAAAAGAGTCTGTCTTGCCTGTCCTCGTTCAGGGCGC | 284 |
| core_88 | AGTAGAAGATATTACCGCCAGCCAGAAATGGAGTCACGCTTCGGAACC | 285 |
| core_89 | TCATAGCCCCCTTATCGGATAAGGCGGGGTTTTGCTCA | 286 |
| core_90 | TGAATATACAGTAACAGTACCAGGTAGCGTTTGCATAAGAGGCTGAG | 287 |
| core_91 | GGTCCGTTTTTTCGTTTTTTTCGTCGCTGGCAGCATTGCCGTTC | 288 |
| core_92 | GCCTTGCTGGTTTTTTATATCCAGAACAAACTCAA | 289 |
| core_93 | GCGCGGGGAGAGGCGGGAATGCCAACGGCTGTCACTTTTTGCGCGCCTGGAG | 290 |
| core_94 | TTATTTACGAAAGCGTAACTGATAATTTAGAAGTTTTTATTAGACT | 291 |
| core_95 | GCGGCCTTCCCACGCAACTTTTTAGCTTACGGCTGTGCCGGTG | 292 |
| core_96 | CCCCCTGCTTTTTTCAGACGATCCAGCGGCCGGCCAGAATGCGG | 293 |
| core_97 | GCTGCAACAAGGGAAGAAAGTTTTTGAAAGGAGCGGGCCTTGACGG | 294 |
| core_98 | GGAATTGGTCAATAGATAATACATTTTTTTGAGGGCCCTAAAACATCGCC | 295 |
| core_99 | CGCTCACTGCTTTTTCGCTTTCCACGGGTTTTTCGTTTTCACGGTC | 296 |
| core_100 | CGAACGTGGCGAGATAGGGTTGAGTGTTGTTCTTTTTAGTTTGGAACAAGAG | 297 |
| core_101 | ACGTCAGAGAGAAACAATACTGAGAAGTGTTTTTTTTT | 298 |
| core_102 | GAGAGCCAGCAGCTTTTTAATGTATCTGGTCTTTTTGTTGGGGCGG | 299 |
| core_103 | TCTGACCTATTGGCAGATTCACCAGTTTTTCACACGACCAGTAATA | 300 |
| core_104 | ATTCTGGCCAACTTTTTGAGATAGAACCCTATTTAGAG | 301 |
| core_105 | CACCTTGCCCTAGGGTTAGAACCACGGAACGCAAACGGC | 302 |
| core_106 | AACTCCAAAGGTGTTTAGCTATATTTTCATTTGGGGCGTTGTACCAA | 303 |
| core_107 | CATTCGCCATGTCATAAATATTCAGCCCAATACT | 304 |
| core_108 | CAGTTCCTGATTATCTTTTTCTTCGTGGTGCTGGTCTGG | 305 |
| core_109 | CGAGAAACACCAGAACGAGTAGTACACCAACC | 306 |
| core_110 | ACTAATGCTAAAACGACGATATATTCGGTCGCAAAAGGCTGAGGCTTT | 307 |
| core_111 | ACCCGTCGGATTCTCAGCCAGCTGTCTTTACTGATACTAATAGTAGT | 308 |
| core_112 | AGTAACATGGAGCGGATTAGAGCCAGGAAGGTTATCTAAA | 309 |
| core_113 | CAATCAAAAAAATCTAAAGCATAGATTTTCAAACAGA | 310 |
| core_114 | AATAAAGACGTGGGAATGCCGGACTTGATGTTTAGACTG | 311 |
| core_115 | CAGAAAACGAGAATGACCTTGCATCAAAGTTTTGACCAGACG | 312 |
| core_116 | AAAATCAGTTCATCAACGTCTGGCCTTCCTGTAATTGCGT | 313 |
| core_117 | CAAACCCTCTGAATAATGGAGACAACGTCAG | 314 |
| core_118 | GTGGCATCAATTCACCTCAAATATATATCTTT | 315 |
| core_119 | CCTTATGCGGTGGTTCCGAAATCGGAAAATCCTGTTTGAT | 316 |
| core_120 | ACGTTGGTGGATTGACCGTAATGGATATTATTTGCACGTAAGGTTTA | 317 |
| core_121 | CGCATAACAAGAGGCAAAAGAATATGCCCTGAACATTATTAC | 318 |
| core_122 | GGGGACGACGACAGTAAGTTAGCGGCGGAATCTGCAAAAG | 319 |
| core_123 | AAAAGATTTTCGAGCTTCAAAGCGCTCCTTTTAAGGAATTGGAAGAAA | 320 |
| core_124 | ACTATCGATCCTGATAACCGCAATTTGCGTATTGGGCGCCGCCTGGC | 321 |
| core_125 | CTGAAAAGAAAGCGGAATAAATCAGATAGCGTGAGTAACAAGTTTGA | 322 |
| core_126 | AGATACATAACCAAAATAGACAGCTTGATACC | 323 |
| core_127 | GTTGAAATCAATATAAAAGAAACCCTGAGAGCAGCAGGC | 324 |
| core_128 | GCTGATTGCCCTTCACCAGGGTGGAGATGATGTTATACTT | 325 |
| core_129 | TCCTCATATACCATATCAAAAATCAGTGAGGCCA | 326 |
| core_130 | ATGGTCAATAACCAGCACTAACAACTTTGCCCGAACGGTTGATTCCCA | 327 |
| core_131 | AACGAGTATGTCTGGAAGTTTCATGGACGTTGACGAGGCA | 328 |
| core_132 | GTCGGGAAACCTGTCGAATAGCCCGAGAAAGGCCACGCTG | 329 |
| core_133 | CCAAAAGGCACAGACAGCCCTCATTCGGCCTCAGGAAGATCGCACTC | 330 |
| core_134 | AGGATTAGACATTATTAATTTTAAAAGTTTGGATTTTAA | 331 |
| core_135 | CTTGAGATGGTTTAATTTCAACTTCGAAAGACTAGTAAGAGCAAGAC | 332 |
| core_136 | ATTCTGCGTTTCGCAATTTAATTGAACCAGACCCTCGTTTCCAGAGGG | 333 |
| core_137 | CGATGCTGCTCCGGCCAGAGCACATCAGCAGCTGTTTGGAGCAATTCA | 334 |
| core_138 | ATTATCATCATAGTCGGGCAACAGAACTGGCGTGAATTA | 335 |
| core_139 | TTCACCAGTGACACTATCATAACCGGAAGCATCAGAAGC | 336 |
| core_140 | TTACAAACTGCGGAACAAAGGGACGTTTGCCCAGTTGCAGCAAGCGGT | 337 |
| core_141 | CACCAGAATATCATTTAATTCGACAACTCGTATTAAATCCTAATAGA | 338 |
| core_142 | AGCATTAACATCCTTTTTATAACATAAAGCTTTTTTAATCGGCGAG | 339 |
| core_143 | ATGACCCAATCACCATCAATATGATTTTTTTTCATTGACCATTAGATACA | 340 |
| core_144 | AAGACTTCAAATTTTTATCGCGTTTTAAAAGAGGA | 341 |
| core_145 | GATAAGAGAAGTACGGGATTTAGTACCGTTCTATTTTTCTGATAAA | 342 |
| core_146 | CACTAAAACATTTTTTCATCTTTGGACATTTTTCAACCATCGCCCA | 343 |
| core_147 | TGCAACTAGTCATTTTTGCGGATGGTTTTTTTAGAGCTTAATTGCT | 344 |
| core_148 | TCAGGCTGCGCAACTTTTTTTTGGGAAGGGCGATGGCAAAGCGC | 345 |
| core_149 | GATAGAGAGTTGAGATTTAGTTTTTAATACCACATTCAACGGAACA | 346 |
| core_150 | ATTGTATCTTTTTGTTTATCAGCTTGCTCGGTTGCGCCGACAAT | 347 |
| core_151 | TGCTGTAGCTCATTTTTCATGTTTTAAATAACGAACTA | 348 |
| core_152 | TTTGTATCATCGCCTGATGTACCGTAACAGAGGTGTTTTTATTTCTTAACGA | 349 |
| core_153 | ATAATTCGCATTAAATGTGATTGAATCCCTTTTTCTCA | 350 |
| core_154 | CAGCCAGCAAACTACAACTTTTTCCTGTAGCATTCAGCCTTTA | 351 |
| core_155 | AGGTAGAAAGATCAACGTAACAAAGCTGCTCATTTTTTCAGTGAATAAGGCT | 352 |
| side1_recess_1 | ACAAAGTACAATTCCTGAGTTT | 353 |
| side1_recess_2 | CGCCACCCTCAGAAGCAGTTGGGTAACGCCAGGGTTTTCCAATAGTGAATT | 354 |
| side1_recess_3 | TATCAAAATCATAGGTCTGAGAATTGAGTTACCA | 355 |
| side1_recess_4 | CGTCACCAGTACTTTCCGGCACCGCTTCTGGTGCCTGCTGCAA | 356 |
| side1_recess_5 | TAATTACTAGAAATCAAGAATCCTGAATCTTACCGCCATTTGCAATCAAT | 357 |
| side1_recess_6 | ATCTTGACAAGAACCGGATATTCACCAAGCGCGAA | 358 |
| side1_recess_7 | GTATAAAGCCAACAGAATAAACACCGGAATCA | 359 |
| side1_protrusion_1 | GTTTAGTAGTTAAATAGCTCAACAAGAATCCTTGAAAACATAGCG | 360 |
| side1_protrusion_2 | ATAAGGCTCAACATCGCCATATGCGTTATACAAATTCTTACCA | 361 |
| side1_protrusion_3 | CCATATTTAACAAGTAATTTACTCCCGATTTCCAGAGCCTAATT | 362 |
| side1_protrusion_4 | AGAGACTATGACTGAAGACGATAACCTTGCTTCTGTAAATCGTC | 363 |
| side1_protrusion_5 | GCTATTAATTAATAACCTCCGTGTGATAACCCTGAACAGCCTTTA | 364 |
| side1_protrusion_6 | ATAGCTTAGATTAGAAGAGTCCAGTCACG | 365 |
| side1_protrusion_7 | TATGTGAGCCTTTTTTTTCCCTTGTAGGGCTTAATTGAGAATCG | 366 |
| side2_recess_1 | CTGGTGTGTTCAGCAAATCAGCGGGAGCTAATATCTTCTTTG | 367 |
| side2_recess_2 | CCAAGTTACAAAAGAAATTTCTGCTCATTTGCCGCCAGCACATCCCTTACA | 368 |
| side2_recess_3 | ATCCCCGGGTACCCACTACTCGAGGTGCCGTAAAGACAATATTGACGCTC | 369 |
| side2_recess_4 | TACATTTAACAAACGGATAACCTCACCGGAAACAAAGCGGATC | 370 |
| side2_recess_5 | GAAATTGTTATCCAGCCTCCTCACAGTTGAGG | 371 |
| side2_recess_6 | TTTTTATTTTCATCGTAGGAATCACAGACGACGAC | 372 |
| side2_recess_7 | AATAAACAACATCGAAATTAAT | 373 |
| side2_protrusion_1 | GAAGCATAAAGTGGGGTGCCTAAGGGCGCTAAAGGGAGCCCCCG | 374 |
| side2_protrusion_2 | GGTTGCGGTATGATGCCGGGTCGTGCCTGGTACTATGGTGTAGCG | 375 |
| side2_protrusion_3 | GAATTCGTCGTCCGTGGCTCACAAACTGTTGCCCTGCGGCTGGTA | 376 |
| side2_protrusion_4 | ATGGGTAAAGGTTGTCATAAAGTTGGGCG | 377 |
| side2_protrusion_5 | GTTAACGGCGCGCTCTCTTTTTTCGCACTCAATCCGCCGGGCGC | 378 |
| side2_protrusion_6 | ATTGCAGGCATCAGAGCCGGGTCTTCCACACAACATACGAGCCG | 379 |
| side2_protrusion_7 | TTCTTCGAATCCTGTAAAGCATGGTCATAGCTGTTTCCTGTGT | 380 |
| side3_recess_1 | TTTGAGGACTAAACCGCTTTTGCGGGATCGTC | 381 |
| side3_recess_2 | CGGTGGTGCCATTAGTGATGAAGGGTAAAGTTAAAGATAGGTC | 382 |
| side3_recess_3 | GAATTGCGAATAATAATTTGGTAATAGTAAATAGTATTATAG | 383 |
| side3_recess_4 | GTAGATGGGCGCATCGTAACCGTGAACAACTAAAG | 384 |
| side3_recess_5 | ACCCTCAGCAGTAATCATTTCATTATACCAGTCATCCATATAAGAGTACC | 385 |
| side3_recess_6 | GCAAAATCCCTTATAAATCAAAAGTGCCAGCTGCA | 386 |
| side3_recess_7 | TTAATGAATCGCAGAGCACCGT | 387 |
| side3_protrusion_1 | AACAGTTTCAGCGTAGAAAGGCATCTGCC | 388 |
| side3_protrusion_2 | TTTCACGTTAAAGAAGAGTGAGTTTTGTCGTCTTTCCAGACGTT | 389 |
| side3_protrusion_3 | AGCATCGGGTTAAAGGGACTTTTTGGATTTTGCTAAACAACTTTC | 390 |
| side3_protrusion_4 | CGGGTAAAATACGTACGAAGGAATTGGGAATCTACGTTAATAAA | 391 |
| side3_protrusion_5 | GCAGGGAAACCCACTAATGGAGGGTAGCAACGGCTACAGAGGC | 392 |
| side3_protrusion_6 | TAACGATCTGAAAATTCTGTATGCATGAGGAAGTTTCCATTAAA | 393 |
| side3_protrusion_7 | AGTAAATGAATTTCTCCAAAATGAGGCTTACGATAAAAACGCCAA | 394 |

TABLE 5
T = 1 (FRET)

| | | SEQ ID |
|---|---|---|
| Name | Sequence | NO: |
| core_1 | TGATATAAGTATAGCCAACCAATACAAAGAATTAATTAATATTTTGT | 395 |
| core_2 | GGAAACCACGGTGCGGGCCTCTTCAGCCCAATGTATAAGCAAAAGCCC | 396 |
| core_3 | GCTATTACTAAAATTCGCATTGCTTTAAACAGTT | 397 |
| core_4 | CCATGTTTACATAGCTATCTTACAGGAAACAATG | 398 |
| core_5 | GCCACCGCCACCCTCAGAGAGCCCAATAATACGAGGAAAGTGTATCA | 399 |
| core_6 | CGCCACCCTCAGAACCGGAATAGCGCAATAATAGGACTGTAGCGCGT | 400 |
| core_7 | AATACCCAACGGAATACAAGATAGCAGCACCG | 401 |
| core_8 | AGTGTACCCCGGTTGCTGCTCCAATTTTCAGGGATAGCA | 402 |
| core_9 | CTTATTACGTGGCAACATATAAAACATATGGTCAGAGAGACGAGCGTC | 403 |
| core_10 | TGTTACTTAGCCGCATTAGACGGAAGACACCAAAGAACT | 404 |
| core_11 | TTAATGCCAGAATCGAGAATATAAACGGTGTACCAACTTTGAAAGAGG | 405 |
| core_12 | GTAAAACGACGGCCAGTGAATCAATAAAATAGCACTAATATC* | 406 |
| core_13 | GCAGACGGTCAATCATTCCGCGACATAATCAGAAATATTTCTCAGAG | 407 |
| core_14 | AAAATAGCAAAGTCAGAGGAGAAAACTTTTTC | 408 |
| core_15 | AGCCCGAGGAAGATTAGGAACCCATAAATTGTGTCGAAAAAGGGAAC | 409 |
| core_16 | GATTATGGCGAAAGGGGGATGAATAAATTTTTGTTAAATCAAAAt | 410 |
| core_17 | GCCGAACAAAGTTACCAGTAAGACTCAGAGAGATACTGAACA | 411 |
| core_18 | GCCTTTAGCGTCACAAAGGATAAAAATTTTTAGAACCCT | 412 |
| core_19 | TCAAGTTTGGCATGATAAGGAAACAGAGCAAGTTTAGTACACGTT | 413 |
| core_20 | AATAAAGCAAATTGTAAACGCGGCCACCCTC | 414 |
| core_21 | CAAAATCAAACGTCACAGAAAATTGAAACGCAGAGAATTAAACCCACA | 415 |
| core_22 | TTTTGTCAGGATATCAGGTCATTGCCTGAGAACCCAGCT | 416 |
| core_23 | CCAGTAGCACCGTCACCGACTTGAAACGCTAAATAACATA | 417 |
| core_24 | TGGGTTATATGGAAACAGTACATACCAAGCTTTCAGAGGTGGAGCCG | 418 |
| core_25 | TGTCAATCATATTTAAACGAGGCACAATTTTTTAGTTGC | 419 |
| core_26 | AGAACGCGAGGCGTTTTAGCGAACGGCAGAGG | 420 |
| core_27 | AAACATTCAAAAACATGAACGGTCGAACTGACAGACCAG | 421 |
| core_28 | GTCTGGAGAACTAGCAGACAGTCATGTAATACTTTTGAGG | 422 |
| core_29 | AATCGTAACAAACAAGGGAGAGGGTAGCTATTTTTGAGAAGGCCGGA | 423 |
| core_30 | CTTGCGGGAGGTTTTGAAGCCTTAAAAAGCCTAAAACAGGGAAGCGG | 424 |
| core_31 | TTTAATGGAGCCAGTAATAAGAGAGTATTCTAAACAGCCATA | 425 |
| core_32 | TATTTTGCACCTTCATCAAGAGTAGCGCATAGGCTGGCTG | 426 |
| core_33 | CAATGAAACCATCAAGGGTGAGAAGATCTACAAAGGCATTAGAGCCAG | 427 |
| core_34 | CAAAAATGCATTTTCGTTTGAAATACCGACCGGCTTAGGTATTGAGCG | 428 |
| core_35 | CATATATTAATCAGTAGCGAGGAACCGCCT | 429 |
| core_36 | ACCCCCAGCGATTATATTACCCAAATTCATCAACAGATGA | 430 |
| core_37 | AGCCTTTATTTCAACGCAAATTAAGCTAAAGATT | 431 |
| core_38 | ATCATACAGGCAAGGGGAACGCCATCAGCTCATTTTTT | 432 |
| core_39 | TTTAAATGCAATGCCTGAGTAATGTGTCGGGAGA | 433 |
| core_40 | ATATAAAGTATTTTTCGACAAAAGTTCATTTTTCTTCTGACCTAAA | 434 |
| core_41 | TTAAAGGTGCCAAAGACAAAAGGGCTTTTTACATTCAACCGATTGA | 435 |
| core_42 | AGGTAAATATTGTTTTTCGGAAATTATTCATGCCAGTT | 436 |
| core_43 | GAGAGGGTCCGTACTCAGGCGAAGCCCTTTTTTTTTAA | 437 |
| core_44 | CAGTCCCGGAATTTGTTTTTGAGAGATAGACTTTACGTACAGCG | 438 |
| core_45 | TTATTTATCCCCCAATAGCAAGCAAATCAGATTTTTTTAGAAGGCTTATCCG | 439 |
| core_46 | GCTAATGCAGAACGCGCATCAAGAAAACACAAGACTTTTTAAGAACGCGGTA | 440 |
| core_47 | CCACGGGATTTCATTTGATTTTTTTACCTTTTTTAATAACTAT | 441 |
| core_48 | TTTCATCGGCATTTTTTTTCGGACCAGAGCCTTTTTCCACCCAGAA | 442 |
| core_49 | TTACCAGCGAATTATCACCATTACTATTCACAATTTTTCAAATAAA | 443 |
| core_50 | AAATATCAATAAGAAACGATTTTTTTTTTGTTTAACGTACAAAATA | 444 |
| core_51 | AGCCGCCGCAGGTCAGACGATTGGCTTTTTTTGACATTAGCAAGGCCGGA | 445 |
| core_52 | AACGTAGAAAATTTTTACATACATAAAGGCAGTAT | 446 |
| core_53 | ATGTAAATTTTTTCTGATGCAAATCCAATGTTATTTTAGTTAAT | 447 |
| core_54 | GCTCATGGAGCACCAGCAGAAGATAAAACAGAGGTGACAAATCAACA | 448 |
| core_55 | GTAAAGTAATTCTGTCTTACCGCGCAATCCAACATTCCAA | 449 |
| core_56 | ATACCGGGCACATTAATTGCGTTGTCCACTATGGAAAGCCGG | 450 |
| core_57 | TCACCCAAGAGAACAAGCAAGCCGAAGTACCGCACTCATC | 451 |
| core_58 | AATGCGCGAAGAATACGTGGCACAGCACTAAAGCGCGTAA | 452 |
| core_59 | GCCAGCGGGAGGTGTCCAGCATCAATCCCGTAAAAAAAGCCGCACAG | 453 |
| core_60 | CTTAAAAAGAGACGCAGAAACGAAAGGATTAGGATTATGCCGTC+ | 454 |
| core_61 | GTACATCGACATAAAAAAGCGGGGTCCAGGAACGACGTGCTT* | 455 |
| core_62 | TCGGCGAACTCCGTGGTGAAGGGAAAGATGATAACCTATTGTTAATGC | 456 |
| core_63 | TGATTGCTTTGAATAGTACCTTTCCGTTGTAGCCACAGTGCCACGCT | 457 |
| core_64 | TAGTCTTTATTAAAAAAATCGTCTTTGCAACACGCCGCTAAGAATCAG | 458 |
| core_65 | CGGCAAACGCGTACGCCAGAATCACGATTTTAGA | 459 |
| core_66 | ACGTCGCGCAGAGGCGAATACAGGAGGCCGACAAATTAATACATCGG | 460 |
| core_67 | CCCTCAGTTTTCATACCCTCAGAAAATAACAGTGCCCGCCTGAACAATTACC | 461 |
| core_68 | TGAGCAAAAGTAGCTCTCACTCCTCAAGAGAAAGTAAGCAGATA | 462 |
| core_69 | AACCACCACCAGGCCACCA | 463 |
| core_70 | AGAAAAATAATCCGCGCTTAATGGGAAAAACATTAGTAA | 464 |
| core_71 | ATTTACGAGCATGTAGAGATAAGTTATAAACAATTCTGAACACCGGA | 465 |
| core_72 | GCTGGCAAGTTGCTTTGACTGCACTCTGTGGT | 466 |
| core_73 | GCTAGGGCAGCTAACTGGTTTCTGCCAGCACGTACCTGCACACGTATA | 467 |
| core_74 | TCAGTATTTAACATCACCATCACGTTAAAGGGGATTCGCCGTTGT | 468 |
| core_75 | AAAAACCGTCTATCAGGGCGATGGCCGAGCTCCCACCACACCCGATC | 469 |
| core_76 | CCCTCAGCCCCTGCCACTGGTAACAATAATCATTAAACC | 470 |
| core_77 | TCCTCATTAGGAGTGTGGGAGGGAGAACGGGTGGCTGTCTTTCCTTAT | 471 |
| core_78 | TGGCTTTTAACGGGGTAGGTTGAGACCCTCAGAACCGCCA | 472 |
| core_79 | TAAAGAACGTGGACTCCAACGTCAAATGAGTG | 473 |
| core_80 | TAAGTTTTGATGATACAAAGCCAGAATGGAAAGCGCAGTTTGACAGG | 474 |
| core_81 | CAGTGCCTTGAGTACCCATCCTATTTTGGGGGTGAACCA | 475 |
| core_82 | TACCGAACGAACCCGCCGCCAGCACTCTGAATTTACCTGAATGGCTAT | 476 |
| core_83 | CTACATTTTTTGTTCCAGTAAGCGTCATACAATCAAGTT | 477 |
| core_84 | ATCAAAATACATGAAAGTATATATCATTTCA | 478 |
| core_85 | AACACCGCCTGCAATCAGCCGCCACCAG | 479 |
| core_86 | GTTAGCATATTTCGGGAAACAAACCTGTTTATCAACAATAAACCAAT | 480 |
| core_87 | CCGAGTAAAAGAGTCTGTCTTGCCTGTCCTCGTTCAGGGCGC | 481 |
| core_88 | AGTAGAAGATATTACCGCCAGCCAGAAATGGAGTCACGCTTCGGAACC | 482 |
| core_89 | TCATAGCCCCCTTATCGGATAAGGCGGGGTTTTGCTCA | 483 |
| core_90 | TGAATATACAGTAACAGTACCAGGTAGCGTTTGCATAAGAGGCTGAG | 484 |
| core_91 | GGTCCGTTTTTTCGTTTTTTTCGTCGCTGGCAGCATTGCCGTTC | 485 |
| core_92 | GCCTTGCTGGTTTTTTATATCCAGAACAAACTCAA | 486 |
| core_93 | GCGCGGGGAGAGGCGGGAATGCCAACGGCTGTCACTTTTTGCGCGCCTGGAG | 487 |
| core_94 | TTATTTACGAAAGCGTAACTGATAATTTAGAAGTTTTTATTAGACT | 488 |
| core_95 | GCGGCCTTCCCACGCAACTTTTTAGCTTACGGCTGTGCCGGTG | 489 |
| core_96 | CCCCCTGCTTTTTTCAGACGATCCAGCGGCCGGCCAGAATGCGG | 490 |
| core_97 | GCTGCAACAAGGGAAGAAAGTTTTTGAAAGGAGCGGGCCTTGACGG | 491 |
| core_98 | GGAATTGGTCAATAGATAATACATTTTTTTGAGGGCCCTAAAACATCGCC | 492 |
| core_99 | CGCTCACTGCTTTTTCGCTTTCCACGGGTTTTTCGTTTTCACGGTC | 493 |
| core_100 | CGAACGTGGCGAGATAGGGTTGAGTGTTGTTCTTTTTAGTTTGGAACAAGAG | 494 |
| core_101 | ACGTCAGAGAGAAACAATACTGAGAAGTGTTTTTTTTT | 495 |
| core_102 | GAGAGCCAGCAGCTTTTTAATGTATCTGGTCTTTTTGTTGGGGCGG | 496 |
| core_103 | TCTGACCTATTGGCAGATTCACCAGTTTTTCACACGACCAGTAATA | 497 |
| core_104 | ATTCTGGCCAACTTTTTGAGATAGAACCCTATTTAGAG | 498 |
| core_105 | CACCTTGCCCTAGGGTTAGAACCACGGAACGCAAACGGC | 499 |
| core_106 | AACTCCAAAGGTGTTTAGCTATATTTTCATTTGGGGCGTTGTACCAA | 500 |
| core_107 | CATTCGCCATGTCATAAATATTCAGCCCAATACT | 501 |
| core_108 | CAGTTCCTGATTATCTTTTTCTTCGTGGTGCTGGTCTGG | 502 |
| core_109 | CGAGAAACACCAGAACGAGTAGTACACCAACC | 503 |
| core_110 | ACTAATGCTAAAACGACGATATATTCGGTCGCAAAAGGCTGAGGCTTT | 504 |
| core_111 | ACCCGTCGGATTCTCAGCCAGCTGTCTTTACTGATACTAATAGTAGT | 505 |
| core_112 | AGTAACATGGAGCGGATTAGAGCCAGGAAGGTTATCTAAA | 506 |
| core_113 | CAATCAAAAAAATCTAAAGCATAGATTTTCAAACAGA | 507 |
| core_114 | AATAAAGACGTGGGAATGCCGGACTTGATGTTTAGACTG | 508 |
| core_115 | CAGAAAACGAGAATGACCTTGCATCAAAGTTTTGACCAGACG | 509 |
| core_116 | AAAATCAGTTCATCAACGTCTGGCCTTCCTGTAATTGCGT | 510 |
| core_117 | CAAACCCTCTGAATAATGGAGACAACGTCAG | 511 |
| core_118 | GTGGCATCAATTCACCTCAAATATATATCTTT | 512 |
| core_119 | CCTTATGCGGTGGTTCCGAAATCGGAAAATCCTGTTTGAT | 513 |
| core_120 | TTGGTGGATTGACCGTAATGGATATTATTTGCACGTAAGGTTTAt | 514 |
| core_121 | CGCATAACAAGAGGCAAAAGAATATGCCCTGAACATTATTAC | 515 |
| core_122 | GAGGGGACGACGACAGTAAGTTAGCGGCGGAATCTGCAAAAG* | 516 |
| core_123 | AAAAGATTTTCGAGCTTCAAAGCGCTCCTTTTAAGGAATTGGAAGAAA | 517 |
| core_124 | ACTATCGATCCTGATAACCGCAATTTGCGTATTGGGCGCCGCCTGGC | 518 |
| core_125 | CTGAAAAGAAAGCGGAATAAATCAGATAGCGTGAGTAACAAGTTT | 519 |
| core_126 | AGATACATAACCAAAATAGACAGCTTGATACC | 520 |
| core_127 | GTTGAAATCAATATAAAAGAAACCCTGAGAGCAGCAGGC | 521 |
| core_128 | GCTGATTGCCCTTCACCAGGGTGGAGATGATGTTATACTT | 522 |
| core_129 | TCCTCATATACCATATCAAAAATCAGTGAGGCCA | 523 |
| core_130 | ATGGTCAATAACCAGCACTAACAACTTTGCCCGAACGGTTGATTCCCA | 524 |
| core_131 | AACGAGTATGTCTGGAAGTTTCATGGACGTTGACGAGGCA | 525 |
| core_132 | GTCGGGAAACCTGTCGAATAGCCCGAGAAAGGCCACGCTG | 526 |
| core_133 | CCAAAAGGCACAGACAGCCCTCATTCGGCCTCAGGAAGATCGCACTC | 527 |
| core_134 | AGGATTAGACATTATTAATTTTAAAAGTTTGGATTTTAA | 528 |
| core_135 | CTTGAGATGGTTTAATTTCAACTTCGAAAGACTAGTAAGAGCAAGAC | 529 |
| core_136 | ATTCTGCGTTTCGCAATTTAATTGAACCAGACCCTCGTTTCCAGAGGG | 530 |
| core_137 | CGATGCTGCTCCGGCCAGAGCACATCAGCAGCTGTTTGGAGCAATTCA | 531 |
| core_138 | ATTATCATCATAGTCGGGCAACAGAACTGGCGTGAATTA | 532 |
| core_139 | TTCACCAGTGACACTATCATAACCGGAAGCATCAGAAGC | 533 |
| core_140 | TTACAAACTGCGGAACAAAGGGACGTTTGCCCAGTTGCAGCAAGCGGT | 534 |
| core_141 | CACCAGAATATCATTTAATTCGACAACTCGTATTAAATCCTAATAGA | 535 |
| core_142 | AGCATTAACATCCTTTTTATAACATAAAGCTTTTTTAATCGGCGAG | 536 |
| core_143 | ATGACCCAATCACCATCAATATGATTTTTTTTCATTGACCATTAGATACA | 537 |
| core_144 | AAGACTTCAAATTTTTATCGCGTTTTAAAAGAGGA | 538 |
| core_145 | GATAAGAGAAGTACGGGATTTAGTACCGTTCTATTTTTCTGATAAA | 539 |
| core_146 | CACTAAAACATTTTTTCATCTTTGGACATTTTTCAACCATCGCCCA | 540 |
| core_147 | TGCAACTAGTCATTTTTGCGGATGGTTTTTTTAGAGCTTAATTGCT | 541 |
| core_148 | TCAGGCTGCGCAACTTTTTTTTGGGAAGGGCGATGGCAAAGCGC | 542 |
| core_149 | GATAGAGAGTTGAGATTTAGTTTTTAATACCACATTCAACGGAACA | 543 |
| core_150 | ATTGTATCTTTTTGTTTATCAGCTTGCTCGGTTGCGCCGACAAT | 544 |
| core_151 | TGCTGTAGCTCATTTTTCATGTTTTAAATAACGAACTA | 545 |
| core_152 | TTTGTATCATCGCCTGATGTACCGTAACAGAGGTGTTTTTATTTCTTAACGA | 546 |
| core_153 | ATAATTCGCATTAAATGTGATTGAATCCCTTTTTCTCA | 547 |
| core_154 | CAGCCAGCAAACTACAACTTTTTCCTGTAGCATTCAGCCTTTA | 548 |
| core_155 | AGGTAGAAAGATCAACGTAACAAAGCTGCTCATTTTTTCAGTGAATAAGGCT | 549 |
| side1_recess_1 | ACAAAGTACAATTCCTGAGTTT | 550 |
| side1_recess_2 | CGCCACCCTCAGAAGCAGTTGGGTAACGCCAGGGTTTTCCAATAGTGAATT | 551 |
| side1_recess_3 | TATCAAAATCATAGGTCTGAGAATTGAGTTACCA | 552 |
| side1_recess_4 | CGTCACCAGTACTTTCCGGCACCGCTTCTGGTGCCTGCTGCAAGGC | 553 |
| side1_recess_5 | TAATTACTAGAAATCAAGAATCCTGAATCTTACCGCCATTTGCAATCAAT | 554 |
| side1_recess_6 | ATCTTGACAAGAACCGGATATTCACCAAGCGCGAA | 555 |
| side1_recess_7 | GTATAAAGCCAACAGAATAAACACCGGAATCA | 556 |
| side1_protrusion_1 | GTTTAGTAGTTAAATAGCTCAACAAGAATCCTTGAAAACATAGCG | 557 |
| side1_protrusion_2 | ATAAGGCTCAACATCGCCATATGCGTTATACAAATTCTTACCA | 558 |
| side1_protrusion_3 | CCATATTTAACAAGTAATTTACTCCCGATTTCCAGAGCCTAATT | 559 |
| side1_protrusion_4 | AGAGACTATGACTGAAGACGATAACCTTGCTTCTGTAAATCGTC | 560 |
| side1_protrusion_5 | GCTATTAATTAATAACCTCCGTGTGATAACCCTGAACAGCCTTTA | 561 |
| side1_protrusion_6 | ATAGCTTAGATTAGAAGAGTCCAGTCACG | 562 |
| side1_protrusion_7 | TATGTGAGCCTTTTTTTTCCCTTGTAGGGCTTAATTGAGAATCG | 563 |
| side2_recess_1 | CTGGTGTGTTCAGCAAATCAGCGGGAGCTAATATCTTCTTTG | 564 |
| side2_recess_2 | CCAAGTTACAAAAGAAATTTCTGCTCATTTGCCGCCAGCACATCCCTTACA | 565 |
| side2_recess_3 | ATCCCCGGGTACCCACTACTCGAGGTGCCGTAAAGACAATATTGACGCTC | 566 |
| side2_recess_4 | TACATTTAACAAACGGATAACCTCACCGGAAACAAAGCGGATCAAA | 567 |
| side2_recess_5 | GAAATTGTTATCCAGCCTCCTCACAGTTGAGG | 568 |
| side2_recess_6 | TTTTTATTTTCATCGTAGGAATCACAGACGACGAC | 569 |
| side2_recess_7 | AATAAACAACATCGAAATTAAT | 570 |
| side2_protrusion_1 | GAAGCATAAAGTGGGGTGCCTAAGGGCGCTAAAGGGAGCCCCCG | 571 |
| side2_protrusion_2 | GGTTGCGGTATGATGCCGGGTCGTGCCTGGTACTATGGTGTAGCG | 572 |
| side2_protrusion_3 | GAATTCGTCGTCCGTGGCTCACAAACTGTTGCCCTGCGGCTGGTA | 573 |
| side2_protrusion_4 | ATGGGTAAAGGTTGTCATAAAGTTGGGCG | 574 |
| side2_protrusion_5 | GTTAACGGCGCGCTCTCTTTTTTCGCACTCAATCCGCCGGGCGC | 575 |
| side2_protrusion_6 | ATTGCAGGCATCAGAGCCGGGTCTTCCACACAACATACGAGCCG | 576 |
| side2_protrusion_7 | TTCTTCGAATCCTGTAAAGCATGGTCATAGCTGTTTCCTGTGT | 577 |
| side3_recess_1 | TTTGAGGACTAAACCGCTTTTGCGGGATCGTC | 578 |
| side3_recess_2 | CGGTGGTGCCATTAGTGATGAAGGGTAAAGTTAAAGATAGGTCACG | 579 |
| side3_recess_3 | GAATTGCGAATAATAATTTGGTAATAGTAAATAGTATTATAG | 580 |
| side3_recess_4 | GTAGATGGGCGCATCGTAACCGTGAACAACTAAAG | 581 |
| side3_recess_5 | ACCCTCAGCAGTAATCATTTCATTATACCAGTCATCCATATAAGAGTACC | 582 |
| side3_recess_6 | GCAAAATCCCTTATAAATCAAAAGTGCCAGCTGCA | 583 |
| side3_recess_7 | TTAATGAATCGCAGAGCACCGT | 584 |
| side3_protrusion_1 | AACAGTTTCAGCGTAGAAAGGCATCTGCC | 585 |
| side3_protrusion_2 | TTTCACGTTAAAGAAGAGTGAGTTTTGTCGTCTTTCCAGACGTT | 586 |
| side3_protrusion_3 | AGCATCGGGTTAAAGGGACTTTTTGGATTTTGCTAAACAACTTTC | 587 |
| side3_protrusion_4 | CGGGTAAAATACGTACGAAGGAATTGGGAATCTACGTTAATAAA | 588 |
| side3_protrusion_5 | GCAGGGAAACCCACTAATGGAGGGTAGCAACGGCTACAGAGGC | 589 |
| side3_protrusion_6 | TAACGATCTGAAAATTCTGTATGCATGAGGAAGTTTCCATTAAA | 590 |
| side3_protrusion_7 | AGTAAATGAATTTCTCCAAAATGAGGCTTACGATAAAAACGCCAA | 591 |

TABLE 6
T = 1 (−5°)

| | | SEQ ID |
|---|---|---|
| Name | Sequence | NO: |
| core_1 | CGAACGTGGCGTTTTTGAAAGGAACGCTGCGCGTTTTTAACCACCTAAAG | 592 |
| core_2 | TCACGTTGGTGTACAAGCTTTTTTTCAGAGGTGGTGGCCAGGGT | 593 |
| core_3 | CCAATAACGCGTTTTGATTGCATATCCCCCTCTGTAGCCTTAAAAG | 594 |
| core_4 | AATATTACCGCCGCGCAGCAGCATTGGGAAGTGGCTCAT | 595 |
| core_5 | GCGCTTAGAAATACCAGATAGGGCCGTCGG | 596 |
| core_6 | TGAGGAAGGTTATCTAATTTTTATATCTTTAGGAGCACTATGCAAATCAA | 597 |
| core_7 | GCAAGTGTAAATGGATTATTCCCTCCGTGGG | 598 |
| core_8 | AGGGAAGAAAGCGAAATAGAACCAAGGGACATTCTGGC | 599 |
| core_9 | ACAAGAGTCCTTTTTCTATTAAAGAAAGTGTCAC | 600 |
| core_10 | AGCTTTCATCAACATGCCATCAATTAAGAGGGGGGGCAAAGAATTAGCAA | 601 |
| core_11 | CGTGGACTAAATCCCTTATAAATTTTTTAAAAGAATAGCCCG | 602 |
| core_12 | CAGTTTTGGGGCGCGAGATTGCTGATGCTCCTTCTTTTGCAAATACTGC | 603 |
| core_13 | TTGTAATATCCAGAACAAGTGTTTGCTACAGGGCGCGTTT | 604 |
| core_14 | TTTAGAAGTAACAACTAATAGATTACCGTAATGACGCTCACGCTCATG | 605 |
| core_15 | CTGAAAAGGTGGCGGTACGCCAGAACATCACTTGCCTATAACCTGTTT | 606 |
| core_16 | GACAACTCATAATACATTTGAGGACCCCGACCAGTAATAACTTCTGACCT | 607 |
| core_17 | CTCAATCAATTGCATCTGCCAGTTTTTTTGAGGGGACGACTTTCCCAG | 608 |
| core_18 | AGAGCCGTTTCACCAGTCACAGCTTTCCAGTCGGGA | 609 |
| core_19 | AGGCAACTATAACGTGCAGGAGGCCGATTAAAGGGATACTATGG | 610 |
| core_20 | TTTAACCAATAGGAACCAACAGAGAGGAGCGGAAGTACATTGGCAGA | 611 |
| core_21 | CAAGTAACAACCCGTCGGAAATATTCATTGACAAAAAGAAAATAATT | 612 |
| core_22 | AACGGCCACACGGTCATACTTTTTGGGGGTTTCTGCTGCGCGCC | 613 |
| core_23 | TGGTGCCGGGCCGTTTTGAGCACATCCTCATAA | 614 |
| core_24 | TAGTAAATGAATTTTCATTTTGCGGAACAAAGAAACCACCA | 615 |
| core_25 | ACCGCAAGAATGCCAATGTAGAACAGGAAAAAATCGTCTGAGCGGTC | 616 |
| core_26 | AACACTATAGGGGGTAATATATATTCGGTCG | 617 |
| core_27 | TCACGACGTTTTTTGTAAAACGACGGCCAGCGGATAACCTCACCGG | 618 |
| core_28 | GTAAGAGCCCAAGCGCCCTCAGCAGCGAAAGAATTTCTTAAAATGTTT | 619 |
| core_29 | TATAGTCAGAAGCAAAGCGAATTCGAGTAGCGTCCAAAGAAGT | 620 |
| core_30 | CTTCAAAGAGAGAGTACCTTTAATATATAATGTCGTTTACACTAACGG | 621 |
| core_31 | AGAGCGGGATAGTAGTAGCATTATTTCAAC | 622 |
| core_32 | CGGGCAACTACATTTTGGGATAGGCGGAACGTGCCGGACTCGGCAGCA | 623 |
| core_33 | TTTGAGTAACATTATCTGTATGGGTTAAACAGAGACTGGA | 624 |
| core_34 | AGGTCATTAACATCAATTCTACTAAGCTAAACTTTCCTCGTTAGAATC | 625 |
| core_35 | TTTAGCCATTGCAACGTCAGCGTAACAAACGGCGGATTG | 626 |
| core_36 | CTGTGGCCGGGCGCGGTTGCGGTATTTTTTAGCCGGGTCACT | 627 |
| core_37 | AGCTGATTGCTTTTTCTTCACCGCCTCAGTGAG | 628 |
| core_38 | CATTGTGAATTACCTTATGCGATTTTTTCATGGATAAAAACCAAGGT | 629 |
| core_39 | GTTGTTCCAGTTTGGACGGCATCCCACGCAAC | 630 |
| core_40 | AAAGCGTAAGAATTAGTCTTTAATTAATTGCGTTGTTTTTGCTCACTG | 631 |
| core_41 | GGTGCTGGTCTAATAGCGAGAGGTTGATAAGTCAAATATATCATAC | 632 |
| core_42 | ATCGTCACGAAACAAAGTACAACGAGTAAATTGTTGAGA | 633 |
| core_43 | TCGGCCTTGCTGGAGTCAATACTTCTTTGATTAGTAATAATCCTGAG | 634 |
| core_44 | AATCGGCACCAACGTCTGTCCATCACGCAATTTTTTTAACCGTTGTAG | 635 |
| core_45 | GATTGCCGTTTTTTCCGGCAAAACATTTTTTCGGCGAAACGT | 636 |
| core_46 | GATGGCTTTCAGAGTAGAAGAACTCAAACTATCAGGACG | 637 |
| core_47 | TTGCTTTGACGAGCACGTAGGGCGCTGTAGACAGG | 638 |
| core_48 | ATGCGCCTTATAATCAGTGAGGCTTTTTACCGAGTAA | 639 |
| core_49 | CAGATGATGGCAATTCAGAAAACGAGTTCCAAATGCT | 640 |
| core_50 | AGCTATATTTTCACAACCATTAGATACATTTCTAAAACGACAGACGAC | 641 |
| core_51 | AACGCGGTCCGTTTTTTCAATCCGTGCTGCGGCCAGAATG | 642 |
| core_52 | TATACCAGGTGTCCAGCATCAGCGCAGCTTACGGCTGGAG | 643 |
| core_53 | GGGCTTGAGATGGTTTAATTTCAACGATTATA | 644 |
| core_54 | TTTTCAGGGATAGCACACCCTCAAAAGGTGGTACCCACCACCGGAACCGC | 645 |
| core_55 | CCTAATTTTATTCATTACCCAAATCTTGACAAGAACCGGA | 646 |
| core_56 | ATCCTGAATCTTACCAACGCTAACAACGCCAAAGAGAGATAACCAGG | 647 |
| core_57 | TAGTTGCTATTTTGCACCCAGCTAGCTAATGC | 648 |
| core_58 | TATCAGGTCATTGGTTCTAGCTGATAAATTAATGCCGGAAATGTGTA | 649 |
| core_59 | GAGATTTGTTTTTATCATCGAAAGGTTTTTCGCTTTTGCGGG | 650 |
| core_60 | CCTGATAAATTGTGTCGTGAATAAATACCACATTCAACTA | 651 |
| core_61 | AGAGCATAAAGCTAACGCATTAAAACGTTAATATTTTG | 652 |
| core_62 | TATGCGTTCCTGTTTATCAACAATATCAAGATAATAGCA | 653 |
| core_63 | AATTAAGCAATTTTTTAAGCCTCTTTGCGGGAGTTTTTAGCCTTTAACAT | 654 |
| core_64 | CACAGACAGCCCTGCCCACTTTTTCATAACCGAGTAAACAGCTT | 655 |
| core_65 | TAGTTGCGCTTTTTGACAATGACAACAACCTTACTTGCAGGGAGTT | 656 |
| core_66 | TGAGGCTTCGCCAAAAGGATTTTTTTACGAGGCATAATTCATCA | 657 |
| core_67 | CATAGCGATAGCTTAGTAACGGATTCGCCTGATTGCTTTGA | 658 |
| core_68 | AAGGAGCGGATAAAGTTTTGTCTTTTTTCTTTCCAGACGTGATACCGA | 659 |
| core_69 | CTGTAGCTTAGTTTGAATCACCATCAATATTTTTTATATTCAACCGTT | 660 |
| core_70 | CTTTAGCGTCAGACCAAGCAGCACCGTAATCAATCCCAATCTAATATC | 661 |
| core_71 | GATTTTCAATATCAAAATTATTTGAGGTATTTAAATTGTAATTTTTGTTA | 662 |
| core_72 | GAACCTGAGAGTCTGGGGTAAAGATTTAGAACCCTCATAT | 663 |
| core_73 | AGCAAACAAGAGTAAACAGATGACAAAATAATTCCAGAG | 664 |
| core_74 | CGTTCTGTAGCGCGTTTTTCATTAAACATTCAAGTTAAGCCTTTTAAGA | 665 |
| core_75 | TCATCGGCATTTTAATGCCTGAGTGAGGGTAGCTATTATCAAGTTTGC | 666 |
| core_76 | ACATCGGGAGAAACAAATTAAGACCGAGGAAAATCTTACC | 667 |
| core_77 | CAAAATCGACACCACTAGAAAATGTTACCAGCACCCTCAACCTTTT | 668 |
| core_78 | ATATTGACAGATTTGAGAGATCTACAAAGGCGCCAGTTA | 669 |
| core_79 | AGTAGATTCAACATGTTTTAAATTTTTTTGCAACTAAAGTAC | 670 |
| core_80 | AAAAATTTTCAAAAGGGTGAGAATTTTTGGCCGGAGA | 671 |
| core_81 | CAAGGATCATGTCAAGGTGTCTGGCTGGCT | 672 |
| core_82 | CACGTAAATTCTGAATAATGGAAGAACGCCTGACCCCGGTAAAACTAG | 673 |
| core_83 | CCACCCTCAGAACCGCTTAAAATTATCGGTTGCAAAAAACAGGAAGA | 674 |
| core_84 | TTAGGAGGCTTGCCCTGACGAGAAATTTTTACCAGAACGAGT | 675 |
| core_85 | CACCCTGAAGAACGCGATACAAATTCTTACCATAATTTCACAATAGCT | 676 |
| core_86 | CAAACTCCAATTTTTAGGTCAGGATTCGAACCA | 677 |
| core_87 | AGGAATCGATGAACGAACTGACCCACCAGTACAAACTAC | 678 |
| core_88 | TTTATTTTCAAAGACAAAAGGGCGAGGTGAATATTGAGCGCCAAATAA | 679 |
| core_89 | ACCGGAACCAGAGCAAAAACATTAATTTTAAA | 680 |
| core_90 | TGACCCTGAGAAAAGCCCCACATAGTTTCGT | 681 |
| core_91 | TTCATTTCAATTACGCAATAATAACGCACAAGGAAAC | 682 |
| core_92 | TTCATCAATATAATCCTTTTTTATTGTTTGGATTATACACATCCTGATTA | 683 |
| core_93 | GGTTAGAATTGTATAAGCAAATCTTTACCCTGACTA | 684 |
| core_94 | CCTCCCATGTACCGTAACATAGCCGAACAAAACATACATGAACCGCC | 685 |
| core_95 | TCATTCCATATAACAGATGCATCAAGAGTAAT | 686 |
| core_96 | GGGAGGGATGCCGGTCATATAGCGTTTGCCATCTTTTAGCATTGAC | 687 |
| core_97 | ACGGTGTACAGACCAGGGGAACCGGTAATCGTTGATAATCTAATACT | 688 |
| core_98 | AACTTTGAAAGCACAAGAATTGACCGATTGAAACGCAAA | 689 |
| core_99 | ATCAGCTCATTTCGCGTCTGGCCAATGACCATAAATTTTTCAAAAATC | 690 |
| core_100 | TTGATTCCCATTTTTTTCTGCGAACGGTAGAAAG | 691 |
| core_101 | GACCTTCAGATACATAAAGCCGGAACGAGGCGC | 692 |
| core_102 | ACAAAGTCAAACAATGAAAAGAATAAACACC | 693 |
| core_103 | CGCAGTATGTTAGCAAACGGGAATAAGGAAGCCCTCAATAATA | 694 |
| core_104 | ACCGGAAGTCATATGTTAGCATTCAGACGGTCAATCATAAGCGCATAG | 695 |
| core_105 | ACATTTAAGCGGATAAGTGCCTTAAGACTCCTTATT | 696 |
| core_106 | GGAGCCCCGCCAGGGTTAATGAATAAAGCCTGCGAACTGCAAATGA | 697 |
| core_107 | CTTTTCACGGCCCTGAGAGAGTTGGGTTCCGATCCGTGAGCCCCTGCA | 698 |
| core_108 | TCGGCCAAAATGGCTATACGTGGCACAGACAAGGAATAGG | 699 |
| core_109 | CAGCACGCTGAAGGGTATGTTTACCAGTCCCGTGGGTAACTGAAATTG | 700 |
| core_110 | ACCTGTCGTGCCAGCTGCATGGTTTTTTCACAATTTCGTAATC | 701 |
| core_111 | TATAGCCCCGCCATTATTTGAATTACCAAGCATAAAGTG | 702 |
| core_112 | CTGGTTTGACAGTGCCGTAAAGCACTAAATCGGAACCCACACCCGC | 703 |
| core_113 | CCCTATTATTCTGAAGTATTAAAAAACAGTACATAAATC | 704 |
| core_114 | ACAAGCAAGCCGTTTTAAGAACGGACATGAAAATTAGGAT | 705 |
| core_115 | AATTCATATGTTTTTTTTACCAGCGCGTCACAA | 706 |
| core_116 | CCGATTTAGAGCTCACCCTCAGAGTGATATTC | 707 |
| core_117 | CCGCCACCTAGCGGGGTTTTAGGTTTAATGG | 708 |
| core_118 | AGAACCACTCAGAACCGCCACCTGTATCACATATAAG | 709 |
| core_119 | GGAGGTTAGGCTGAGCCAGCAAATCGTAGG | 710 |
| core_120 | CAGGCGAAGGCGTGCCTTGAGTAACAGTGCCATCAGATG | 711 |
| core_121 | TATTTCGGAACCCAGCATCGAGACCGGGTTAGCAAATCG | 712 |
| core_122 | ATCAATGCCCCCTGCCAAGCCAGAGTCAGACGATTGGCCT | 713 |
| core_123 | TATCACCGCCATCGATGTAAGCGTCATACATTTTTGGCTTTTGATGAT | 714 |
| core_124 | GAGGCAGATGGAAAGCGCAGTCTTTTTTTGAATTTAC | 715 |
| core_125 | TTTTGGGGTCGAGAACAAATAAATTTTAACGGGGTCAGATGGCCCACT | 716 |
| core_126 | AAGACCCAAATCAAGTTTTGATGGTCAGCAAGCGCTCGAATCCACACAA | 717 |
| core_127 | ACGTGAACCATCAGTCAAAGGGCGAAAAACCGGCCGGTGCCCTCCTCA | 718 |
| core_128 | CAATAGAAACTCCTCATGAGTGAACTTTCCTTATCATTCCTATTTTCA | 719 |
| core_129 | GGTTTAGTACCGACCCTCAGAGCGAATACCCAAAATTTTTAACTGGCA | 720 |
| core_130 | CTCAGAGCGAGGCTCAGTACCAGCAATTTCAAAAATACC | 721 |
| core_131 | GTGCCTGTAGCTGTTTCCTGAGCCGCCACGG | 722 |
| core_132 | CGTATAAACAGTTACCACAGGAGTGTACTGGTAATAAGTCCTCATTA | 723 |
| core_133 | AGTAGCACCATTACCAAAATACCGCGCCCAAT | 724 |
| core_134 | TTAGCAAGGCTTTTTGGAAACGTCACTAACGTCA | 725 |
| core_135 | CCAAGTACCGCCCCGGGTACCGAGGTCCACGGTATTGGG | 726 |
| core_136 | TTAACGGCATAGAAGGCTTATCCGAGCAAGCAAATCAGAT | 727 |
| core_137 | AAAATCTAAAGCATCAGGAAGATCTAATGAGTTTATCCGC | 728 |
| core_138 | GAATCCCACATTAGACGGGTTTTTGAATTAACTGAAAAAATGAA | 729 |
| core_139 | AGATAAGTTTTTTCTGAACAAAAGCTTTTTTGTTTAGTATCA | 730 |
| core_140 | CGTCATAAACATCCCTTACACTGGAAGAGACGCAGTTGAGGATCACT | 731 |
| core_141 | AGTTGAAAGGAATGAGCTAACTCACATGCGGGGTGCC | 732 |
| core_142 | GACAGTATCGGCCTCACCTTGCTGAACCTCAAATATCAAAC | 733 |
| core_143 | TTAACACCGAACGAACCACCAGCATGAGTCGAGAGGGTTGCGTACTCAGG | 734 |
| core_144 | AATCATACAGGGAAGCGATCAATAATCGGCTGT | 735 |
| core_145 | TACCAAGTTATAATTTTCCCTTTTTTTGAATCCTTGAAAACCTAAATT | 736 |
| core_146 | CAATGAAATCACCGACTTGAGCCTTTTTTTTGGGAATTAGAG | 737 |
| core_147 | CTCCCTCAGAGTTTTTCGCCACCCCACCAGAGCTTTTTGCCGCCCATAAT | 738 |
| core_148 | ACAGCGCCAAAGTTAAACGATGCTGTTGCCCTTGTGCAC | 739 |
| core_149 | CCTGAGCAAAAGAAGATTTTTTATGAAACAAACATCAAAACAGGCGAATT | 740 |
| core_150 | CCTTTACTCCCGACTTGCGGGAGGTTTTTTTTGAAGCCTTAA | 741 |
| core_151 | GAAGATAAGAAAACAAAATTAATTAATATATGAGAGAAGGGTATTAAG | 742 |
| core_152 | TAACCTTGCTTCTAATAAGTTTTTCGTTAAATATAGTCTTCTGA | 743 |
| core_153 | TAATGGTTTTTTTTAAATACCGACCGTGTGAAAATAATTACTAGAA | 744 |
| core_154 | GCGGCTGGTAATGGGTAAAGGTTTTTTAGTGA | 745 |
| core_155 | ATAGCCCTAAAACATTATTTTTGCGCGCGGGCACTGACGGGGAAAGCCGG | 746 |
| core_156 | AGAAAAATAATATCCCTAGCGAACCAGAGAGAATAACATA | 747 |
| side1_recess_1 | CATCGTAACCGATCTGGTCAGTTGTAGACTTTACAAACAATTC | 748 |
| side1_recess_2 | CCACTACGAAGGCCGGCTACAGAGGCTTTGAG | 749 |
| side1_recess_3 | TAAATGTGAGCGATAGGTATTAAATCCTTTGCCCGAACGTGCTCCAAAAGG | 750 |
| side1_recess_4 | GGGTCATTGCAGGCGCTTTCGCACTCGTCTCGTCG | 751 |
| side1_recess_5 | CTGGCAGCCTCTGCGATGGGCG | 752 |
| side1_recess_6 | GACTAAAGACTTTAAGAACAAAAATCTACGTTAAGCAAATGGAGAGCTTA | 753 |
| side1_recess_7 | AGCCTTTAATTGTATCGGTGGAATCGTCATATTCGAAAGACT | 754 |
| side1_protrusion_1 | AGGAAGTTGGTAGCAAACCAACCTAAAGGAATTGCGAATAATAAT | 755 |
| side1_protrusion_2 | TTTTTCACGTTGAAAAAAAAGTATTAATT | 756 |
| side1_protrusion_3 | TTATCAGCAAATCCAAAATCCAACTTTCAACAGTTTCAGCGGAG | 757 |
| side1_protrusion_4 | ATTTTGCTTTGCTTTGAACAACTAAAACGAAAGAGGCAAAAGAA | 758 |
| side1_protrusion_5 | GAACGAGTCCTTTGTCATCATTAAACGGGTAAAATACGTAATG | 759 |
| side1_protrusion_6 | TGAGAATAGAAAGCGAGGTGACAGCATCGTTTGCCAGCATAACCC | 760 |
| side1_protrusion_7 | TACACTAAAACACACCCCCAGCTTTAATAACAACATTATTACAG | 761 |
| side2_recess_1 | CAACGTAACAAAGCTGCTCATTCAGAAATCCGCGA | 762 |
| side2_recess_2 | CCATATTTAACGAGCGTCTACAGCCATATTATTTGTAGCGACGGAAATTA | 763 |
| side2_recess_3 | CCTGCTCCATGATCCATAGTTA | 764 |
| side2_recess_4 | AAGACAAAGAACGCGAGAAAAAGTAAGCAGACTGATAAAAGA | 765 |
| side2_recess_5 | ATAAGAGAATATAAGGGCTTAATTGAGAATCG | 766 |
| side2_recess_6 | GCGTAACGATCATTATCATCATATGAAATAAAGAAATTGCGTA | 767 |
| side2_recess_7 | AGCCCAATAGGAAACCGGTTTAACGTCAGATGAATATACAAATCCAATCGC | 768 |
| side2_protrusion_1 | CATGTAATTCAACAGTAAGTACCGTTAACCTCCGGCTTAGGTTGG | 769 |
| side2_protrusion_2 | TAGGTCTGAGAGAATTTTAGTGTATAAAGAGAGCAAGAGAGGGTA | 770 |
| side2_protrusion_3 | CCAACGCTTAACAAAATAAGGCAGAGGCATTTTCGAGCCAGTA | 771 |
| side2_protrusion_4 | TCCAGACGACGACCATGTTCACAATTTTGAAACGATTTTTTGTT | 772 |
| side2_protrusion_5 | AACTTTTTGAGAATGATGTATCAATAGTGAATTTATCAAAATCA | 773 |
| side2_protrusion_6 | GCTGAGAACAAATATCTACCTTTACAAAAGGTAAAGTAATTCTG | 774 |
| side2_protrusion_7 | GTTATATAACTATCTGATGCAGTAACAGT | 775 |
| side3_recess_1 | GTATTCTAAGAACGCGAGGCGTTTATCCTAATTTA | 776 |
| side3_recess_2 | TCGCTATTAATCAAAATCGCGCAGAGAGGTGAGGCGGTCAGTA | 777 |
| side3_recess_3 | GCCTGCAACAGTGCCACGCTGAGACTTCGCTATTA | 778 |
| side3_recess_4 | TCTCACGGAAATGTGTTCACCTGCAGCCAGCGGTTCTATCAGAATCCTGT | 779 |
| side3_recess_5 | CGCCAGCTGGCGAAAGGGGCATACGAGCCGGTTTCGGTTTGC | 780 |
| side3_recess_6 | CGAGCATGTAGATAGTAAATCG | 781 |
| side3_recess_7 | TTGCCGCCAGCAGTCCGTGGTGAAGGGATAGC | 782 |
| side3_protrusion_1 | GATGTGCTGCCCGGTGCGATAGCTTTCCGGCACCGCTTCTGGTG | 783 |
| side3_protrusion_2 | ACTGTTGGGAAGGGCGGGCCTGCCAGCAG | 784 |
| side3_protrusion_3 | AATCCCGTAAAAAAGGCGGCCCTTTGCTTCAGACGATCCAGCGC | 785 |
| side3_protrusion_4 | GCACTCCAGCAAGGCAAAGCGCCTTGTGTACATCGACATAAAAA | 786 |
| side3_protrusion_5 | CCGGAAACCAGGCGATTAAGTGAATTTGTATGGTCATTCTTCGCG | 787 |
| side3_protrusion_6 | CAGAAACAAGACTTTCTTGGGGGGATTCGCCATTCAGGCTGCGCA | 788 |
| side3_protrusion_7 | GAGAGATGCGGCACAAGCCGATCAAACTTAAATTTCTGCTCAT | 789 |

TABLE 7
T-1 (+5°)

| | | SEQ |
|---|---|---|
| Name | Sequence | ID NO: |
| core_1 | TTAAATCAGCTCTACAGTTTCGTCCAACTTTGAAAGCGCTAATATC | 790 |
| core_2 | TAAATCAAGATTAGTTGCTATTTTTACCAGTAAGAGGGTAATTGAGA | 791 |
| core_3 | ACCTCCCGACTTGCGGGAGGTTTTAGTACCGA | 792 |
| core_4 | TTATCCTGGATATTCATTACCCAAATCTTGACAAGAACCG | 793 |
| core_5 | GCCTGAGAGTCTGGAGCAAATAGTTTGACCATTAGAAAGTTCATCAAGAGTA | 794 |
| core_6 | TCAGACTGTAGCGACACGTAATCAGTAGCGACGAGCCTAAACAAAGTC | 795 |
| core_7 | TGGCGAAAATACAAAGCGGATTGCATCAAAAAGA | 796 |
| core_8 | TAATTGCTCCTTTTTTTTGATAAGAGGTCAGATTAGA | 797 |
| core_9 | TTGATAAGCAAATATCGAACTGACACCAGTACAAACTAC | 798 |
| core_10 | TCATCGTTTTCATCGGCTGACGGAAGACAAAAGAGAGAGATCAATAGCT | 799 |
| core_11 | ATTTTCGGTCATACCATCAATATGCGTAAAACTAGCATGCCTTTAGCG | 800 |
| core_12 | TTTTCAGGGATAGCACACCCTCATAGAAAATCAAAGCCACCACCGGAACC | 801 |
| core_13 | TTGGGAAGGGCGATTTTTTGGTGCGGGCCTCTTCAGGCTG | 802 |
| core_14 | AGAACGAGTAGTAAATTGTACAACGGAGTTTTTTTTGTATCATAGGGA | 803 |
| core_15 | CGCCTGATAAATTGTGCAGTGAATAGATACATAACGCCAA | 804 |
| core_16 | AAAAGGGGCTGATAAATTAATGCCGGAGTTTTTGGGTAGCTATTTTT | 805 |
| core_17 | GTAAAGATTCAATATTTTCGAACGAGAGGCTGG | 806 |
| core_18 | CACAGACAGCCCTAACAACTTTTTATCGCCCACCAATTCTTAAA | 807 |
| core_19 | TTCGCCATTCGCTATTACGCCAGCAACGCCTGTCGCATTATAAACGTT | 808 |
| core_20 | TTCGGTTACCATAGTAAGAGCAATTTTTACTATCATAACCCTCGTTGAGAT | 809 |
| core_21 | CTGGTAAAGTTTTTTTTTTCGTCTTTCCCAGCTTGA | 810 |
| core_22 | CTGACCGAATTACGAGGTTAGCCGGAACGAGGC | 811 |
| core_23 | ATCAAAAATAAAAGAAGTATGTTAAAGTAAGCACCCTCACGGCCAG | 812 |
| core_24 | ACCCTGTAATTTTTTCTTTTGTATTTTAAATGTTTTTAATGCCTCTCAGA | 813 |
| core_25 | GAACGGTGTACAGACCAAGGGAACTTAAATTGAATTTTTGCCTCATA | 814 |
| core_26 | GGGAGAATGTTAAGCCCAACTTCTGACCTAAA | 815 |
| core_27 | CATTAGACCAAAAGGTAAGAATAAACACCGGAGCAAATCCTAAGAGCA | 816 |
| core_28 | ACCTTGCTTCTGTAAAGTGGAGCCGCCACGGGAAC | 817 |
| core_29 | CGGGAGAAGCCTTTACTGGCCTTACGCCATCAAAAATA | 818 |
| core_30 | CCAGCTTTGTCGGATTCTCTTTACCCTTTTTTACTA | 819 |
| core_31 | TGCCAAGCTTTCAGAGTCGTCGCTCGAACAAAAGAAACAA | 820 |
| core_32 | GTAACGCCTGTGCTGCAAGGCGATTTATAGTCAGAAGGGACCTGTA | 821 |
| core_33 | GACACCACGTTTACCAGCGCCAAAATTATTCACACCCTGATTTGCCAG | 822 |
| core_34 | CCACCCTCAGAACCGCATTCGCGTTTTCAACGACAATTTTTTAACCA | 823 |
| core_35 | AGTACCTTGTTAAAATTAGCATTCGCAGACGGTCAATCATAGGCGCAT | 824 |
| core_36 | ATTCAACTAATGCAAGGCTTGCCCTGACGAGAAACAC | 825 |
| core_37 | GGGCCCATGTACCGTAACACCCTTTTTAAGAAGCAAACGGAACCGCC | 826 |
| core_38 | TACATTTCGCAAATGGTTTTTCAATAACCTGTTTAGCTATTCATCA | 827 |
| core_39 | AAGACTCCTTATTACGCAACGCAAATGAAATAGAACCCACA | 828 |
| core_40 | GTTGATAATCAGAATTCAAGAGAATCGATGAACGGTAATATATTCAA | 829 |
| core_41 | ATATTTTCGAAGTTTCATTCCATATAATTTTTAGTTGATTCCCAATTCTG | 830 |
| core_42 | TGATAAATAAGGCGTTAAATAAAGTAATTCTGTCCATTTAGCGAATCCAAATAAGA | 831 |
| core_43 | TACCAGTGTTACCAGAAGGACACCAGATAGC | 832 |
| core_44 | GAGGGAAGGTTTGTCAATCATATGTACCCCGAATCTTAC | 833 |
| core_45 | TTAAAGGTAGCAGCACTGGCTTTTGATGATACAGGAG | 834 |
| core_46 | GGCGACATGGCAACATTCACCGGAACCAGGGATAAAAATTCAGTCAAA | 835 |
| core_47 | CAGGAAGATTGTAGGGGACAGATCAACGCTACTACAATT | 836 |
| core_48 | TAGAAAGCCCCAAAAACCGTTCTATGAGAAAGGCCGGAGATTTAGAAC | 837 |
| core_49 | TACCGATTTTTTGTTGCGCCGACAATTGTCGCTGAGGCTTGC | 838 |
| core_50 | TCAACCGATCAGCCCCCTTATTAGCGTTTGCCATCTTCCAGCATTGACA | 839 |
| core_51 | GAATGCGGCGGGCCGTTTTCTGAGTGAGCTAACTCAGTTGAGTGAAAGGGAGCCCC | 840 |
| core_52 | ATCGACATAAAAAAATGGTGTCCAGGAACGGTGTGCTTTC | 841 |
| core_53 | TAGGGCGCGTGCCTAAACGGTCATACCGGGGGATCAGATGCGTATAAC | 842 |
| core_54 | CCAGCTTACGGCTGGACCCGTAAAAAAAGCCGCAC | 843 |
| core_55 | ACGATTTTTTGTTAGATATAGAAGGCTTATCCGGTAT | 844 |
| core_56 | CCATCGATGAATTATCACCGTCACCGATTTTTTTGAGCCATTTGGGAATT | 845 |
| core_57 | ACCATTAGCAAGGCCGTTTTTAAACGTCACCAATGAAACATATTAT | 846 |
| core_58 | TTCCTTATCATTCCAATTACGAGCCTCAAGAGCCAGGCGGCACCAGA | 847 |
| core_59 | TGGCAAGTTGCTTTGACGATGTCACTGCGCGC | 848 |
| core_60 | CAATATTCTGAAACATCAGAATGGGACGATTGGCCTTGATAGAGCCGC | 849 |
| core_61 | GAAAGTATTAAGTCTGGCTGTCTCACTACGTAAACCGTC | 850 |
| core_62 | GATATCAATATATTTTTTTGAGTGAATAGACAAAGA | 851 |
| core_63 | CAAGTAAGCAGCCTTTACCTGAACAAGAAAAAT | 852 |
| core_64 | CCCGGAATTTGTGATTTTTAGATAGACTTTCTCGCCATGT | 853 |
| core_65 | GTCACAATCAATTTTTTGAAAATTCATATGGGAATAA | 854 |
| core_66 | ACGCGAGTTTTTAAACTTTTTCAAATATAGTTTGAAATACCG | 855 |
| core_67 | TTAATGAGTCAGAGAGAATAACATTTTTAAAAACAGGGAAGCGTTATCCCA | 856 |
| core_68 | TTTCGGAACCTATAATCGGGGTCAGTGCCTTGAGTAACAATTAAAGC | 857 |
| core_69 | AATTACCTTTTTTTTTTAGTTTTTTAATTTCATTAAAATCGCAA | 858 |
| core_70 | ACGCTCAAACAATTAACACCGCCTGCAACAGTGCCACATCTTTAGGAGC | 859 |
| core_71 | ACATTTTGCCTGAGTAAGCAAATGAAAAACCACCACCCTCATTCACAA | 860 |
| core_72 | TCGAGGCTGAGACTCATGTAGAAACAAAATTAATTACAT | 861 |
| core_73 | TTTATTTTGGGGTTTTTTCATTTGAATATCCCATCCTAATGAACGGGT | 862 |
| core_74 | ATAAGTGCCGTCAACTCAAGAAAACCAATCAATAGCGCTTAATGCG | 863 |
| core_75 | CGGCCTTGGGAAAAACGCTCATGGATTCACCACACGCTGCTTTGGGGT | 864 |
| core_76 | TCACGCAAATTAACCGTCAAACTATCTCGTTAGGGGCGCGT | 865 |
| core_77 | ACTTAAATAGAGACGCAGAAACAGCAATAATAACGGAAGTGGAGGT | 866 |
| core_78 | TAGTACCGACCCTCAGAGCAACCGAGGTTTTTAACG | 867 |
| core_79 | AACGGATTCGCCTGATTGTATCACCACCCTCAAATGAGAGGGTTGAT | 868 |
| core_80 | GGAGAAGATGATGAAACAAAGGAGGCCGATTTACTTCTTATACCAAG | 869 |
| core_81 | GCCTCCCTCATTTTTAGCCGCACCACCACCAGTTTTTGCCGCCGTTCATA | 870 |
| core_82 | CACCCTCAGAACCGCCGTACTCAATAGCCCGGAATAGG | 871 |
| core_83 | TTCAGCTAATGCAGAAAGCAAATCTAACGTCAAAAATGAA | 872 |
| core_84 | AGCCAGCGAAGAACTTGTAGCAAAAAGGGATAGAGGCGATGTGTAC | 873 |
| core_85 | ACGTACAGCCGTGGTGAAGGGATATTAACAATGCTCAGTAAAGGATTA | 874 |
| core_86 | GAGGTTGAGGGGATTAGCAGAGCCAGATTAAAC | 875 |
| core_87 | CAGGTCAAAAGCGCAGTCTCTGAATTTATTTTTCGTTCCAGTAAGCG | 876 |
| core_88 | GCTCTCACATAATACCCAAAAGAACTGGCATGAT | 877 |
| core_89 | GAAATGGAAAACAGTTAATGCCCCCTGCCTACGATGGCC | 878 |
| core_90 | ATTATTCATTTCAATTGCTTTGATGATTAGTGAGTCTAAAGCATCACCTT | 879 |
| core_91 | TGAGGCGGTCAGTAATAAATCCTCGTGCCCGTATAAAATACCGAACGA | 880 |
| core_92 | GTCACACGCGCGAACTATTTTAAAAGTTTGAGTAACA | 881 |
| core_93 | TTGTTCCAGTTTGGAACAAGAGTCAGCCTGGG | 882 |
| core_94 | AAAGAACGTGGACTCCAACGTCAAAGGATCCCACCACACCCGCCATC | 883 |
| core_95 | TATCAGGGTTTATTTTCATCGTAGGAACAAGCAAGCCGTT | 884 |
| core_96 | GTTAGATAAAACAGAGGATTGGCAGAAATACCTCCGCTACAAATCAGAG | 885 |
| core_97 | CTAAGAACGCGAGGCGTGACGACGACAATTTTTAAACAACATGACCGT | 886 |
| core_98 | GTACTGGTAATAAGTTTTAACACCAGTAGCACCATTAATCCGCACTCATCGA | 887 |
| core_99 | ACCACCAGCAGAATTAGATAGCCCTAAAACATTCAAGTTTGCGTAACC | 888 |
| core_100 | CGTTCCGACGCCAGAATCCTCGCTTTAGACA | 889 |
| core_101 | TGTGCAAATGGAAGGGAAGAAAGTTTTTGAAAGGAGCGGGCGCGGAACCCT | 890 |
| core_102 | GTGGTTTTTCTGTTTTGCCAGAGATTGCTGAAACCAGAC | 891 |
| core_103 | GATTTAGAGCTTGCGGCAAAATCCCTTATAAATCAAA | 892 |
| core_104 | ATCTGGTCCGAGATTTTCAGGTTCATCCTCATAGGTCAC | 893 |
| core_105 | GCTGAACCTCTTTTTAATATCGAGGAAGGTTATTTTTCTAAAATGCTGAG | 894 |
| core_106 | GTAGCATTAACATGATCGCGAGCTGAAAAGGTACTAACGGGAGAGGCT | 895 |
| core_107 | GAAACAATTTACAAAATCGGAGAAGTGTTTTTTTTT | 896 |
| core_108 | CAGGTCAGTTTTTGCGGATGGCTTTAAAGTACAAAATAGCAACAACAT | 897 |
| core_109 | ATATACAGTAATGGGATAACGGAACGTCTTTAAACAGTT | 898 |
| core_110 | GCAACTGCAAAAATCAGGTCCGTGAGAATGA | 899 |
| core_111 | TAAACGATCGCTGGCAGCCTCCGGGCTGGTCTCGTAAAACCCTACCAT | 900 |
| core_112 | CAGTTGAAAGAAATTGCGTAGCTGGACTTGT | 901 |
| core_113 | AGGAATTAAACCCTCAATCAATCCTTTTACCAGATGA | 902 |
| core_114 | GTTTACCACGATTATAATCGTCACCCTCAGCAAGGTGAATTACTGCGG | 903 |
| core_115 | TAAGAGGAAGCCCGAAAAACTCCAATAAATATTAGTAAAAT | 904 |
| core_116 | ATATCGCGAACAACCCCATCAACATTAAATGTTAACAGTA | 905 |
| core_117 | GTGGCACAGACAATATTTTTTTTTGAATGGCTATTAGTACTAAATC | 906 |
| core_118 | CAATATTACCGCTTTTTAGCCATTGCAACACTGGTAA | 907 |
| core_119 | CTGGAATAATGGAAGGCGCCAGGAGAACGTCAGCGTGGT | 908 |
| core_120 | CTCAACATTTCTGATTATCAGATGATGGCAATTGGGAAG | 909 |
| core_121 | ATCCAGAATATTTGCAGGTCAGCAGGGGAGAGGCGGTTTGATTGCCCT | 910 |
| core_122 | TATCATTTTGCGGAACAAAGGAAAGCGTAAGAATACAACCTGAGAGAGTTGC | 911 |
| core_123 | CTTTAATGACCAGTAATAAAAGGGACATTTTTTCTGGCCAACAGAGATAG | 912 |
| core_124 | TTCATCAATATAACCTAAACCACCAGAAGGAGCGGAATTTATTAGAC | 913 |
| core_125 | GCAACCGCAAGAAAGACGATTTTTCCAGCGCAGGCACCGGGTTA | 914 |
| core_126 | GCTAAATCGGTTGTGGCAAATCAAAATACATT | 915 |
| core_127 | GCATAAACGGAAGCAGACTTCAACAGAAAACGGGAACAAGCCTCAG | 916 |
| core_128 | CTAACAACTAATCAAAATAACCCTTCTCACCGC | 917 |
| core_129 | CCAGAGCATAACCACCGAGTAAAAGAGTCTGTCC | 918 |
| core_130 | GCAAACGCGGTCCGTTTTTTTTTTCGTCTCGTGCTGATTG | 919 |
| core_131 | ATAGATTCAATTCGACAACTCGTATTAATTTTTTCCTTTGCCCGAAC | 920 |
| core_132 | CTGGCCGTGGCGAGAAAGAATCGGCCAACGCGC | 921 |
| core_133 | GAGACCAATAAATCATATATGCAACAGAGCTTAGGGGTAATCATTGAAT | 922 |
| core_134 | CAGGCAAGGCAAAGGATTTAGAAGATCATCATATTCCTACTAATAGTA | 923 |
| core_135 | ATATAATGTGAGAATTAGCAAAATTAAGCAATAAAGCGAGTAATGTGTA | 924 |
| core_136 | ACGGCGGATTGACCGGAGCGAGTTTTTAATTAGTTACCAAAAACATTATG | 925 |
| core_137 | TTAAAGGCCGCTTTTGCGGGCCAAGCGCGAAACAAAGGGCTTGATTAGGAATACCA | 926 |
| core_138 | GAATAGCCCGAGATAGGCATTAATTGCGTTTTTTGCGCTCACTCGGCC | 927 |
| core_139 | TCAGGACGTGGTTTGCCCCAGCAGAGCAAGCGGTCCACGC | 928 |
| core_140 | GATGGTTTAATTTCAACTTTAATCACCCCCAG | 929 |
| core_141 | GCCCGCTTTCCAGTCGTCCGAAATACGGGGAAAGCCGGCG | 930 |
| core_142 | ATTACCTTATGCGATTTTAAGAACTAAAGACTTTTGCAAAAGAATTT | 931 |
| core_143 | AGACGGGCAACAGCTGCGTATTGGGGTTAGAAAGAAATAA | 932 |
| core_144 | ACCGTGCAGTTGGTGTAGATGGGCATAATCAGTGAGGCGTATCGGG | 933 |
| core_145 | GAAGATCGCACTCCAGATTTTCTGCCATAAATAATCGTCA | 934 |
| core_146 | AGACGTTAGTAAATGACCAGCTTTCCGGCACCGCT | 935 |
| core_147 | GATTATACTTCTTAGCACCAGTGAAAAATCTTATACCAG | 936 |
| core_148 | GGCGGGTGGTGCCTTTTTTCCCACGCAACCTGCAGC | 937 |
| core_149 | GGTGTCTGATTTGGGGCTACAAAGGCTATCAGGTCAT | 938 |
| core_150 | CAGCGGTTTTTTCCGGTGCCCCCTGCATTCTCTGTGGTGCTG | 939 |
| core_151 | GACGACGATGGATAGCGTCGCATAACCGATAT | 940 |
| core_152 | TGATCCTGATTGTTTGTTTACAAAAGAGCCGTCAATAGAT | 941 |
| side1_recess_1 | ATACAAATTCTGCACCCAGACGAGCGTCTTTCCAAGAATCAAGTAAATAT | 942 |
| side1_recess_2 | AGCCCAATAGGAAGGAAGGGTTTTCCCAGTCACGACGTTGCCTTTTTAACC | 943 |
| side1_recess_3 | GCGTAACGATCTGCCGGAAACCAGGCAAAGCGCCATAAGTTGG | 944 |
| side1_recess_4 | ATCAACGTAACAAAGCTGCTCATTTCGAAATCCGC | 945 |
| side1_recess_5 | GACCTGCTCCAGACCATAGTTA | 946 |
| side1_recess_6 | TCCGGCTTAGGTTGGGTTAATCTTACCGAAGCTGATAAAGGT | 947 |
| side1_recess_7 | ATCGCCATATTTAGTTTAGTATCATATGCGTT | 948 |
| side1_protrusion_1 | ATTTATCAAAATCAGAGACTATAAAACGA | 949 |
| side1_protrusion_2 | TAAAGCCAAAAAGCCTACAACGCCGCTGAGAAGAGTCAATAGTGA | 950 |
| side1_protrusion_3 | AGCGATAGCTTAGATGCTGATATCATAATAGAATTGATAACTGAA | 951 |
| side1_protrusion_4 | TATAACTAATTTCTGATAGGTTCCCTTAGAATCCTTGAAAACAT | 952 |
| side1_protrusion_5 | TACTAGAACGAAGAGTAATCTCAACAGTAGGGCTTAATTGAGA | 953 |
| side1_protrusion_6 | ATTAATTATATGTAAATTAAGACAACATGTAATTTAGGCAGAGG | 954 |
| side1_protrusion_7 | CATTTTCGAGCCAGAATATAAGAAGCCTTTACAAAATAAACAGC | 955 |
| side2_recess_1 | TACCTGAGCAAAAAAATTCTGCTCATTTGCCGCCAGCAGTTTGCTCGTCAT | 956 |
| side2_recess_2 | CAGTACATAAAACCTCACCGGAAACAATCGGCGAACGGATCAA | 957 |
| side2_recess_3 | AAACATCCCTTACACTGGTCGGGAGCTAAACACAATCACTTG | 958 |
| side2_recess_4 | GAATCATTACCGCGCCCAATAGCACGCGCCTGTTT | 959 |
| side2_recess_5 | ATCAACAATAGATAAATGGAAA | 960 |
| side2_recess_6 | GCTGTTTCCTGTGTCTTCGCGTCCGTGAGCCT | 961 |
| side2_recess_7 | CCTCACAGTTGAGGGCGAAGAACCATCACCCAAACGCCATTATTATTTAC | 962 |
| side2_protrusion_1 | CGGGTACCGTGCCTGTTGAAATTGGTATGAGCCGGGTCACTGTTG | 963 |
| side2_protrusion_2 | AGCACGCGAGCATAGGAAGCTCGAATTCGTAATCATGGTCATA | 964 |
| side2_protrusion_3 | CAACATACGAGCCAAGTGTAACACTATTCGAGGTGCCGTAAAGC | 965 |
| side2_protrusion_4 | AATCCGCCGGGCGTTAACGGCTTTCTGCCACTATGGTGTAGCGGT | 966 |
| side2_protrusion_5 | CCCTGCGGCTGGTAGGTTTCTTGGGCGGT | 967 |
| side2_protrusion_6 | GTGTTCAGGGGGTAAAATGGGTCATTGCAGGCGCTTTCGCACTC | 968 |
| side2_protrusion_7 | GCATCAGCCAAATCGCGGTTGCGTTATCCGCTCACAATTCCACA | 969 |
| side3_recess_1 | GCGAAAATCCTGTTTGATGGTGGTGGAAACCTGTC | 970 |
| side3_recess_2 | GTGCCAGCTGCATCTGCCAACG | 971 |
| side3_recess_3 | TCTGCCAGTTTGAGGGGACGACGAAAAAAGGCTCC | 972 |
| side3_recess_4 | GCAGCACCGTCGCCTTTAGTGATGAAGGGTAAAGTGCATCGTA | 973 |
| side3_recess_5 | ACGTAATGCCACTGGTAGCAACGGCTACAGAG | 974 |
| side3_recess_6 | AAAAGGAGCCTTTAATTGTCCCCCTCAAATGGCCTCAAAGCG | 975 |
| side3_recess_7 | GCTTTGAGGACTGGCTCATACGTTAATAAAACGAGGCATCAAGTTTTAAA | 976 |
| side3_protrusion_1 | TTTTCATGGGAACGAGACGAAGGCACAACTAAAGGAATTGCGAAT | 977 |
| side3_protrusion_2 | CAGCATCAGGCACTTAAAAAAGTTTCCATTAAACGGGTAAAAT | 978 |
| side3_protrusion_3 | CAAAAGAATACACCATCTTTGATTGTGATATTACAGGTAGAAAG | 979 |
| side3_protrusion_4 | AATAATTTTTTCATCTCCAAACAGTATCG | 980 |
| side3_protrusion_5 | TATGGGATATCAGCTAGAAAGGAACCAACCTAAAACGAAAGAGG | 981 |
| side3_protrusion_6 | GCGGAGTGAGAATTGCTTTCGGCGAAAGAGTTTAGACTAAAAACC | 982 |
| side3_protrusion_7 | ATCGGTTTTTTAAAACGTTGGCTAAACAACTTTCAACAGTTTCA | 983 |

TABLE 8
T = 3

| | | SEQ ID |
|---|---|---|
| Name | Sequence | NO: |
| core_1 | GCAGAGGCATAACGGAAACTACAATTAGCGTAATAAACACCAGAAT | 984 |
| core_2 | CAGTTGAGCCTGTCGTAGAGAGTTTACGAGCCCCGTGGTGGTTCCGAA | 985 |
| core_3 | GAGCTTGACGATGGCCTTTGGGGTCGAGGTGGGAAGCATACAATTCC | 986 |
| core_4 | CGCGCTTAATGCGCCGCTACGAATCAGAATCGGAACATCCGCTC | 987 |
| core_5 | CGTTTTCAGACAGCTACAGTAACAGTAGAATTGTTTAA | 988 |
| core_6 | TAACGAGCGTTAGATTTTGCACCTTTTTAGCTACAGAACGCGA | 989 |
| core_7 | GATGAATACCTCATAGCGCCTGTAGCATTCCACCTCATTTCATTTCAA | 990 |
| core_8 | GAGATAAAGAAATTGACGTATAACACTGAGTACATGAAA | 991 |
| core_9 | CGTCTTTTAGAAAATTATTTGCAGCATTAGAAGAATAACGATAACCC | 992 |
| core_10 | TTCGCCTGTTCAGGTTTTACAGAGCGGCAATAATAAGAGCTCACGGAA | 993 |
| core_11 | GTGAGAATGTCATACATGGTTTTTTTTTGATGATACAGGAGTG | 994 |
| core_12 | CCGATTTACTTCTGAAATTTCTGCTCATTTGCCGCCAGC | 995 |
| core_13 | AAAATCGCGCAAGCAAATCAGTAAATTACCGCGCCCAATAGAAACCAA | 996 |
| core_14 | TTCTCCGTGGTGAAGGGCAAATGAATTTTCTGTTCAGCGG | 997 |
| core_15 | ATCAAGAAGAATTACCTTTTTTAATGGAAACAACAAGAAAGCAAAAGA | 998 |
| core_16 | AATCCTGACAGATGATGGCAATTCAACAACTTTACTGGTAACGTTTGT | 999 |
| core_17 | CCAAGCTTTCAGAGGTGGAGCCGCCATTTTTGGGAACGGATA | 1000 |
| core_18 | ATATAAGTGGATAAGTGCCGTCGACAATACTTTATCGGCCTAATCAGT | 1001 |
| core_19 | GGAAACAATCGGCGAAGATAGCTCAAGAAACACGGAATTT | 1002 |
| core_20 | CAAGTTACTGCGGCCAGCGCGCCTGTGCACTCTTTAACAAAGCATGTA | 1003 |
| core_21 | ATAAGTTTTAACGTTGACGCTCAACTGATTATTTGTTTGGATTTGGCAA | 1004 |
| core_22 | AGAGCCACCACCATCGGCATTTTTTTTCGGT | 1005 |
| core_23 | TAATGGAAGGGTTAGATCAATATTTGCTTTG | 1006 |
| core_24 | AGGGCGAAAAACCGTCAGTTGGGCAAGGAGCGGGCGCTAGGGCGCATA | 1007 |
| core_25 | TATCAGGGGGGGGAAAAGCACTAAGCGGGAGCAGACAGGA | 1008 |
| core_26 | ATTGGGTCAGTGCCTTGAGTAACATATTTCGGTTCGTCAACCCATG | 1009 |
| core_27 | GCAATAGCTATCTTACACCTCACCTGAATTTTTTTATCACCGTCACC | 1010 |
| core_28 | CGAAGCCCTTTTTATTTTTGAAAAGTAAGCAGATAGCCGA | 1011 |
| core_29 | AACAAAATTAATTACATGTATCACAGCAAGCCTAGGATTA | 1012 |
| core_30 | TCAACAGTTATGGGATTTTGCTAAACCTACCATATCACTAAAGAGAC | 1013 |
| core_31 | TCATTAAAGGCCGCCGCCAGCTTTTTTTGACAGGAGGT | 1014 |
| core_32 | GTTAATGCCCCCTGCCGTGCCCGTACGATCTAAAGTAGCCGTAGATT | 1015 |
| core_33 | GTATTCTGGTAATATCATACCTACCCTCGTTAAGGGCGCG | 1016 |
| core_34 | CAGAACAAACGGTACGGAGGCCACGCTCGAATGTCATACCCCCTGCAT | 1017 |
| core_35 | AATAAGAACCGACTTGCGGGAGGTAATAGATAAGTCCTGA | 1018 |
| core_36 | GAATGCGGGGGGCCGGAGGGTTGTGTTTTTATTGAAGAGGCTGAGAC | 1019 |
| core_37 | CTTCTGTAACAGAACGCGCCTGTTTTTTTATCAAC | 1020 |
| core_38 | AAAGTGTCTGCCCGCTTTCCAGCGGTGCCGGGGGTTTCTGCCAGC | 1021 |
| core_39 | TCCTCAAGTACCAGGCATAGCCCGGAATAGGTGTGGTGC | 1022 |
| core_40 | TAGAAGAACTCAAACCTTTGATTAAATTAACCGTTGTAG | 1023 |
| core_41 | AATGAGTGGAAGTTTCCTGTGTGCTGAGAAGCGTGCTTT | 1024 |
| core_42 | TTCTAAGAGAGAAACAGAATTATTTCAGGGATCGTACTCA | 1025 |
| core_43 | TTTGATTTTTAGCCTTAAATCATTATTTATTTTTCCCAATCCA | 1026 |
| core_44 | CACCCTTTTTTAGAACCGCCACCCACTATATGTATTTTAGAATAAACA | 1027 |
| core_45 | GGGAATTTTTTAGAGCCAACGTACAGCGCCAAGG | 1028 |
| core_46 | GCAGAAACAGCGGATCAGGGAAGCCGTAAAACAGAACCCTACTATGG | 1029 |
| core_47 | AGAACCACGGAGGTTTAGTACCGCAAACAAACTTACCTGA | 1030 |
| core_48 | CACCAGAGCGGGGTTTTGCTCAGAGAAGGATCAATAGGACCAGTACA | 1031 |
| core_49 | AATAATATCGTTTTAGCGAACCTCACGATTTTTCTTACCAACG | 1032 |
| core_50 | TTATTCATTAATGTTTACCAGTCCATGAAATACGTCAAAAATGAAAAT | 1033 |
| core_51 | ATCGTCGCGATGCAAATCCTTTTTATCGCAAGACAAAGATGATG | 1034 |
| core_52 | AGCGGTGCTCGGGAAAGATCCCCGACACAACAGCAGCAAGCGG | 1035 |
| core_53 | CAGACGATCCAGCGCAAATTGCGTATGGTCATAGCTTACTACCGTAA | 1036 |
| core_54 | GTACATAAATCAATATATGTGAGTGATTTTTTAACCTTG | 1037 |
| core_55 | CAAGTACCGCACTCATTAGGAATCCTCACATTGTGTCACT | 1038 |
| core_56 | AGAACGGGATCCGGTAAGCAGCCTTAACGTCAGTGAGAGA | 1039 |
| core_57 | TCCTCACTGTTCTTTTTTTGCGTCCGTGAGCCGGGTCACTGT | 1040 |
| core_58 | GGGCGCGGGGTTTGCGAGTGAGACGGGCAACAAAAAGAAT | 1041 |
| core_59 | CGCATCGGACAGTATCGGCCTCAGGAAGATAATATTC | 1042 |
| core_60 | GTTCAGCAAATCGTTAACTTTTTGCATCAGATGCCGGGTTACCTGCAGCC | 1043 |
| core_61 | CATAAACATCCCTTACACTGGTGTGGAGAGGCTTGCGGTATGAGCC | 1044 |
| core_62 | TTGCTCGTGGCTGGTAATGGGTAAAAATATCAATCTTTAG | 1045 |
| core_63 | TCCATGTTATAAGGGAACCGAACAAAAATCTOCCAATTC | 1046 |
| core_64 | GTGTAGCGGTTTTTCACGCTGCGTTTTTTCGTCTCGGGG | 1047 |
| core_65 | TCAGCGGGGGTCAATCACTTAGCCGGAACGAGAGGTTTCT | 1048 |
| core_66 | GACAATATCTGGCCAAAAGAATACAATAGATACAACTA | 1049 |
| core_67 | TTGCCCGCAATAGATTAAAAGCATCGAGCCAGCAGCAAATGGTTTAGCT | 1050 |
| core_68 | CCGCCTGCAACAGTGCGAAGATAAGAGCACTAAATACATTTTACGGCT | 1051 |
| core_69 | GCCAGCTGCATTAATGATTTTTTCGGCCAACGCGCGG | 1052 |
| core_70 | ACGGCGGATTGACCGTAGAAACCACACCAGTCAGGCCGAACGTTATT | 1053 |
| core_71 | ATCAATATCTGGTCAGTTGGTTTTTAAATCACGCGTGC | 1054 |
| core_72 | CGATTAATTTTTGGGATTTTTAAACATTTTTGAGGCTACAAACA | 1055 |
| core_73 | TAGAAGTACCATTGCTGAGGCGGTCCCTGA | 1056 |
| core_74 | GGTACCGACGAGTAAAAGAGTAGTTGATTTTTAGGAATTGAG | 1057 |
| core_75 | TAGTAGCATGCGAACGCATATAACCCAGAACG | 1058 |
| core_76 | TTAATACCGAACGAACCACCAGCACACGCTGAACCTTGCTGAACCTC | 1059 |
| core_77 | AACAGAGGTCTGACCTGAAAGCGTCAGAGATATAAATCCTTTGCTCA | 1060 |
| core_78 | AGTAGATTCTGCTCATTTTGAAAGATTGTGTCGAAATCCGCGACCTGC | 1061 |
| core_79 | AGCCAGCTTTCCGCAATAACCAGACGACGATAAAAA | 1062 |
| core_80 | CAGGTAGAAAGATTCATCAGTGAATTACCTTATGCGAAACAAAG | 1063 |
| core_81 | AGGACAGATGAACGGTAGCAGATACATTTCGCATTTGGGGC | 1064 |
| core_82 | ATCGCCATTAAAAACACTGATAGCGGCTATTAGTCTTTAA | 1065 |
| core_83 | GCGCAGACGTCATTGCAGGCGCTTCAACCAGCTGAGGATTACTCGTAT | 1066 |
| core_84 | TATTACTTTTTGCCAGTTAGACTTGAAGGTTAAATCCGCCTGCCCTGC | 1067 |
| core_85 | CTTATAAATCGCTGATTGCCCTTTTTTTACCGCCTGGCCCTG | 1068 |
| core_86 | TGGTCAGCAGCAACCGAGCACATCAATTTTAAAAGTTAACCACCACACCCG | 1069 |
| core_87 | CCTCGTTTCTGCGGAATCGTCATACGCACTCCGCCCGAAA | 1070 |
| core_88 | TGCTGGTCGGAGGTGTCCATGACGAGAAACAAGTTGATTGATGGCTT | 1071 |
| core_89 | CAGGCGCATACAACGGAGATTTGTATCATCGCCTGATAA | 1072 |
| core_90 | TGCGCGAATCCAATAAGAGCATAAAGAGCTTACCTTTAATCATAAAT | 1073 |
| core_91 | AAAACGAGAATACGTCAGCGTGGTTTTTGCGGAGCCGTC | 1074 |
| core_92 | AGCTAAAAGGGACATTTTTTGAATCCTAAAACGTGGCACA | 1075 |
| core_93 | GAACCCTTTACATTGGCAGATTCCAGAAGGCATTTTGOGGAACAA | 1076 |
| core_94 | CCAACGGCAGCACCCCAGCCCGAGGAGTCCACTATTAAAG | 1077 |
| core_95 | AAAAAAGCCGCACAGGCGGCCTTTAGTGATGACGGCAAACGCGGTCCG | 1078 |
| core_96 | AGGTCTTTAGTAAGAGATATAATGTCTGGAAGTTTCATTC | 1079 |
| core_97 | AAATGGTCAATAACCTTGACCAACTCAGTGAATAAGGC | 1080 |
| core_98 | TCGTCTGAATTTTTTGGATTATAACAGGAAAATTTTTCGCTCATGGAA | 1081 |
| core_99 | AGAGAGTAATTGCTGACAGTTCAGAGTAGTAAATTGGGCTTAACAAAG | 1082 |
| core_100 | CTCATAACTGCCGTTCAGGGTAAAGTTAAACGAGTTTGAGGGGACGAC | 1083 |
| core_101 | AACCTGTCCATCACGCAGTAATAACATTTTTCACTTGAGT | 1084 |
| core_102 | TGGGAACAATTGGTGTAGATGCGTTTTAATTCG | 1085 |
| core_103 | TAAAAAAAACGTGGACTTTTTCCAACGTCAAATCGGCAAAATC | 1086 |
| core_104 | ATCCCGTAAAGAATTTTTAGCGAGGTTGTGTACATCGAC | 1087 |
| core_105 | CGTTGAGTAACATTATAGCGGAATTATCATTTTTCATATTC | 1088 |
| core_106 | ATGCTGATGGAACGTGCCGGACTTGTAGAGACTGCTCCTTAGGTCACG | 1089 |
| core_107 | TAACCGTGCATCTGCCAATGGGATTTGATAAGACACGACC | 1090 |
| core_108 | CAGCAGGCTTTTTAAAATCCTGTTTGATAAGCCGGCGAACGTGGC | 1091 |
| core_109 | ATAATTACCTTTCCAGAGCCTAATAGGGAAGGTAAATATTGACGGAA | 1092 |
| core_110 | ACCCTGACTATTATAGTTTTTTAGAAGCTACATAACGTTTTTCAAA | 1093 |
| core_111 | TAGTTTGACCATTATGTCAATTTTTCATATGTACCCCGGTTG | 1094 |
| core_112 | ACCGAGCACAAATATTTCTACAAAGAGAGGGTAGCTATTTCCCTCAGA | 1095 |
| core_113 | CCAGACGTTAGTAAAATCACCAGTAGTTTTTACCATTACC | 1096 |
| core_114 | GGGCGACAGGAGCCTTCAGTCACGACGTTGTAAAACGAC | 1097 |
| core_115 | TAAAGACTTTTTCATGGGCTTGCACAACTATTTTTAGTACGGTG | 1098 |
| core_116 | CCACCTGTAGCCAGCTCCCGTCGGATTCTCC | 1099 |
| core_117 | AGAAAGGACACGTTGAAAATCTCCAAAAATTTATTAGCAA | 1100 |
| core_118 | AGTAATAAATCGGTTGTACCATTTTTAAACATTATGAAAATTAAGCAAT | 1101 |
| core_119 | GAGTAATGAAACGTTGTATAAGTCGGAACGAATCATAGAAGAGTCA | 1102 |
| core_120 | CAAAATAAGACTTTTTTGGATAGCGTCGCACCGC | 1103 |
| core_121 | GAAACCGAATTGAGGGTCATATGGTTTAGCGTCCTTATTAAAATAAATC | 1104 |
| core_122 | GCTTCAAAGCGAAGCTGCGCAACTGTCATGCCATTCG | 1105 |
| core_123 | AAACGAACAATGCAGAGTAATCTTGACAAGATTTTTCCGGATATTC | 1106 |
| core_124 | GGCCGGAAAGTTTGCCTTTACCAGTAAATAAGTATACAA | 1107 |
| core_125 | ATAGTGAAACGAAGGCACCAACCTAATACGTAATGCCACT | 1108 |
| core_126 | TGCGGGAGGCATCAAAAATAATTGCTCGAGGTGAATTTCTTAAAC | 1109 |
| core_127 | CAAAATAAACAGCCATAAGATTAGTTGCTAAAACATGTTCAGCTAATG | 1110 |
| core_128 | ACCAGAAGGGCCAGTGGACTTGAGCCAAAAGGCTCCAAAATTCAACCG | 1111 |
| core_129 | GGGCGATCGGTGCGGGCCCACGCGCTCATTTTCGCATTAAATTAGC | 1112 |
| core_130 | AGCTTGAAAAATGAAACAATTTTTTACAACTAAAGGAATTGTGTGAGCG | 1113 |
| core_131 | AATACTTTAGTAACAATTCATCAACATTAAAATCAGC | 1114 |
| core_132 | CGCGTCTGGCCTTGACCGGAAGCATTAAATCAATAACCGA | 1115 |
| core_133 | AAGCCTCAATCATACAGGCTTTTTAGGCAAAGAATTTTTGATAATCAG | 1116 |
| core_134 | AAGCCTGTTTAGTATCGAAAATTTGCCAGTTAACAAAGTT | 1117 |
| core_135 | GAAAGCGCAGTCTCTCACAAACGCGTTTGCAGCCACCAACCTAAAT | 1118 |
| core_136 | ACGACGACTTAATTTCCCGGAATCCATAGCCCCAGACTGTAGCG | 1119 |
| core_137 | TTCAACCGTTCTAGCTGATAAATGAGACAGTGGAAGATT | 1120 |
| core_138 | CTGTAGTTTTTTCAACATGTTTAACTCCAATTTTTAGGTCAGGATT | 1121 |
| core_139 | CATCAATATGGGTGGCATTTTTTAATTCTACTAATAGATATTTTC | 1122 |
| core_140 | CATAATCAAGTAGCGACCGAACGCAAGGATAAAAATTTTT | 1123 |
| core_141 | TGTAGGTAAAGATGGCCTTGATATTGAATTTACAGAATCAACGTCACC | 1124 |
| core_142 | AATATTTTCCCTCAGCAATGACAACAACCATCGCCTCTTC | 1125 |
| core_143 | TAAATATGGGGAGTTATATATTCGGTCGCTGAAAAGCGGATTGTGGGAA | 1126 |
| core_144 | CGAGCTGAAAAATACAAACAAGAGAATCGATCCATTAA | 1127 |
| core_145 | TGAGGCAGGCCACCACACCCTCAGAACCGCCATTTTTAACTTAATGGT | 1128 |
| core_146 | GTCAGACGGGAACCAGCATCTTTTTAAGGCGTCGCCAAAG | 1129 |
| core_147 | AAATCACCATTTCAAAAGGGTGAGAAAGGCCGTAATGCCG | 1130 |
| core_148 | CGAATAATCATCGATAAAGCCTTTATTTCTTCCAGTAAGC | 1131 |
| core_149 | TCTGGTGCCGGAAACCATAATAGTAAAATGTTTGCGAGAGGCTTTTGCA | 1132 |
| core_150 | CCATTCAGGGGGGATGTGCTGCAAACGCCAGCTGGCGAAA | 1133 |
| core_151 | ATAGGAACGCCAGCAGCGCCGACAGCGAAAGACACCGTGTGATAAA | 1134 |
| core_152 | GATCGTCAGTTAAAATTTTAACCACATATATACCCTGT | 1135 |
| core_153 | CCACATTTTTTTCAACTTAACGGAAACCAGTCAGGACGTTGACTAAAAC | 1136 |
| core_154 | GCCTGAGACAAAAACACAAATCACTTTAAATGCAATGCCTAGAACCCT | 1137 |
| core_155 | ATCTTCTGCCGGAACCGCCTCGAACCGCCACCCT | 1138 |
| core_156 | AAAACTTTTTCAAATATAAATGCTTATTAATTAATTTTCCCTTAGAAT | 1139 |
| core_157 | TACCAAGCGCGATTTTAAGTTTTTACTGGCTCATTATCAACATTATT | 1140 |
| core_158 | GGCTATCAGGTCATTTTTATCAAAGGGTAGCAACGGCTA | 1141 |
| core_159 | CTCCGGCTAACATAGCGATAGCTTAGATTAAGTTAATTGATTGAAAT | 1142 |
| core_160 | TAGGCTTTTTGGCTGACCTTACTCATCTTTGACCCCCAGCGATT | 1143 |
| core_161 | GTTATATATCACCTCAGAGCCGCCCCTCAGAGCCGCCAC | 1144 |
| core_162 | CATCAAGAAGGAAGTTTGAACGGTAATCTTTTTTAAAACT | 1145 |
| core_163 | AAGGCCGCTTTTGCGGCAGAGGCTAAAAGCCCGTCTGGAG | 1146 |
| side1_protrusion_1 | GTAATTGAGCGCTAATTGAACAAATGAACCATAAACTTAA | 1147 |
| side1_protrusion_2 | ATTGCTTTAGCATATAGAAGGCTTTATTAAACACAAGAATTGA | 1148 |
| side1_protrusion_3 | TTTTCACGTCGTAATCTGCGCTCAAAAGCCTGAGCAAGCC | 1149 |
| side1_protrusion_4 | GTTTTTATTTTCATCGCGAGAACAGGGTGCCTCACCCAAA | 1150 |
| side1_protrusion_5 | TTTCCTTATCATTCCATCAATAATAATTTACGTTTCATTT | 1151 |
| side1_protrusion_6 | CCAGAATCAAATTGTTCCTAAAGGTCAAGTTTCACTACGGTCAGAGG | 1152 |
| side1_protrusion_7 | GTTAAGCCGAGAATTAACTGAACACCCATCAGAGAATAAAAAC | 1153 |
| side1_protrusion_8 | AATTTTATCCTCCTTTTACATCGGACGCGAGGCCCATCCTCGGCTGTC | 1154 |
| side2_recess_1 | AGAAAGGAATCGCTGGCAGCCTTGGAACAAATAGGGTTGAGTGTTG | 1155 |
| side2_recess_2 | CAAATGCTTTAAACAACACTATCATAACCGAGGCAT | 1156 |
| side2_recess_3 | GGTTTTTCATCCCACGTCGCACTCTCTAAAATAACCCTCAATTAACA | 1157 |
| side2_recess_4 | CCACGCTGGTTTGCGTCGGTGGTGCCTTTTCACCTATTGGGCGCCAGGGT | 1158 |
| side2_recess_5 | GTACAGACATTACCCAATCATTGTTGAGATTTTTAATTT | 1159 |
| side2_recess_6 | TTCCAGTTCCGGCCAGCAAGAATGATTCGACA | 1160 |
| side2_recess_7 | GACTTCAAATATCGGGCAAAAATCATTGAATCCCCCT | 1161 |
| side2_recess_8 | CAACTTTAAATCAACGTGAGATGGTAGGAATAAGGAATTA | 1162 |
| side3_recess_1 | TTGAGGACACGGGTAAAAAACGAAAGAGGCAAAAGAATACGGAAGAAAAAT | 1163 |
| side3_recess_2 | TTGCTTTATTGGCGATTAAGTTGGGTAACGCCAGATAAAAGAAAC | 1164 |
| side3_recess_3 | GCCAGAGGGGGGGCAAAGCCAAAAAGATTATTTTTGAGGAA | 1165 |
| side3_recess_4 | AACAGTAGGGCACGCTGAGGTCTGAGAGACTACCTTGAGAGA | 1166 |
| side3_recess_5 | GCAAAGACACCACGGAATAACAATACCGATAGTTCCGTAATCTAAATTGT | 1167 |
| side3_recess_6 | CTACGTTAATAAAAGAAGTTTT | 1168 |
| side3_recess_7 | TAACAACGCCAACCAGTATAAAGCCAACGCTC | 1169 |
| side3_protrusion_1 | CAGTAATAAGAGACTGTCCAGCCTTGAATAGGTTGG | 1170 |
| side3_protrusion_2 | TTACGCAGTATGTGGCAACATGGTTTTCCTAATTGTATCGGTTT | 1171 |
| side3_protrusion_3 | AGTTTATTTAAAGGTTAGCAAACGTAGAAAAGGAAACGCAATAA | 1172 |
| side3_protrusion_4 | AGTAATTATATAAAGTACCGACAGAATCGCCATATT | 1173 |
| side3_protrusion_5 | AAAGGTAAATTCTTACATGTAATTTGGCATGATTAAGACTCCTTA | 1174 |
| side3_protrusion_6 | TAACGGAATACCCAATCAATAATATGCGTAATAAACA | 1175 |
| side3_protrusion_7 | TACATACATTGTCACAAAAGAACTAGGCAGAGGCATTTTCGAGC | 1176 |

TABLE 9
T = 4 (iso)

| | (iso) | SEQ ID |
|---|---|---|
| Name | Sequence | NO: |
| core_1 | CAAAAACCTGTCGTGCTTTTCTTTTTCACCGCATTGGGCGC | 1177 |
| core_2 | ATGGTCGCTCACTGCCCGAACGTGGGGAACAAAGCAAACTAGTATGTT | 1178 |
| core_3 | TGTACTATTATAGTCGTACCAGGTATTAAAGCTTTCCAGTCGGGACTA | 1179 |
| core_4 | CGGCATTTTTTAGATGCCGGGTACATCCCTTA | 1180 |
| core_5 | TAGGGTTGAAATCCTGTGCCAGCCGCGCGGGGAGAGGCGTTAATGAA | 1181 |
| core_6 | TTTGATGAAATATTGAGTCACCGTTAGCAAGGCCGGAAGAGGAAA | 1182 |
| core_7 | GAATTCGACGCGTGCCTTTTTGTTCTTCGCGTCCGTGA | 1183 |
| core_8 | GCCTATTGACGTTTTAACCCTCATTATACCGTTCCAGTAAGTTATCACC | 1184 |
| core_9 | TAGCCGGACGCCTGATCGGGGTTTGGTGCCTAATGAGTAAATGAATTT | 1185 |
| core_10 | ATTAATTGCGTTGCATGGGCGATGGCCCTCAAGAAGCAA | 1186 |
| core_11 | CGGAGATTTGTATCATCACTAAATAAGCCTGGTGCACTACGTGAACC | 1187 |
| core_12 | CAACGCCCCAGACGTTGCCGTCGGAAGGATTGGTAATAACTGCTCCA | 1188 |
| core_13 | CGAGAAACACCAGAACGAGTAGTAAATTGGGCCCAAATAAGAAACGAT | 1189 |
| core_14 | CTGGCCCTGAGAGAGTGATTGCCCTCACCAGTGAGACGGG | 1190 |
| core_15 | AGCTTAATTGCTGAATGCAGGCGAAGTGTTGTTCGGCCAATAATC | 1191 |
| core_16 | AGGGGGTAATAGTAAATGAGAGATGAAAACGAAAGATTAA | 1192 |
| core_17 | AAAGAAGTACGCATTTTTCCAGCTTACGGACGACGATAAAAAC | 1193 |
| core_18 | ACCCGATTGAGGGAGCCGCCACCCGGATAAGTAGTGAGCTAACTCAC | 1194 |
| core_19 | CGGAAATTTTTACCAGGCCGCGGGATCGTCAC | 1195 |
| core_20 | CAAAATAGCGATTTTTAGGCTTTTGCAAGCTCTCACGGAAA | 1196 |
| core_21 | CTAAAGGTTTTTAGCCCCACATACGAGTTTTTCGGAAGCATAAAGTGTA | 1197 |
| core_22 | CTTTCCGGCATTTTTCGCTTAGCAATATTTTTAGCCTCAGAGCA | 1198 |
| core_23 | GAATGCCAACGGCAGCACCGTCTTTTTGTGGTGCCATTTTAAACAGTTCA | 1199 |
| core_24 | GCATCAAAGAATGACCATCACCCAAATCATTGCAGGCGCT | 1200 |
| core_25 | GTTGTAAAGTCTTTACCCTGACTAGGATTAGAAATTGTG | 1201 |
| core_26 | GAATTTGATGTTTAGTAAATATTCATTGAATTCAAGTTTTTTGGGGT | 1202 |
| core_27 | AGACTTTCCTGCAAGGCGATTAAGCGTTGGTGCGCGTTTTAGAGAGTA | 1203 |
| core_28 | CGCCAAAGTAAAGGTGCGCAATAATAACGGAATACCCAAA | 1204 |
| core_29 | TCGCACTGCATCTTTTTAGCGGGGCCCCCTCAAATGCCCCTTTGCCAG | 1205 |
| core_30 | GTACGGTGTCTGGAAGTTTCATTTTTTTCATATAACAGTTGA | 1206 |
| core_31 | CAGGGTGGTCAGCTGCAGTTTGCGTCGCTGGTTTGCCCCA | 1207 |
| core_32 | CAATCATAAGGGAACCGAACTGAAAATAAATGGGGTCAG | 1208 |
| core_33 | CCGTGGGATGTTAAATAAAAATAAATAGGTCATTGGGTAA | 1209 |
| core_34 | GTACCAAACTGCGCGCCTGTGCTTATAAATCTTTTTAAAGAATA | 1210 |
| core_35 | TTTTCCCAGCGAAACGTGGCCTTCCTGTAGCCCAACCCGT | 1211 |
| core_36 | CGACGACAGGCCTCTTTTTTTGCTATTACGCCAGCTGATCTGCCAGTTTGAGGGG | 1212 |
| core_37 | GCAAAATTAAGGTCATTTTTGCGGATGCTCCTTTTAATATT | 1213 |
| core_38 | TGGCTTAGACATGTTTTAAATATGCAACTAAATGTAAACGTTGATAAG | 1214 |
| core_39 | ATAAATCAAATCGTCAACTGGATAGCGTCCAAACGAGGCG | 1215 |
| core_40 | TATTACGCCCAACAGGACAAACGGCGGATGCACGGATTCT | 1216 |
| core_41 | CAGAAACCGTCTATCAAGCTGTTTCCTGTTTTTGTGAAATTGT | 1217 |
| core_42 | CGGAACCCGAGGTGCCGTAAAGTACTGCGGAAAATCAGACGACGGC | 1218 |
| core_43 | ACTGGTGTGTTGAGGAAGCCCGAAAACGGGATGTGTCCGTGGTGAAGGGAT | 1219 |
| core_44 | CATCTTTTAAGGTGAACGTCCCTCAGAACCGCCTTGATATTCACAAAC | 1220 |
| core_45 | CGCATCGTAACCGTGCGCGAAAGGCAGGCAAAGTTTTTGCCATTCG | 1221 |
| core_46 | AAAACAGGAAGATTTTTTGTATAAGCAAATATTTAAAT | 1222 |
| core_47 | TCCAATAATCCCACTCTGTGGTGCTCATACCGGGTTTTTGTTTC | 1223 |
| core_48 | TTTGATGGTGGTTCCGAATTTTTTCGGCAAAAATCATACATAAAGCTA | 1224 |
| core_49 | GTCAAAGGGCGAACAAATCGTTAAGCCCGAGACCTTTAAT | 1225 |
| core_50 | CAACAGCTTCATTTTCCACCAGTACGCCACCCGTTTAGTA | 1226 |
| core_51 | ACGTCACCTCATATGGATTCATTACATAATCA | 1227 |
| core_52 | TCCAGTTTACTCCAACCTTCAAAGAGCGGATTCGCCAGGGCAGGTCCCG | 1228 |
| core_53 | AAATCACCAGAACTGGAGCAAACGTAGAAAATGCAGCAACTCAGAAC | 1229 |
| core_54 | AGTAGCACGCGACATTCAACGGAGAGTCCACGCGGTCCA | 1230 |
| core_55 | CATGATTAAGACTCCTATAATGCTTAAATTTTTCAGGATTAATTCGAG | 1231 |
| core_56 | AATGCTGATGCAAATTGTATCGGTTTATC | 1232 |
| core_57 | AGTAAGCATTTTTATAGCCGAACAAAGTTACCAGAAGGAAACC | 1233 |
| core_58 | AATAGCAAAATAATAAGAGCAAGAACCGATATGAACGAGG | 1234 |
| core_59 | AAACGCTCATGGAAATGATTATTTACATTGGCGACAATGAAGACGCT | 1235 |
| core_60 | ATATCCAGAACAATATTACCGCCAACGACCAGTAATAAAA | 1236 |
| core_61 | ACATGGCTGATATAAGTATTTTTTGCCCATACGTAAAAGCCACCGGAACCGC | 1237 |
| core_62 | GCGACAGATGTCACAATCTTTTTATAGAAAATAATGAAAC | 1238 |
| core_63 | TAGCGTTTGCCATCGATAGCAGCTTTTTCCGTAATCAGTATAAGAAA | 1239 |
| core_64 | GGAGCACTCGGTCAGTATTAACATCGGCCTTGCTGGTA | 1240 |
| core_65 | CCTCAGAGCTCCCTTTTTTAGAGCCGCCACATTGCCTTGAGTAA | 1241 |
| core_66 | CCGCCACCCAGGAGGTTGAGGCAGGGTGTACAGACCAGGC | 1242 |
| core_67 | GCCTTTACACCCTGAACAAATAGCCCTAAAACTTAGATTATCAATAT | 1243 |
| core_68 | GCGAAAGATAACCCACAAGAATTTATCAAGTTTCGGCATTTTCGGTCA | 1244 |
| core_69 | CCAAAAGGAGCCTTTAATTGCGAATAAACAGCTTG | 1245 |
| core_70 | AACGTTATTACCATATCAAAATTATAATCATTTTACAAAA | 1246 |
| core_71 | GGCTACACAGAGGGTCTGAAATGACCTACATTTTGACGCAACAATGA | 1247 |
| core_72 | AGCTTGCTTTCGAGGTGAATTTCTAAAAGGCT | 1248 |
| core_73 | GATTGGCCACCCTCATTTTTAGCCACCACTAGCCCCCTTA | 1249 |
| core_74 | TCAATAGTTGGCTATTCCAGTCACGCCATTGCAACAGGAA | 1250 |
| core_75 | TAAAGTTTTTTTTTTCGTCTTTTGTAGCATTCTTTTTACAGACAGCC | 1251 |
| core_76 | GAACCCAAAAATCTCCAAAATAATAATTTTTGTAGCAACTTTCATGA | 1252 |
| core_77 | CAATAGGCTGAGGCTTGCAGGGAGTTTTTTAAAGGCCGCTTTTACCCAAGCC | 1253 |
| core_78 | CCAACTTTGAAAGAGGACAGTTTTTTGAACGTCAGAC | 1254 |
| core_79 | TATATGTAGAGAAGAGAGCGATAGCATCGCCAAGGTT | 1255 |
| core_80 | CAGTTACATTTCATTTCAGTGGTTTGAAATACCGACCGTGTG | 1256 |
| core_81 | TCACGTTGCTCATAGTTAGAGGAACAACTAAAAAGACTT | 1257 |
| core_82 | TGTACCGTAACTTTTTCTGAGTTTCGTAGGGATAGCTCAGAGC | 1258 |
| core_83 | GCATAGGCATAAGGCTTGCCCTGAGCTGCTCATTCAGTGACATGCAAAA | 1259 |
| core_84 | GCAACATATAAAAGAAACGCAAAGACATTTTTCACGGAAT | 1260 |
| core_85 | CATCGCCCACGCATATCAATCGTAATTGAGCGCTAATAT | 1261 |
| core_86 | CAGCGATTCTAAAACACTCATCTTTTTCAACGTAACAAA | 1262 |
| core_87 | TTTAACAAAAATAAACCCAGAGCCTAATTTGCTTGACCCCAACAGTTT | 1263 |
| core_88 | GACAAAGAACATCTTCTATGTGAGTTAATTACAATGAATATCCCAAT | 1264 |
| core_89 | TTGAGATGGTTTAATTTCAACTTTTTGCACCAGTACCTACTCGTAT | 1265 |
| core_90 | TGGCTGACCTTCACGTATATTTTTACAGTTAATGCCCCCT | 1266 |
| core_91 | CCTCAGCAGGAATATTTTTGTGTATCACCGTACTCAGGAGTCAGAACC | 1267 |
| core_92 | GACCTAAATTTAACGGAGTGAGAATAGAACGTCCATTAATGGAAACA | 1268 |
| core_93 | AAGAGGAAAGTATTAAGATTTTTGCTGAGACTCCTCAAGAAGAGGGTT | 1269 |
| core_94 | ATCAAGAAAACAAAATGAATAACCAGATTTTCTTAAAAATACCGAACG | 1270 |
| core_95 | AAGTTTATTGAGTTAAGCCCTAGCTATCTTACCGAAGCCCTTTT | 1271 |
| core_96 | GGAGAATTGGAAGTTTCGAAGGCAAACGATCTGCTAAACAACTTTC | 1272 |
| core_97 | AGAACGGTTTATCAAAATCATAGGTCCAGATTCAAGTCTTTAATGCGC | 1273 |
| core_98 | AACTGAACAGAGAGAATAACATAAACCGGATATAAAACGA | 1274 |
| core_99 | GAATTACCTTTTACCTTCATTACCCAAATTGTTTAACGTCAAAACAG | 1275 |
| core_100 | AAGTACAATCGAAATCCGCTCGGAACCTATTTTTTTTTCTGAAA | 1276 |
| core_101 | TAACGTATGAAAATAGCAGTACATAAGAGGACTAGGAATCCAATCGCAA | 1277 |
| core_102 | GAACTGAGTGAGGCTTTACAACAACATACCGATAGTTGCGCTGAGAGAC | 1278 |
| core_103 | GAATACAATACCAAGCGCTTTTTAAACATCTGTATTTTTTGGGATTT | 1279 |
| core_104 | TAATTTTAAAAGTTTGATTTTTTAACATTATCATTTTGCGGAAC | 1280 |
| core_105 | CCAAGGTGAATTACCTTATGCGATTTTTTTTAAGAACTGGCTCATTA | 128 |
| core_106 | TTTTTGAAGAAGTATTAAACCAAGTTTTTACCGCACTCATCGAG | 1282 |
| core_107 | TACCTTTTTAACTTTTTTCCGGCTTCAGAATCAATAATCGGCTG | 1283 |
| core_108 | ATCAAACTTAAATTTCGTTTACCAGCTGGAGGTGTAAGGAGCGGGCGC | 1284 |
| core_109 | CAGTATAAATATGCGTCCGACTTGCGGGCCCCGCTTAATGGGAGCTA | 1285 |
| core_110 | AGCCATATTATAATTATTCATTTTTTTCAATTACCTGAGC | 1286 |
| core_111 | ATAAAGTACCGACAAAAGGTCGCCATATTTTATCAA | 1287 |
| core_112 | AAGCGACCACAATCCGCCGGGCGCGTCTTTGCTTTGTAGAAGCAAGGAT | 1288 |
| core_113 | CCGGGTACGCCAGCGGCAGTGTCAAACATTATAGTAGTAGAGATACAT | 1289 |
| core_114 | AGCCAACGAGCAAATAATCATTAGGCCGATGAGCTGAA | 1290 |
| core_115 | CTCAACAGTAGGGGTTGAGGATCCTATCCGCTTAAAGGTTGTTGCGGT | 1291 |
| core_116 | TTGCTTCTGTATTTTTATCGTCGCTAAAACAAA | 1292 |
| core_117 | TGAGAGTCTGGAGCAATAATGCCGGTCAAATCAACCTGTTAAGGTGG | 1293 |
| core_118 | ATTACTAATTCCACACACGATTTAGGAAAGGAAGGGAAGA | 1294 |
| core_119 | GGAGCGGAATTATCATTCAATATCTGCTTTCCAGGAACGGAGCCGTTT | 1295 |
| core_120 | CCCCCTGCGTAATGGGCACAGAAAAAGCCTGTTTAGTATC | 1296 |
| core_121 | TTAATTAATTTTTTTTTCCTTAGAATCCTTGAAAACAT | 1297 |
| core_122 | TTTCACGGTGCGGCCAGAATGCGGCTACTAAT | 1298 |
| core_123 | CAATAAACCGGGCCGTGCCTCCTCATCCAGCGTGCCGGTGATTTCAAC | 1299 |
| core_124 | AAAGTAATCAGCTAATGCAGAACGCGCTAAGAGAAT | 1300 |
| core_125 | GAATCTTAGCACCCAGCTACAATTCATCATCAGTTGAGA | 1301 |
| core_126 | TAAATAAGGATATTTTAGTTTTTTAATTTCGCGAGATTTTTAACTTT | 1302 |
| core_127 | AGCTTGACGGGGAAAGTTTATCCTAACACCGGTTGAGGTTTTGAAGC | 1303 |
| core_128 | TTCTAAGAAACAAGCATACGCCAGCCACCGAGTTGTAGCA | 1304 |
| core_129 | ATCCGGTATTCAAATGTAATTTAGGAGGTTGTTTTTGTTATATAAC | 1305 |
| core_130 | AAATAATACAATAGATAAGTCCTGAACATGTTTCTGTCCAGACGACGA | 1306 |
| core_131 | TCCCATCCTAATTTATGGGGCGCTAAAGGGATATAAGGCC | 1307 |
| core_132 | TAGAAACCAGGCATTTTCGAGCCAGTAACTGTTAACAACATCGTAGG | 1308 |
| core_133 | TTTACATCGGGAGAATTTTTCAATAATATTAGACTTTTTTTACAAACA | 1309 |
| core_134 | TTCGCAAATGGTCAATACCATCAAAATACTTTTGCGAGGACCGCGCC | 1310 |
| core_135 | TTTAGAAGCGGATTCGCCTTAAAACGAACTAACATCAATA | 1311 |
| core_136 | TTGACGGGACACCAGAATAATCCTGATTGTTTGTACCAGTCTGCTTTGAAT | 1312 |
| core_137 | CGTCAGCGCGGCCAGAGCAGTTGGGCGGTTGGCATAGTA | 1313 |
| core_138 | AAATCTACGTTAAGATAGGACGTTGGGAAGAATCAACTAA | 1314 |
| core_139 | CATAACGCCAAAAGGAATTACGAGTGTACATCGGCAAACG | 1315 |
| core_140 | AGAGACGCAGAAACAGCGGAGGGCGATCGGTGCGGTATCGGCC | 1316 |
| core_141 | TACCACATAAATAGTTGCTATTTTCCAACGCTAACGTTTTTGCGTCTTT | 1317 |
| core_142 | CCGCCTTTTTTCAACAGTGCCAGGTTATCTATTTTTAATATCTTTA | 1318 |
| core_143 | TCAGCAGCTGCCGCCAGCACATCCTCATAACTTTTAAATAAAAATTT | 1319 |
| core_144 | TGGCAAGTGTAGCGGCCGGCGAAGGTCACTGAATCATA | 1320 |
| core_145 | TGGCAATTCGGAACAACGTACTATCGTTTTAG | 1321 |
| core_146 | CGTCAATAGATGACCTGAAAGCGTTTTTAAGAATACTGGCCAACA | 1322 |
| core_147 | CATTATTACTTAAATCAAGATAGAAGATGATGACGCGAGGGGTTGCT | 1323 |
| core_148 | TGGTCTGGTAGGGCGCAGAGCAACACTATCATAACCCTCTGCTCATT | 1324 |
| core_149 | GATTATACTTCTGAATAATTTTTTGAAGGGTTAGAACC | 1325 |
| core_150 | GCAGCCTCTGGCCACACCCGCCGTGCGGCTGCAATAGCA | 1326 |
| core_151 | TCGTTAGAGAAGGCTTCGAACCTCTATACAACGTTAAATAAGAATA | 1327 |
| core_152 | TATAACGTGGTCAGTAGCATCACCTTGCTGAATTAACCG | 1328 |
| core_153 | CAGATATAATCAGAGCGCGCCGCTTGACGTTTTTTCGTCTCCGCACAG | 1329 |
| core_154 | CAGGTAGAAAGATCGCTGCGCGTAACCATGCATGAGCCGCGTGGCGA | 1330 |
| core_155 | TCAGGAAGAATCAGAAAAGCCCCATTCCCAATAAAGAATT | 1331 |
| core_156 | GCAATGCTCACATGTCAATCATGACCATTCATTAACA | 1332 |
| core_157 | ATACTTCTTTGATTAGCAAATGAAGAAGTGTTAATACATTTGAAGCACG | 1333 |
| core_158 | GAAAGGAATTGAGGAACGCTGAGAGCCAGCAGTAATAACA | 1334 |
| core_159 | TAGCTATATTTTCATTTCACGCAAACCTCAAATTTTAGAC | 1335 |
| core_160 | AACCGCAATGCCGGACCGTCATAATACCTGCACGAGCT | 1336 |
| core_161 | TAAAAGAGTCTGTCCACGAGCATGTTATTTTCGCCAACATATTCTTAC | 1337 |
| core_162 | GAGATAGATCACTTGCCTGAGTAGAAGAATTTTTTCAAACTA | 1338 |
| core_163 | ACCCTTCTTTTATAATTCTTTCCTTTTTTATCATTCCA | 1339 |
| core_164 | CAGTGAGGAATCCTGAAAATCTAATGGCAAATCAACAGTTAAAGAAAC | 1340 |
| core_165 | CGGTTGATATCGCACTCTCATATAGGAGCAACTGTTGGGA | 1341 |
| core_166 | CATCAATTAACAAGAAATCAGACGACACTTAATTGAGAAT | 1342 |
| core_167 | GGCAAGGCTCTGCGAACGAGTAGATTTAGTTT | 1343 |
| core_168 | ATGTACCCAATCGGTTTTAGAACCCCAGCCAGCCATTCAGGCTGCACG | 1344 |
| side1_protrusion_1 | GTTTACCATGCCAAGCTTTCAGAGGTGAACAATCGGTCACGACAATTAGAG | 1345 |
| side1_protrusion_2 | CAATAGGAACGCCATCCAGCTCATATTCGCATGTAGCTCA | 1346 |
| side1_protrusion_3 | CACCACCCTACATACAACAAAAGGCATTACCAACTTGAGCTCAACATT | 1347 |
| side1_protrusion_4 | CGAACCAGCCATGACCGTAATGGGTTCGCGTCTACAGCGCCAT | 1348 |
| side1_protrusion_5 | CTGGTGCCGGAAGACTTCAAATATTAGATGGGTTGTTAAATTTTTAAC | 1349 |
| side1_protrusion_6 | AAATGTGAGCGAGTAAAGCTTTCACATTTGGGGCGCAGTC | 1350 |
| side1_protrusion_7 | GGAAGGTATACAGGAGTCTGAATTAAGCCAGACGGGAAC | 1351 |
| side1_protrusion_8 | GGATAACCTCACCGGAGAGCCGCCAATGGAAACAGACGGTTGTTACT | 1352 |
| side2_recess_1 | TCGCGCAGAGGCGTTATACAGTAAGTAAAACAGAAAT | 1353 |
| side2_recess_2 | GGGACATTCGTGGCACAGGTGAGGAACAACTGCAGAAGA | 1354 |
| side2_recess_3 | CTGTAGCGATTAGACGCAGAGAGACAGCATCGATTCGGTC | 1355 |
| side2_recess_4 | AAAGAAATTGCGTTAAATCCTTTGCCCGATTCGACA | 1356 |
| side2_recess_5 | TAAAACAGAGACAATAAACCACCAAATAGATTAGAGC | 1357 |
| side2_recess_6 | AATCACCGGAACCAGAGCCACGCCGTTTTCATGCCTTTAGCGTCAGA | 1358 |
| side2_recess_7 | CCGCCGCCTGACAAGAAAACAGGGTGCCACTACCATTAAACGGGTAAA | 1359 |
| side2_recess_8 | CAGTGCCTCAAGAGTAATCTAGCATTGAAGAACCACCACCAGAG | 1360 |
| side3_protrusion_1 | AAAACTAGACCGTTCTAGCTGATAAATACAAGAGATGTGTAGG | 1361 |
| side3_protrusion_2 | CAAAAGGGTGAGACAATCATTGCCGCGGCCTTTAGTGATGAAGGG | 1362 |
| side3_protrusion_3 | GGAGACAGAGAGGGTAGCTATTTTTGAGAGATCTAC | 1363 |
| side3_protrusion_4 | CGGTCTTATGCTGATTGCCGTTCCGACATAAAAAAAT | 1364 |
| side3_protrusion_5 | CCCGTAAAAAAAGCGTCGCTGTAAAGATTAACGGAGAAGCCTTT | 1365 |
| side3_protrusion_6 | GACCCTGTTATGATATCTGAGTAAATCGATGAACGGTAATCGT | 1366 |
| side3_protrusion_7 | TAAAGTTAAACGATCAGATGATGCAGATATTTAGGAA | 1367 |
| side3_protrusion_8 | AAAGGCTATCAGGACCCTCAACATATTCCACAGGGCG | 1368 |

TABLE 10
T = 4 (equi)

| | | SEQ ID |
|---|---|---|
| Name | Sequence | NO: |
| core_1 | GTTAGCAAGAATACCCAAAATTTTTAACTGGCATGATTGAAACAATGAA | 1369 |
| core_2 | CTGTAGCATTCCACAGATTTTTAGCCCTCATAGTTAGCGTAGCTATTAA | 1370 |
| core_3 | AACTAAAGGAATTGCGTAGTAAATAAGTTTTGTCGTCTTTAAGGAGCC | 1371 |
| core_4 | TTCGGATTATACATTTATTCTGTCCAGACGGGCGCTAGGGAAAACGCT | 1372 |
| core_5 | TACAAGTTTTAACGGTAAAGTAAACAATTTCATTTGAATCGTTGAAA | 1373 |
| core_6 | AGGTGAATTGAGCAAAAGAAGATCAAAATCGCATCACGCTTTGCCACGC | 1374 |
| core_7 | CGAGCATGAACAACATGTTCAGCTACAAAAGGGGTCAGTGTGCCCCCT | 1375 |
| core_8 | CGTCTGAACAACAGGACGCTGGCACTACAGGGCGCGTA | 1376 |
| core_9 | AAGACTCCGTAATAAGATCGCAAGTATGTAAAGCTTCTGTAAATCGTC | 1377 |
| core_10 | ACGCGCCTAAAATAATCATCGAGAACAAGCAAGCCGTTTTAACATGTA | 1378 |
| core_11 | AGAGCGTTTAACGTGCTTTCCTCGTCGCGCTTAAACATCAC | 1379 |
| core_12 | ACGATCTAGAATTTTCTGTATTTTTTGGATTTGAGAATA | 1380 |
| core_13 | TAAACATCCCTTACAAGTTGCGCTCGGAACGTAGAATC | 1381 |
| core_14 | GAAAATTCATATGGTTTTTTTTCCAGCGCCAAAGACAAAA | 1382 |
| core_15 | AAAGGAACCGCGAGAAAATTTTTTTTTTCAAATATATAGCCAAAATCA | 1383 |
| core_16 | TTAATTTTCCCTTTTTTTGAATCCTTGAAAACATAGCGTAGGTCTG | 1384 |
| core_17 | CTATGGAATACCGTTGTCATGGAAACAGAGGCGGTCAGTATTAATTTAGG | 1385 |
| core_18 | ACACCCGCAGGGTAGCAACGGCTAAGACAGCACGACAATGA | 1386 |
| core_19 | GAAACATGAAAGTAATGGCATTTTGGAACCAGAGCCACCA | 1387 |
| core_20 | AGTGTAGCGGTCACGCACGTGGCGTTTACATTACAATATT | 1388 |
| core_21 | TATCTTACCGCGTTTTTTTTTCATCAAGAGCAACCGTATAACAAATCCA | 1389 |
| core_22 | TAGCAATAGCTAACCCACATTTTTGAATTGAGTTAAGCCCCCAGACGT | 1390 |
| core_23 | AGGCTGAGGTTTTGCTCAGTACCAGTTTTTCGGATAAGTGCC | 1391 |
| core_24 | CAAGAGAAGATGAAACAAACATCCTATCGGTTTATCAGCTTGCTTTCG | 1392 |
| core_25 | CGCAGAGGTTTGAATACCAAGTTAGGATTAGGTATTAGCGAAAACCTAT | 1393 |
| core_26 | AGAGACTACACCGGAAATCTTTTTTTTGACCTAAATTTTGAATCTT | 1394 |
| core_27 | TCTTTAATCGCTCAATTAAAACAGAGGTGAGCCGCCGCCA | 1395 |
| core_28 | TATTCTATCTCAAGAAAACAAAATTTTGCTTTGACGAAGTCTGTC | 1396 |
| core_29 | GAACGTGGACTCCAACGTCAAAGCTCACAATGCCGGCGATGCGCGTA | 1397 |
| core_30 | AGTTTTTTCGGGGAAATCCACACATGGTCATAGCTGTTAACGGCA | 1398 |
| core_31 | TAGAAACCCCAAGAACGGGTATTAAACCAAGTACCGCACT | 1399 |
| core_32 | TGAGAGCCAGCAGCCTGATTGCCGAATTATTCAGGAGCTAAACAGG | 1400 |
| core_33 | AAATGAAAGGCCACCGTTAGTAATATGCGCCG | 1401 |
| core_34 | TAAGAATAAACCTTTTTTTTTTACCTCCGGCTTAGGTTGGGTTA | 1402 |
| core_35 | GTGAGTGATTTTTTCATACCTTTTACAGTTAACCTTGAGTCCACCCT | 1403 |
| core_36 | ACGTAGAAAACCGCCACCTTTTTTCAGAGCCAAACAGTGCGGGCGACA | 1404 |
| core_37 | GGGGGTTCGTAATCAACATACGAGCCGGAACTATTAAAGGTGCCGT | 1405 |
| core_38 | AATCACCCGGTCATAGCCCCCTATTAGCGGGACTCCT | 1406 |
| core_39 | GGAAGGTTATCTAAAATATCCACTTCTTTGAAGTAAAAGGCACGTAT | 1407 |
| core_40 | AGAATATAAAGTACCGAATGCAGATTCGATGATACAGGAG | 1408 |
| core_41 | GACCGTGTGATAAATACATATTTAACAACGCCTATTTTCA | 1409 |
| core_42 | TTTTATCCTAAGTCCTGGTAAATATTGATTTTTGGAAATTATTC | 1420 |
| core_43 | ACCAACGTTTTTTAACGAGCGTCTTTAGTTGCTA | 1421 |
| core_44 | AGCACTAAGCCATTGATGGATTAAGAAAGGACTACGTGA | 1422 |
| core_45 | CAGTAGGGCTTATTTTTTTGAGAATCGCAGGCGTTAA | 1423 |
| core_46 | ACAATAGAAATGGTTTTTATTACTTTTTGCAGTATAGCTTTAGTT | 1424 |
| core_47 | GAGGCAGGTCAGACGATTGTTTTTCCTTGCCATTAGCAAGGCCGG | 1425 |
| core_48 | AGGAGGTTCAGAGCCGCCACCAGAATAATCAAGCCTAT | 1426 |
| core_49 | AAGCGAAAGGAGCGACATTTTGAGCGTTACCGTTCCAGTAAGCGTC | 1427 |
| core_50 | CACCATTAATATTCATTTTTAAACAAATAAATCCCGATTGAG | 1428 |
| core_51 | CAGCTACAATCGTAGGAATCATTACCGCGCTTTTTCAATAGCAAGCAAA | 1429 |
| core_52 | ATCCCATCCTAATTTAATACATGGCTTTTAACTCATTAA | 1430 |
| core_53 | AGCCAGAATGGAAAGCGCATTGACTGTACTGGTAATCTACGACAATA | 1431 |
| core_54 | GCAGTCTCTGAATCGAACTGATAGCCCTAAAAGCAGAAGA | 1432 |
| core_55 | CATCGCCATTTGAATGGCTATTAGATGGCCCAAGGGAAGA | 1433 |
| core_56 | ACCGTCTATCAGGGGGGTCTTTCCTTATCATTAATCAATAATCGGCT | 1434 |
| core_57 | AGCAATACCGCCTGCAACAGTGCCATCTTTTCACCACCAC | 1435 |
| core_58 | AAGAAATTGCGTACGGCACCTTGCCAGTTGAAAGGAATTG | 1436 |
| core_59 | CAACAACCATCGCCCACTTTTTCATAACCGATATATTCGGTTGCCCTGC | 1437 |
| core_60 | ACCTGTTTAGCTATATCCCAGCAGGCGAAAATTCCACGCTGGTTTGC | 1438 |
| core_61 | ACCATTAGATACATTTCGCAAATGAATCGTCAGCCTCAGCAACGCAA | 1439 |
| core_62 | CGCTGAGGTCGTCACCCTCAGTTTTTAGCGAACAGAGGC | 1440 |
| core_63 | ACACTCATGAATGCCAACGGCAGCGTCAGCGGGAGCAAAAATGTGCCGG | 1441 |
| core_64 | CGACAACTGATAATACATTTGAGGCAGTTGGCTTATTT | 1442 |
| core_65 | GCATAAAGTGTATTTTTAGCCTGGGGTGTGAGGATCC | 1443 |
| core_66 | CGTATTAAATCCTTTGCCCTTTTTAACGTACCGAACGAACCACCA | 1444 |
| core_67 | CAGTGAGACTGTTCTTGCTGGTATTTTTATATCCAACCTCATACC | 1445 |
| core_68 | TGGTTTTTGCCCTTCATCCCTTATAAATCAAAAGAATAGCACTCACAT | 1446 |
| core_69 | TTTCCCAATTGTAAATAACCAATGCTAAATCGGGGCGCG | 1447 |
| core_70 | GCGATTAAGTTGGGTAACGCTCCTCGTAAAACATTGCCTAGATTCAA | 1448 |
| core_71 | AAATCAATGAACCTCAAATATCCAGTACCTAACGTCA | 1449 |
| core_72 | CGGGTACCGAAGCGGTGTTTTTCGGTGCCCCCTGCATCAGACGA | 1450 |
| core_73 | TGAGCCTCCTCACAGTCCTAATGAGTGAGCTACCGAGATA | 1451 |
| core_74 | GCCATTCGTATCAGGTCTAGCATGCATATATT | 1452 |
| core_75 | GAACAATATTGCCTGAGTAGTTTTTAGAACTCAAACTAAATCCTGAGAA | 1453 |
| core_76 | GGAGAAGCCTTTATTTAGCATAAAAGGAACGCGGCGGATT | 1454 |
| core_77 | GGGAAACCTGTCGTGCCGCCAGGGGGCAGATGATGGCAAT | 1455 |
| core_78 | AAACCAGGCAAAGCACGGAACGGTCTGGTCAGCTCACCATCAATAT | 1456 |
| core_79 | TGGTGCTGTGCCGGACTTGTAGAATACAGTAAAAACCCTCCAAAAAACA | 1457 |
| core_80 | CTGCAGCCGCTCGAATTTCTGTTTTTCAGCACGCGTGCCCTAAAGG | 1458 |
| core_81 | GATGAATACACCGTCGGTGGTACGAAAGAGGCAAAAGAATACACTAAA | 1459 |
| core_82 | CAAAAACATTATGCGTTTTTGTTGGTAGGAGCGGAATTATCATCAT | 1460 |
| core_83 | TCGGCCTTTTCCAGTCCTGTGGTGAGTGTCACTGAGCCGGGTCACTGT | 1461 |
| core_84 | CAGGTTTTTTACATCGGGAGAAACATTTTTTAACGGATTCGC | 1462 |
| core_85 | GAAATAAATGCCATCCCACGCAACCATGTGTAGGTAAGAGAGTCT | 1463 |
| core_86 | TGTTTTTATAAGGCCGATTTTTTTAAGGGATTTTAGACAGGGCCGCTT | 1464 |
| core_87 | AAGCACTAAGGGTTGAGTGTTGTTCCAGTTTTTTTGGAACAAGAGTCCA | 1465 |
| core_88 | ATCAGTGAAATCTATTTTTAAGCATTACGCCAGGGGTTAGATGTGCACT | 1466 |
| core_89 | GGCTGGTAATGGGTTTTTAAAGGTTTCTTTGCTCGTCACGGGTTAC | 1467 |
| core_90 | GGGCGCGGTCCATTAAAGCGGGGTACCTACCACTTCTGAAGTCAATA | 1468 |
| core_91 | AACGGTAAGGCACCGCTTCTGCAATATCTGGTATTTAGAA | 1469 |
| core_92 | TTTAAGAACTGGCTCAAGCGCGAAACTGACCGGAGACA | 1470 |
| core_93 | CCGTGCATGACCGTAATGGGATAGTTTCATTTGGTTGTAC | 1471 |
| core_94 | GTCAAAAGCAAGGGTGAGAAAGGCCAGAACCCTTCAATCAT | 1472 |
| core_95 | AAATTTTTAACTTTGAAAGAGGACGGGAACCGAACAAAGTA | 1473 |
| core_96 | CCGCCTGGCCCTGAGAATTCCTGATTATCCAAAACATTA | 1474 |
| core_97 | GCACGTGAGTTACGGCTTCGGCCAACGCGACCCTGTAATATTTTGTTA | 1475 |
| core_98 | ATTTCCTGATTGTTTTTAATGAAGGAGGTGTCCAGCATCACGGGTAA | 1476 |
| core_99 | GAGCCCCTTTTTGATTTAGAGCTTGAGGGGTCGA | 1477 |
| core_100 | GGCAGATTCACCAGTCATTTTTACGACCAGTAATAAAAGG | 1488 |
| core_101 | GAGTTGCACGGTTTGCGTATTGGGCAGCTGCAGGATTATATATCAAAA | 1479 |
| core_102 | GCAAGCGGCCTGTTTGATGGTGGTTCCGAAATCGGCAAAA | 1480 |
| core_103 | TGAGTAAGCAATCGATGAAATTCGCGTAGCACTCCAGCCAGCTTCAGGGT | 1481 |
| core_104 | GCAACAGGGGTAATTTCATTGAATCCCCCTTTAGTTTGTAGTAGCA | 1482 |
| core_105 | ATCGGAACCGGGCAACCTGACCTGAAAGTTTTTGTAAGAATACG | 1483 |
| core_106 | TTAAAAATTATTAATTTTTTTTAAAAGTTTGAGTCAGAGATA | 1484 |
| core_107 | TTACCGCCACAACTAATATTTTTATTAGAGCCTAATGGAAGACATTCT | 1485 |
| core_108 | TCATTTTTCGTTAATACTTTTGCGTTAAATGCAATGCC | 1486 |
| core_109 | TTGAGGACAGAATGCGGCTTTTTGGCCGTTTTCACGGACCTCAGATGC | 1487 |
| core_110 | TCATTTTGCGGAACAAAAACAATTTCATCAATATAAAAAGGGAGAGG | 1488 |
| core_111 | GTCACGTTAAATCAGCCCTCAGGAAGATCTTAGACTTTAC | 1489 |
| core_112 | AGAAACCACCAGAGTAGATGGGCGCATCGTAAAGTATCGG | 1490 |
| core_113 | TCAATTCTAGCAATAAATAAATAAGTAAAATGTTTAGAGAAGAAA | 1491 |
| core_114 | TAAAGACTTTTTCATGTTGCGGGACTTGCAGGGAGTTAAAGCACCAAC | 1492 |
| core_115 | AAAGAAGTTTACGGAACTTTTTACATTATTACAGGTAGAAAGAT | 1493 |
| core_116 | CGGTCCGTAACCGCCAGAACCGGATATGGAGAATTAACTAAAAGTAA | 1494 |
| core_117 | CGTTCCGTGGCAGCCTCCGGCCAGATTTTTCACATCCTCATA | 1495 |
| core_118 | TAGACGTCAAAAGTTACTATAAAAGGCCCGATAGCAGCACCGTACCGCCA | 1496 |
| core_119 | GGTAAACTTGACAACCCTCAGACAGGGATAGCAAGCCCAATAGGAA | 1497 |
| core_120 | TAGGTGTAGGTTGATATAAGTATATTTTTCGTGGTGCGGGCCGATGAAG | 1498 |
| core_121 | AAGGGATAGCTCTAATTAGAGCCAGCAAAATCAAACGTCA | 1499 |
| core_122 | TCAACTTTAATCATTTTTTGTGAATTACCTTATGCGATTTAATAAA | 1500 |
| core_123 | TGCCTTTAGCGTCAGTCGAGAGTCACCGTACTCAATATCAGAGAGA | 1501 |
| core_124 | GGTGTACAAACGCCAAAATTTTTGAATTACGAGGCATTAAAATCTACG | 1502 |
| core_125 | ACCAGTAGCACCGTCACCGACTTGTCCCGACTAGCCATAT | 1503 |
| core_126 | ATAGCTTAGATTAAGAGTACAAAAACTTTCAGTAATTG | 1504 |
| core_127 | GGCGTTTTAGCGAACCTTAATTGCTGAATATACGGATGGCTTAGAGC | 1505 |
| core_128 | CTGCCAGTTTTACCATTTTTTCCCGGAATTTGTGTTAAATGT | 1506 |
| core_129 | ACCAGAACCTTCATCATGCTCATTAAAAAAGCACATCGACCGGCCAG | 1507 |
| core_130 | CTTTACAGACAGTTTCAGCGGAGTTGCTAAACCTACAACGC | 1508 |
| core_131 | AGAAGGCTTATCCGGTATTCTAAAAATTCTTTAATTTGCATGAAAAT | 1509 |
| core_132 | GACCAGGCGCATAGGCGGAACGAGCGACCTGCTCCATGTTCCACCACC | 1510 |
| core_133 | GACTTTCTCCGTGGTGTAACCTCACTCATTTGCCGCCGCAAATATCG | 1511 |
| core_134 | CAAATGCTTTAATTTTTCAGTTCAGAAAGGCTTTTGC | 1512 |
| core_135 | CCCATGTATACCGCCACCCTCAGGCCCGGAAGCAGATAGCCTTCAAGTT | 1513 |
| core_136 | TTGTTTAACGTCAAAACAGTTACAAATCAATATGAATTAT | 1514 |
| core_137 | CAACGGAGATTTGTATCTTTTTTCGCCTGATAAATTGTGTCGTTTAATT | 1515 |
| core_138 | GAAATCCGGCGCAGACGGTCATTTTTTCATAAAGATGAA | 1516 |
| core_139 | TATTTATCCCAATTCAAAGACACGGGCACGGAAAAAGAGACGCAGA | 1517 |
| core_140 | AAACAGCAGTTGGGCAAGAGGAAATCAACGTAACAAAGCAGAGTAAT | 1518 |
| core_141 | TGAGAGATCTGATATTCAATTTTTCGTTCTAGCTGATAAAACTTAGCC | 1519 |
| core_142 | CCCTCAGAATACATATTTGTCACAAATAAACTGCGGGAG | 1520 |
| core_143 | CGGAACCGCCTCCCTCAGAGATCAACCGAGGTTTTTAAGGAACACCC | 1521 |
| core_144 | AGGCAAATTTTTAATTAGCAAAATTAACTAATAG | 1522 |
| core_145 | AACTCCAAACCAGACGAACAGGATTTTTAGATTGTGGAAGTAAGA | 1523 |
| core_146 | AACCAGACAGTACCTTCTAAAGTACGGTGTCTGGAAGTTTATCAAAAA | 1524 |
| core_147 | AATCGGCGAAACGTACAGCTTTTTCCATGTTGAGGGGACGACGAC | 1525 |
| core_148 | CCGGAAACTGCCAAGCTTTCAGAGCGCCAGCTCGGCCT | 1526 |
| core_149 | GGCGAAATTGGGAAGGGCGATCCTCGTCGCGCAAACG | 1527 |
| core_150 | GACTGTAGCGAAGCCCAAACGCAAACATAAAA | 1528 |
| core_151 | ACGAACTATGCCAGAGCTATCTTTTTTAACCCTCGTTTTACAGGCA | 1529 |
| core_152 | AGCGCTAGGTGAACAAAGTCAGAGGAGAGAATATAATAACG | 1530 |
| core_153 | AATTTCTCATAATTTAAAGCCAACGCTCAATCAGATATTTTTGCAC | 1531 |
| core_154 | AACCAAAATAGCGAGAACGAGAATGACCATAACATTCCAT | 1532 |
| core_155 | CATTTTTGATGCTGTAGCTCAACATGTTTTAAATATGCAA | 1533 |
| core_156 | GTTAAACGATGCTATGCGCAACTGGGGGGATGTGCTGCAA | 1534 |
| core_157 | CATCAAAAATAATTCGCTTTTTTCTGGCCTTCCTGTAGCC | 1535 |
| core_158 | ACAAAGGCCCATTCTTTTTAGGCTGCCGGAGAGATCCCGTACACATTCA | 1536 |
| core_159 | CAGAAGGAAGTAGCGACAGAACTTCGCTATTAGTGGAGCC | 1537 |
| core_160 | TAACATCCAATAACAGTTGATTCCCAATTCTTTTTGCGAACGAGTAGAT | 1538 |
| core_161 | ATAAATCACAGGTCAGAACAACCCGTCGTTTTTATTCTCCGTGG | 1539 |
| core_162 | TAATTGCTCCTTTTGAAACAGCGGATCAAACAAGAGATA | 1540 |
| core_163 | TAAGAGGTCGTTTTAATTCGAGCTAAAAGATTGGTTGTGTCGCACAGG | 1541 |
| core_164 | GCCCCAAATCAGAAGCACTAATGCTGAGATTTAATTGGGCTTGAGATG | 1542 |
| core_165 | TCAAGATTCCAGAGCCACCAGTAACTAGAAAAAGCCTGAATTTAT | 1543 |
| core_166 | ATAAGCAAATGTACCCCGGTTTTTTGATAATCAGAAAAGGTAGCTATTT | 1544 |
| core_167 | TTAGTGAACTTACCCAAGCCCGAAAGACTCCAAATAAGAATGGCAACA | 1545 |
| core_168 | ATATTTAAGTCACGACGTTTTTTGTAAAACGAATAAAAAAAGCTTTCA | 1546 |
| core_169 | AGCCATTTCACGGAATCCAATGAAACCATACGGGAACGGA | 1547 |
| core_170 | AAGTTTATCATAAAGGACGATTTTACAGGGAAGCGCAT | 1548 |
| core_171 | AAAGCGGATTGCATCATCAAAGCGTCAACTTAAATTTCTG | 1549 |
| side1_recess_1 | ATTGTTATCCGGGCGAAAAACCATCACCCAAATCATGGCACAG | 1550 |
| side1_recess_2 | AGCAAATCGTTTCCTGTGTGAA | 1551 |
| side1_recess_3 | CAATTACCTTCTTAAACAGCTTGATACCGATCTGGTGTGTTC | 1552 |
| side1_recess_4 | GGAGGGAAGAACAAGAGTTTATCAATTTAGGCAGAGG | 1553 |
| side1_recess_5 | CATTTTCGAGCCAGAAATACCTATAACTAACAAAGAA | 1554 |
| side1_recess_6 | AATAATAAATAACCTTTGCTGATGTTAATGGAAACAGTACATA | 1555 |
| side1_recess_7 | AATCAATATATTTTAATTGAAAAAAAAGGCTCCAAAATATTAAG | 1556 |
| side2_recess_1 | CCAATACTGCGGGTCAATAAGCTGAAAAGGTGGCAGAACAAAC | 1557 |
| side2_recess_2 | CAGGACGTTGGCTGGATAGCGT | 1558 |
| side2_recess_3 | AACCGCAACTTTGACCCCCAGCGATTATACCATTATACCAGT | 1559 |
| side2_recess_4 | GAACCCTTAGCTGATTCTTTTCACTAATTGCGTTGCG | 1560 |
| side2_recess_5 | CTCACTGCCCGCTCGCGTCCGTCCAGCGCCTGCGGCC | 1561 |
| side2_recess_6 | AGGAAGTTTTGCGGTATGCGCGCCCATTGCAGGCGCTTTCGCA | 1562 |
| side2_recess_7 | CTCAATCCGCCCTAAAACGTAATGCCACTACGAAGGAAGATTTT | 1563 |
| side3_recess_1 | ATGCGTTATACGAACGCGAGTTTTGAAGCCTTAAAATTAAAGG | 1564 |
| side3_recess_2 | AGTCAATAGTGTTTAGTATCAT | 1565 |
| side3_recess_3 | AGGTTTAGCCGTAACACTGAGTTTCGTCACCACGCTGAGAAG | 1566 |
| side3_recess_4 | GAGCGAGTGATTAGAGCGGAAGCATCAGGTCTTTACC | 1567 |
| side3_recess_5 | CTGACTATTATAGACGATAAATCATCAGTAGATACAT | 1568 |
| side3_recess_6 | TGGCTGACGAGTAGTAAGGAATACCAGTGAATAAGGCTTGCCC | 1569 |
| side3_recess_7 | TGACGAGAAACCTCATTTTACCGCCACCCTCAGAGTTAGATTGC | 1570 |

### Example 2—Design and Testing of Programmable Cylindrical Shell System for Virus Trapping

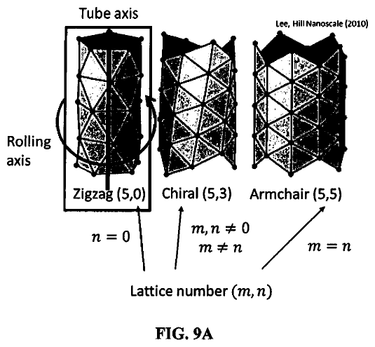

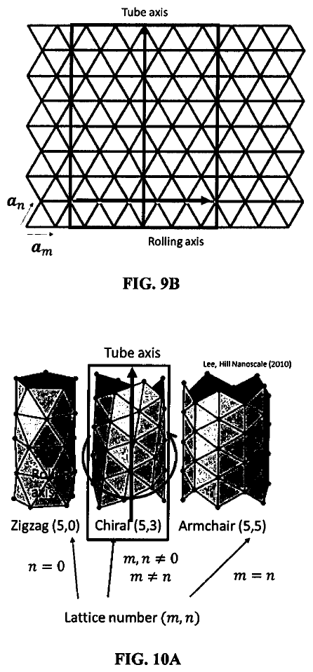

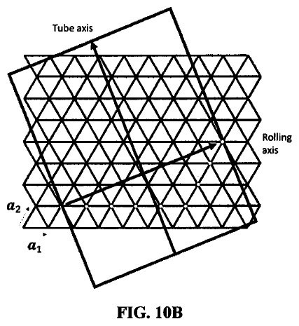

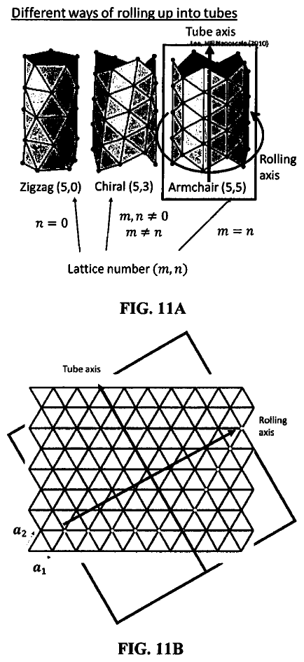

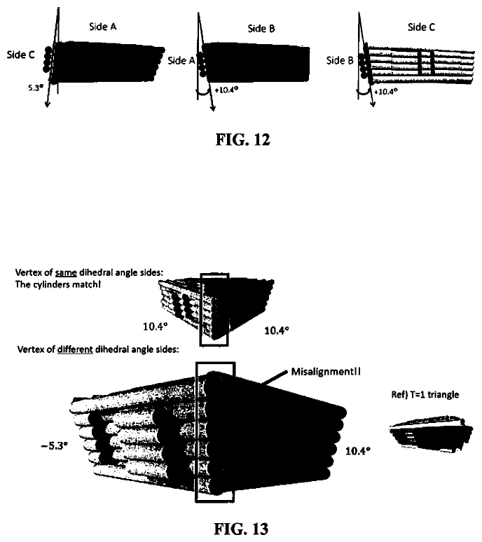

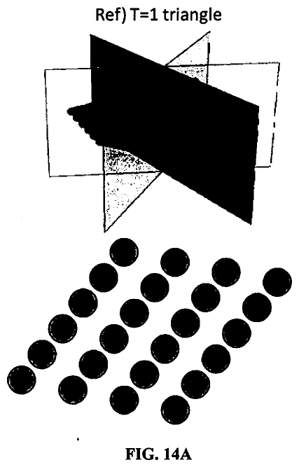

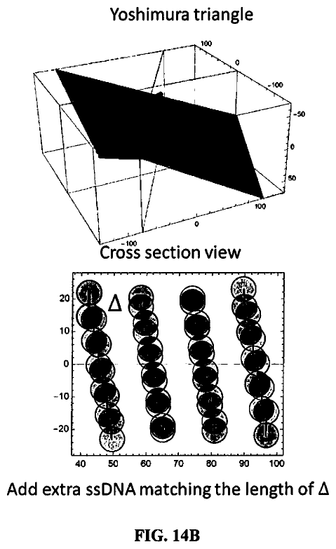

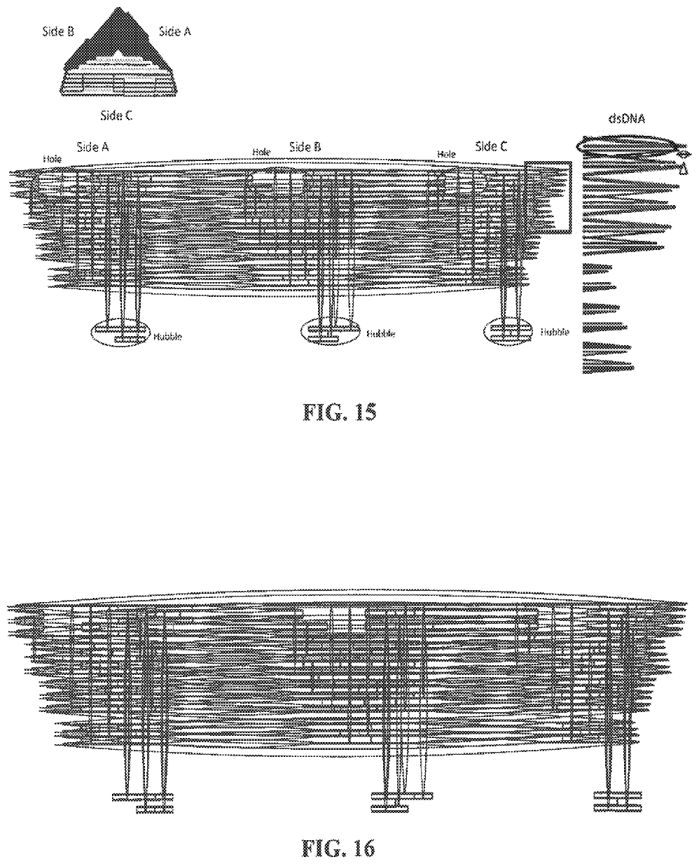

Using the approach illustrated in Example 1, including the scaffold strands identified therein, and the conceptual designs illustrated in FIGS. 9-16, and the nucleic acid sequence illustrated in Tables 11-28 below, triangular subunits that can self-assemble to form cylindrical shell systems will be prepared and tested against filamentous virus particles such as filamentous Influenza A virus particles and non-infectious Ebola virus-like particles.

TABLE 11
Core 1 Side 1; Side 1 Body

| | | | | SEQ ID |
|---|---|---|---|---|
| | Start | End | Sequence | NO: |
| Core 1 Side1 1 | 21[104] | 9[103] | TATAATCAACTATGGGTAAAGGTATGTCAA | 1571 |
| Core 1 Side1 2 | 17[96] | 6[96] | AGCCTTTATTTCAACAAAAGGGTGAGAATC | 1572 |
| Core 1 Side1 3 | 22[95] | 20[96] | CTGATAGCCCTGAGAAGTGTTTTTCCTTTG | 1573 |
| Core 1 Side1 4 | 10[167] | 1[183] | CCGATTGGCGTTTTCATCGATTTCTGCTCA | 1574 |
| Core 1 Side1 5 | 4[135] | 6[120] | AGTCACGACGTTGTAACCAGGCAGTGTAGGT | 1575 |
| Core 1 Side1 6 | 3[152] | 7[167] | ACCAGTCCCAGAGCCAGACGATTGGCCTTGA | 1576 |
| Core 1 Side17 | 15[96] | 9[111] | ACACTGGTGTGTTTCCACCATCATCACCGAC | 1577 |
| Core 1 Side1 8 | 11[72] | 0[56] | AGGAAGATATATTTTGTTAAAATTCGCATTAA | 1578 |
| Core 1 Side1 9 | 12[151] | 10[128] | AGCATCAGCGGGGTCATTGCAGGGTGCCGGGAAATTA | 1579 |
| Core 1 Side1 10 | 1[128] | 31[151] | GAACCAGAATCACCTAATCAGTAGCGACAGAATCAAGT | 1580 |
| Core 1 Side1 11 | 11[96] | 14[88] | CTCCGGCTCATATGTACCCCGGAAACTAGCTTCTTTGC | 1581 |
| Core 1 Side1 12 | 8[103] | 17[95] | ATATGATATTCAACCGACGACAGTAACGGCAGCGGGAGA | 1582 |
| Core 1 Side1 13 | 0[175] | 10[152] | TGATGAAGGGTAAAGTTAAACGACTTATTAGAGGGAGGG | 1583 |
| Core 1 Side1 14 | 14[127] | 19[135] | TTAGACGGAATTTGCCACTCAAACTTACCGCCAGCCATT | 1584 |
| Core 1 Side1 15 | 13[104] | 0[96] | TGGTAGAATATCACCGCAGAGCACTCTCGTCGCTGGCAGC | 1585 |
| Core 1 Side1 16 | 5[96] | 7[119] | CCGGCACCGCTTCTGGTGCCGGAAAAACGACGTCACCGCC | 1586 |
| Core 1 Side1 17 | 0[95] | 12[88] | TAAATTGTAAACGTTATGTATAAGCAAATATTGCGGTATG | 1587 |
| Core 1 Side1 18 | 8[79] | 10[72] | TGATAAATATGAACGGTAATCGTATTGATAATCAGAAAAG | 1588 |
| Core 1 Side1 19 | 14[87] | 13[95] | TCGTCTTCGCGTCCGTTGCCTAATGAGGGTCACTGTTGCC | 1589 |
| Core 1 Side1 20 | 30[151] | 11[159] | ATCTTTTCCCGGAACCTGCTGATTAACGTCAGCGTGGTGC | 1590 |
| Core 1 Side1 21 | 22[155] | 14[128] | GTAAGAATACGTGGCACAGACAATATTAGAGGGTAAGCGCA | 1591 |
| Core 1 Side1 22 | 13[120] | 15[135] | ACAAAGTCTTTGAATGACCGAGTAAAAGAGTTAGAAGAAGTTACA | 1592 |
| Core 1 Side1 23 | 1[120] | 3[135] | AACGTCACTTCATTAATTTGGGAATTAGAGCCACCAGAGCGAAA | |
| | | | C | 1593 |
| Core 1 Side1 24 | 19[96] | 7[111] | TATTAAAAATATCCATCTTCAGCAAATCGTTAAGGCCGGAGACAG | 1594 |
| Core 1 Side1 25 | 14[119] | 13[103] | GAGAATTAGTGAGGCCGCTATTAGTCTTTAATGCGCGAACTGCG | |
| | | | GC | 1595 |
| Core 1 Side1 26 | 9[112] | 12[96] | TTGAGCCAAGGTGAATCACCCTGACACTCAATCCGCCGGGCGC | |
| | | | GGTT | 1596 |
| Core 1 Side1 27 | 16[143] | 9[159] | TTATCCTGAAATAAACAGAGCCGCCAGCCGCCACCCTCAGAACC | 1597 |
| | | | GCCA | |
| Core 1 Side1 28 | 7[80] | 19[95] | GAGGGGACGTTCTAGCTTCATAAACATCCCTTCCCGAACGACAA | 1598 |
| | | | CTCG | |
| Core 1 Side1 29 | 6[119] | 20[104] | AAAGATTCGCAAGGATAACGAGCGGAACAATATATCGGCCTTGC | 1599 |
| | | | TGGT | |
| Core 1 Side1 30 | 16[135] | 4[144] | AATCTTATGACAGGGAGTAATAAGCGCCATTCGCCATTCAGGCT | 1600 |
| | | | GGCCTCTT | |
| Core 1 Side1 31 | 14[143] | 0[120] | AAACAGGGAATTGAGCTATTGACGACTTGTAGGCCGTTCCGGCA | 1601 |
| | | | AACGCGGTCCG | |
| Core 1 Side1 32 | 7[120] | 16[144] | GCCAGCATCCAACGCTAAAAATTTTTAGAACCCTCATATATTTTC | 1602 |
| | | | CCCTACAATT | |

TABLE 12
Core 1 Side 2; Side 2 Body

| | | | | SEQ ID |
|---|---|---|---|---|
| | Start | End | Sequence | NO: |
| Core 1 Side2 1 | 12[367] | 10[352] | TGAGCAAAAGAAGATTACATTTGGCTCCA | 1603 |
| Core 1 Side2 2 | 22[327] | 14[312] | GGCCAACGACGCTGAGGTCTGAGAGACTA | 1604 |
| Core 1 Side2 3 | 6[391] | 5[407] | AATTAAACGTATAAACAGTTAATGCCCCCT | 1605 |
| Core 1 Side2 4 | 22[375] | 13[359] | GAGCCAGCAGCAAATGAAAAATCAATATAC | 1606 |
| Core 1 Side2 5 | 17[320] | 6[320] | AGACAAAGAACGCGACAGAACGCGCCTGAT | 1607 |
| Core 1 Side2 6 | 11[288] | 9[287] | GGTGAATTCATATGGTTTACCATTAGCAAA | 1608 |
| Core 1 Side2 7 | 0[287] | 11[287] | AAGTTACCAGAAGCGCGGCAGCACCGTCGGT | 1609 |
| Core 1 Side2 8 | 12[319] | 12[288] | GAAAACATAGCGAGCAAGAAACAATGACCAT | 1610 |
| Core 1 Side2 9 | 17[352] | 7[367] | ATATTTTAGTTAATTCGACGACAGAGAATAT | 1611 |
| Core 1 Side2 10 | 13[304] | 21[319] | AGATAGCTTAGATTAAAGAGATAGTCACCAGT | 1612 |
| Core 1 Side2 11 | 8[303] | 10[304] | ATTTACGAAATACATACATAAAGGACAATCAA | 1613 |
| Core 1 Side2 12 | 8[383] | 14[368] | GAATCGCCATATTTAAAGTAGGGCTAAAGAAA | 1614 |
| Core 1 Side2 13 | 15[336] | 21[351] | AATTCTTAAATTTATCAAATGCTGAACCTCAA | 1615 |
| Core 1 Side2 14 | 0[319] | 11[319] | CTTTTTAAGAAAAGTACTATCTTACCGAAGCC | 1616 |
| Core 1 Side2 15 | 7[336] | 17[351] | GATCATTTTCGAGCCATTAGTATCTTTCAAAT | 1617 |
| Core 1 Side2 16 | 6[303] | 8[304] | TATAGAAGGCCAGAATGGAAAGCGCCATCCTA | 1618 |
| Core 1 Side2 17 | 13[288] | 8[280] | TTAAGCCAATGAAACGTAGAAGCATGTAGAAACTAA | 1619 |
| Core 1 Side2 18 | 14[311] | 22[292] | CCTTTTTAGCAATACTAACCCTTCTGACCTGAAAGC | 1620 |
| Core 1 Side2 19 | 21[320] | 19[327] | CACACGATTTACATTGGCAGATACCTACATTTTGACG | 1621 |
| Core 1 Side2 20 | 14[359] | 16[352] | ATTTTCAGGTTTAACCCAGTATAAAGCCAAAGCCTGT | 1622 |
| Core 1 Side2 21 | 10[335] | 9[327] | CCACGGAATAAGTTTATTTTGTCTGGCAACATATAAAA | 1623 |
| Core 1 Side2 22 | 3[384] | 6[376] | GATAGCAAGGATTAGCGGGGTTACAGTGCCAGTAATTC | 1624 |
| Core 1 Side2 23 | 19[328] | 7[335] | CTCAATCGTATTATAACTATATGGATTTATCAACAATA | 1625 |
| Core 1 Side2 24 | 8[327] | 17[319] | AAGAAAAATAATATCCAGTCTCTTAAATGCTCAATCGCA | 1626 |
| Core 1 Side2 25 | 17[336] | 19[359] | GAAAACTTATATGCGTTCTATCAACAGTTGAAAGGAATT | 1627 |
| Core 1 Side2 26 | 12[351] | 22[328] | GATGAAACGTCAGATGTAAAGCATCACCTAGGGACATTCT | 1628 |
| Core 1 Side2 27 | 15[328] | 9[351] | GGTTAACATCCTGAACGAAACGCAAAGATTGTATCGGTTT | 1629 |
| Core 1 Side2 28 | 30[375] | 9[383] | TCGTCACCGTAGCAACAGTTTTGTCACGTTGAACTTTTTC | 1630 |
| Core 1 Side2 29 | 4[367] | 2[360] | TACCAGGCCACGCCACCACCCTCAGGCTACAGAGGCTTTT | 1631 |
| Core 1 Side2 30 | 13[296] | 0[288] | CAATAATATAGAAAATAGCAATAGAGCAGATAGCCGAACA | 1632 |
| Core 1 Side2 31 | 7[352] | 4[368] | GTAATAAATAAACATAACGGGGTCAGTGCCTTGAGTATTGCTC | 1633 |
| | | | AG | |
| Core 1 Side2 32 | 12[335] | 0[320] | TCCCTTAGAATCCTTGCTATTAATTAATTTGCTTCTGTAAATCGT | 1634 |
| | | | C | |
| Core 1 Side2 33 | 0[391] | 11[391] | TCTGTATTTTAATGGAAACAGTACATTTGAATTACCTTTGGGATT | 1635 |
| | | | T | |
| Core 1 Side2 34 | 16[311] | 15[327] | GATGCAATCAAGATTACGATTTTTTGTTTAACCTCCGGCTTAGG | 1636 |
| | | | TTG | |
| Core 1 Side2 35 | 14[367] | 0[352] | TTGCGTAGAGTAACAGAAAAAAAAAACAATTTCATAAATCAATA | 1637 |
| | | | TAT | |
| Core 1 Side2 36 | 8[367] | 12[368] | CAACGCCATGCTTGAGGACTAAAGAAATCTCCTACCTTTTCAAT | 1638 |
| | | | TACC | |
| Core 1 Side2 37 | 20[351] | 13[335] | GGTCAGTTGGCAAGAAATGGATTACCAGTAATAAAATCATAGA | 1639 |
| | | | AGAGT | |
| Core 1 Side2 38 | 0[351] | 12[336] | GTGAGTGAGCGCCGACAAAGGAGCCTTTAACACAATAGTGAAA | |
| | | | CATCA | 1640 |
| Core 1 Side2 39 | 5[296] | 17[311] | AAATCAGACGTCATACATGGCTTTTTACCGTTCCAGTAAGTTAA | |
| | | | AATC | 1641 |

TABLE 13
Core 2 Side 3; Side 3 Body

| | | | | SEQ ID |
|---|---|---|---|---|
| | Start | End | Sequence | NO: |
| Core 2 Side3 1 | 16[527] | 6[512] | GCAAAAACGAAAGAGGCGAGAGAAAGATT | 1642 |
| Core 2 Side3 2 | 7[480] | 5[495] | CGTTTACACATTCATTCCCAATTCTGCGA | 1643 |
| Core 2 Side3 3 | 12[503] | 11[487] | CGTACAATACCAAGTTACAAAATGACAGGT | 1644 |
| Core 2 Side3 4 | 1[576] | 3[575] | AACGTCAAATCATTGTGAATTAGCTCATTC | 1645 |
| Core 2 Side3 5 | 21[520] | 9[519] | AATCAGAGCCTAACGTGCTTTCCTTCATTG | 1646 |
| Core 2 Side3 6 | 11[544] | 0[544] | AAAGGAGCGGGCGCTAACCCTAAAGGGAGC | 1647 |
| Core 2 Side3 7 | 17[544] | 16[560] | AACAAAGTACAACGGAGATTCTATGCATCAG | 1648 |
| Core 2 Side3 8 | 17[504] | 6[520] | ACACTCATCTTTGACCCCCAGCGACAGGTAG | 1649 |
| Core 2 Side3 9 | 7[544] | 8[560] | ACCTGCTCCATGTTACTTAGAAGGGGAAGAA | 1650 |
| Core 2 Side3 10 | 15[512] | 9[527] | AGGCACCAACCTCTCAGTTTTGCAATCCCCC | 1651 |
| Core 2 Side3 11 | 0[583] | 10[568] | GGTGCCGTAAAGCACTGGACTCCGTTTTTCT | 1652 |
| Core 2 Side3 12 | 13[560] | 12[544] | ACATACGATGTAGCGGTCACGCTGCGCGTAA | 1653 |
| Core 2 Side3 13 | 4[606] | 3[591] | TCCAATAAATCATACAGGCAAGGAAGGCTTG | 1654 |
| Core 2 Side3 14 | 8[519] | 16[504] | CAGAGGGGGTAATAGCCAAAATAGCAAAAGA | 1655 |
| Core 2 Side3 15 | 16[503] | 8[488] | ATACAGATAAATAAGGGTAAAATATTAGACTG | 1656 |
| Core 2 Side3 16 | 0[543] | 10[528] | CCCCGATTATCCTGTTGCTGATTGCCCTTAAT | 1657 |
| Core 2 Side3 17 | 1[456] | 11[471] | GGTCAGGATTAGAGAGCGAAAGACAGTTTCAG | 1658 |
| Core 2 Side3 18 | 17[488] | 7[503] | ACCGACCGGGAATACCCAGACGACGATAAAAA | 1659 |
| Core 2 Side3 19 | 2[608] | 1[591] | CTTGAGATGGTTTAATTTCAACTTTAAGGGCGA | 1660 |
| Core 2 Side3 20 | 22[559] | 9[551] | ACGAACCACCAGCAGTCACAATCGTAATCTGAGAGA | 1661 |
| Core 2 Side3 21 | 14[559] | 1[551] | CTCGAATTTCCACACAGTGAGACGGGCAACACAAGAGT | 1662 |
| Core 2 Side3 22 | 13[504] | 20[512] | TTGCTTTGAACACCGCTCTGAATATCGTTAGGGAATTA | 1663 |
| Core 2 Side3 23 | 21[528] | 13[519] | CGGGAGCTACAGAGGTGAGGCGGTCAGTATTACGAGCA | 1664 |
| Core 2 Side3 24 | 11[512] | 14[504] | GAACGTGCAGTTCAGAAAACGACATAAATAATGGAAGG | 1665 |
| Core 2 Side3 25 | 9[480] | 13[503] | ACCGTCCAATACTGCGGAATCGTGAATGACCTTGTATGG | 1666 |
| Core 2 Side3 26 | 13[520] | 12[512] | CGTATGTGTGAAATTGCCCGCCGCGCTTAATGCGCCGCT | 1667 |
| Core 2 Side3 27 | 15[504] | 19[527] | CACTACGATCATCATACTAACAACTAATAGATTAGAGCC | 1668 |
| Core 2 Side3 28 | 17[528] | 20[520] | ATTATACCGCGCCTGTGTCAATAGAACCACCAGAAGGAGC | 1669 |
| Core 2 Side3 29 | 7[520] | 14[528] | GCTTTTGCAAAGAACATAACGAGATGTGGTGCAGCTGTTT | 1670 |
| Core 2 Side3 30 | 5[496] | 17[503] | ACGAGTAGATTTAGTTCATCAGTTGAGATTTATGTCTAAA | 1671 |
| Core 2 Side3 31 | 4[575] | 6[552] | TTAGCAAATTGGGGCGAGAGCATAAAGTGTATCATCGCCT | 1672 |
| Core 2 Side3 32 | 0[511] | 12[504] | AAGCAAAGCGGATTGCTGACTATTATAGTCAGACAGGGCG | 1673 |
| Core 2 Side3 33 | 22[543] | 16[544] | AAGATAAAAAACAGGAGGCCGATTTTGCGGGAATGCGGCGCAG | |
| | | | T | 1674 |
| Core 2 Side3 34 | 6[551] | 4[552] | GATAAATAACCTGTTTAGCTATATTTTCATATTCCGGATAGGCTG | |
| | | | G | 1675 |
| Core 2 Side3 35 | 14[495] | 9[479] | CTACCATATGATTGCTATAAATCAAAAATATAGAAAGGAAATATG | 1676 |
| | | | CA | |
| Core 2 Side3 36 | 9[528] | 19[543] | TCACACCGCCTGGCCCATGGTCATTGCGGCCAAACAAAGAATAA | 1677 |
| | | | TACA | |
| Core 2 Side3 37 | 16[559] | 10[560] | ACGATCCAGCGGGCCGAAATCTACGTTTAAGAACTGGCTCTTTC | 1678 |
| | | | ACCA | |
| Core 2 Side3 38 | 10[527] | 13[543] | GCTTTAAAGCGAGAAAGGAAGGGAAGAAAGCGCCACCACATTAT | 1679 |
| | | | CCGC | |
| Core 2 Side3 39 | 8[503] | 22[486] | TAAAATGTCGTAATGCGTTAGAACATTATACTCTGCAACAGTGCC | 1680 |
| | | | ACGCT | |

TABLE 14
Core 1 Side 12; Side 1-2 Connector

| | | | | SEQ ID |
|---|---|---|---|---|
| | Start | End | Sequence | NO: |
| Core1 Side12 1 | 2[192] | 3[271] | AAACAGCGGTCTCCGTTTTTTGGTGAAGGGATAGCT | 1681 |
| Core1 Side12 2 | 6[279] | 5[167] | TAAGATTTTTACGCGAGGCGTTGTTGGGA | 1682 |
| Core1 Side12 3 | 19[136] | 19[311] | GCAACAGGAAAAATTTTTCGCTCATGGAAAT | 1683 |
| Core1 Side12 4 | 6[71] | 5[87] | TCTCCGTTGAGCGAGTAACAACCCACTCCAG | 1684 |
| Core1 Side12 5 | 14[287] | 14[144] | ATAGCAGTTTTTCCTTTACAGAGAGAATAACATAA | 1685 |
| Core1 Side12 6 | 1[184] | 2[255] | TTTGCCGCCAGTTTTTCAGTTGGGGGGGAGACGCAG | 1686 |
| Core1 Side12 7 | 12[287] | 12[152] | CCCACGCAACCATTTTTGCTTACGGCTGGAGGTGTCC | 1687 |
| Core1 Side12 8 | 20[311] | 20[136] | TCTTTGATTAGTAATATTTTTACATCACTTGCCTGAG | 1688 |
| Core1 Side12 9 | 11[160] | 11[271] | TGGTCTGGTCAGCAGCAACCGCAATTTTTGAATGCCAA | 1689 |
| Core1 Side12 10 | 5[168] | 5[279] | AAGCCGTTTTTATTTTCATTTTTTCGTAGGAATCATTACC | 1690 |
| Core1 Side12 11 | 21[136] | 14[296] | CTGTCCATCACGCAAATTTTTTTAACCGTTGTAACGTCAAA | 1691 |
| Core1 Side12 12 | 4[279] | 4[168] | CGGGTATTAAACCAAGTACTTTTTCGCACTCATCGAGAACAAGC | 1692 |
| Core1 Side12 13 | 10[151] | 13[287] | AAGGTAAAGCTAATATCAGAGAGATAACCCACATTTTTAGAATTG | 1693 |
| | | | AG | |
| Core1 Side12 14 | 5[88] | 19[87] | CCAGCTTTGGCCTCAGGAAGATCGATACTTTTTCAGATGCACAAT | 1694 |
| | | | TCG | |
| Core1 Side12 15 | 7[168] | 5[295] | TATTCACTTTTTAAACAAATAAATCCTCATTAAAGCTTATCTAGCA | 1695 |
| | | | AGC | |
| Core1 Side12 16 | 0[271] | 0[176] | ACATAAAAAAATCCCGTAAAAATTTTTAAGCCGCACAGGCGGCCT | 1696 |
| | | | TTAG | |
| Core1 Side12 17 | 16[295] | 17[303] | GTTGCTATTTTTTTTTGCACCCAGGACTTGCGTTTTTGGAGGTTT | 1697 |
| | | | TGAAGCC | |
| Core1 Side12 18 | 7[144] | 15[295] | GCAGGTCACCACCCTCAGCCATATTATTTATcCCAATTTTTTCCA | 1698 |
| | | | AATAAGAAA | |

TABLE 15
Core 2 Side 23; Side 2-3 Connector

| | | | | SEQ ID |
|---|---|---|---|---|
| | Start | End | Sequence | NO: |
| Core2 Side23 1 | 15[376] | 15[487] | TTAATTGAGGAAGTTTTTTTTCCATTAAACGG | 1699 |
| Core2 Side23 2 | 14[487] | 14[376] | TCAAAATTATTTGTTTTTTACGTAAAACAGAAA | 1700 |
| Core2 Side23 3 | 13[376] | 13[487] | ACATCGGGAGAAACTTTTTTATAACGGATTCGCC | 1701 |
| Core2 Side23 4 | 9[384] | 2[456] | ATAATATAATGCTGTAGCTTTTTTCAACCAAACTAC | 1702 |
| Core2 Side23 5 | 19[360] | 19[503] | GAGGAAGGTTATCTAATTTTTTTTATCTTTAGGAGCA | 1703 |
| Core2 Side23 6 | 11[472] | 12[376] | CGGAGTGACGCGCAGAGTTTTTTCGAATTATTCATTT | 1704 |
| Core2 Side23 7 | 21[352] | 21[495] | ATATCAAACCCTCAATATAATCCTTTTTTTATTGTTTGG | 1705 |
| Core2 Side23 8 | 5[408] | 4[464] | GCCTATTTTTTTTTTGAACCTATTATTCTGAATATAATTCCAAC | 1706 |
| Core2 Side23 9 | 10[463] | 11[463] | AGGAATTTTTTTGCGAATAATATGCTAAACAACTTTTTTTTCAAC | 1707 |
| Core2 Side23 10 | 6[375] | 17[487] | TGTCCAGATCATCTTCTGACCTAAATTTAATTTTTTGGTTTGAAAT | 1708 |
| Core2 Side23 11 | 20[503] | 20[352] | TTCCTGATTATCAGATTTTTTTTTGGCAATTCATCAATCAATATCT | 1709 |
| Core2 Side23 12 | 8[487] | 8[384] | GATAGTAAAGTATTTTGCGGATGGCTTTTTTTAGAGCTTAATTGC | 1710 |
| | | | TGGA | |
| Core2 Side23 13 | 0[463] | 0[392] | TTCAAATATCGCGTTTTAATTTTTTTCGAGCTTCAAAGCGTAAATG | 1711 |
| | | | AATTT | |
| Core2 Side23 14 | 4[463] | 3[399] | ATGAAAGTATTAAGATTTTTTTCTGAGACTCCTCAAGAGAAGGAT | 1712 |
| | | | TAGCCCAAT | |
| Core2 Side23 15 | 16[487] | 15[367] | CGTTAAATAAGTTTTTTATAAACACCGGAATCATAATTACTAGAAA | 1713 |
| | | | ACGCTCAAC | |
| Core2 Side23 16 | 10[391] | 1[455] | ATTTTTTCGTCTTTCCAGACGTTAGAACCAGACCGGAAGTTTTTT | 1714 |
| | | | TAACTCCAACA | |
| Core2 Side23 17 | 7[368] | 7[479] | AAAGTACCGACAAAAGGTCGAGGCATAGTAAGAGTTTTTTAACAC | 1715 |
| | | | TATCATAACCCT | |

TABLE 16
Core 2 Side 31; Side 3-1 Connector

| | | | | SEQ ID |
|---|---|---|---|---|
| | Start | End | Sequence | NO: |
| Core2 Side31 1 | 14[71] | 14[568] | GAGCCTCCTCACATTTTTTTTGAGGATCCCCGG | 1716 |
| Core2 Side31 2 | 22[87] | 22[560] | CCTAAAACATCGCCATTTTTTTTAAAATACCGA | 1717 |
| Core2 Side31 3 | 19[544] | 19[79] | TTTGAGGATTTAGAAGTATTATTTTTTTCTTTACAA | 1718 |
| Core2 Side31 4 | 16[79] | 16[568] | CGGGTTACCTGCAGCCAGCGGTTTTTTGCCGGTGCCCCC | 1719 |
| Core2 Side31 5 | 15[568] | 15[79] | GTCATACCGGGGGTTTTTTTTCTGCCAGCACGCGTGCCTG | 1720 |
| Core2 Side31 6 | 12[575] | 13[71] | GCCCGAAGGCCGGAAGCATAAAGTTTTTTGTAAAGCCTGGGG | 1721 |
| Core2 Side31 7 | 12[87] | 12[576] | AGCCGTGAGCTAACTCACATTAATTGCGTTTTTTTGCGCTCACT | 1722 |
| Core2 Side31 8 | 20[87] | 21[559] | TTATTAATTTTAAAAGTTTTTTTTGAGTAACATTATCATTAAAGGGA | 1723 |
| Core2 Side31 9 | 9[568] | 2[31] | ATTATACCAGCTATCAGGTCATTGCCTAGAACGCCATCAGTAAATT | 1724 |
| | | | G | |
| Core2 Side31 10 | 15[560] | 21[87] | TTTTCACGGTACCGAGTTTTAGACATTTTTTTAACGGTACGCCAGAA | 1725 |
| | | | T | |
| Core2 Side31 11 | 5[576] | 5[47] | CGAGCTGAAAAGGTGGCATCAATTCTACTTTTTTTATAGTAGTCCA | 1726 |
| | | | GCTTT | |
| Core2 Side31 12 | 8[575] | 4[32] | AGGACGTTCAATAAAGCCGGTCACGTTGGTGTGATTCCTGTAGAG | 1727 |
| | | | CATTAA | |
| Core2 Side31 13 | 10[71] | 10[576] | CCCCAAACGCGCGGGGAGAGGCGGTTTTTTTTGCGTATTGGGCGC | 1728 |
| | | | CAGGGTG | |
| Core2 Side31 14 | 11[576] | 11[71] | CAGTCGGGAAACCTGTCGTGCTTTTTTAGCTGCATTAATGAATCGG | 1728 |
| | | | CCAAAAC | |
| Core2 Side31 15 | 17[568] | 7[79] | AATCGGTTGTATTTTTTCAAAAACATTATGACCCTGTACGTCGGATG | 1730 |
| | | | CCAGTTT | |
| Core2 Side31 16 | 5[48] | 6[576] | CATCAACATTAAATGGGGAACAAACGGCGGATTGTTTTTTCCGTAA | 1731 |
| | | | TGGGATATC | |
| Core2 Side31 17 | 8[63] | 4[576] | GGAGAGGGTAGCTATTTTTTTTTTTAGAGATCTACAAAGGTCAGTG | 1732 |
| | | | AATCAAAGAA | |
| Core2 Side31 18 | 0[55] | 0[584] | ATTTTTGTTAAATCAACGTGAACCTTTTTTTCACCCAAATCAAGTTTT | 1733 |
| | | | TTGGGGTCGA | |

TABLE 17
Active Hubble1; Side 1 Hubble

| | | | | SEQ ID |
|---|---|---|---|---|
| | Start | End | Sequence | NO: |
| Active Hubble1 1 | 30[167] | 2[152] | ACTGTAGCCGTTTGCCTTGCCTTTATAGCCCC | 1734 |
| Active Hubble1 2 | 32[167] | 5[159] | GGGGATGTGCTGCAAGCGCCAGCTTCGGTGCGGCGCAACT | 1735 |
| Active Hubble1 3 | 33[136] | 2[144] | ACGCCAGGCGCTATTAGCGATTAACCATGTTTGCCTCCCT | 1736 |
| Active Hubble1 4 | 2[143] | 30[115] | CAGAGCAAAGCCACCAATAATCAAAATCACCGCAATGAAACCATC | 1737 |
| Active Hubble1 5 | 3[168] | 31[167] | TGTGAGAGATAGACTTATCAAACTTAAGCATTTTCGGTCAGCGTCA | 1738 |
| | | | G | |
| Active Hubble1 6 | 31[115] | 15[119] | GATAGCAGCACCGAGTAGCACAACAATCGACCACCACCAGAGAAT | 1739 |
| | | | CAGAGCCT | |
| Active Hubble1 7 | 8[279] | 33[167] | TAACGGAATATTTTTCCCAAAAGAACTGGCCTCGGAATTAGGGCGA | 1740 |
| | | | GGCGAAAG | |
| Active Hubble1 8 | 6[159] | 32[136] | TTAGCGAACCTAAATGCAATGCCTAGGTTGAGGTTTTCCCGTACAG | 1741 |
| | | | CGGTTGGGTA | |

TABLE 18
Active Hole1; Side 1 Hole

| | | | | SEQ ID |
|---|---|---|---|---|
| | Start | End | Sequence | NO: |
| Active Hole1 1 | 4[55] | 7[71] | CTGGCCTGGGCGCATCGTAACCGTGCATCT | 1742 |
| Active Hole1 2 | 3[88] | 4[88] | GAGCCGCCACGGGAACCAAGCTTTCAGAGGTG | 1743 |
| Active Hole 1 3 | 4[111] | 2[109] | GCCAGTGCGGATAACCTCACCGGACATTACCATTA | 1744 |
| Active Hole 1 4 | 2[55] | 8[64] | AATAGGAGTCTGGAGCAAACAAGAGAATCGTAATGCC | 1745 |
| Active Hole1 5 | 1[109] | 12[120] | GCAAGGCCGGATTTTTTCGATCCTCATAACGGAACCGCTTTCG | 1746 |
| Active Hole1 6 | 3[592] | 3[55] | CCCTGACGAGAAACACTTTTTTTGAACGAGTAAAAATAATTCGCG | 1747 |
| | | | T | |
| Active Hole1 7 | 1[592] | 1[55] | AAAACCGTCTATCAGGGCTTTTTTTTGGCCCACTGCTCATTTTTTA | 1748 |
| | | | ACC | |

TABLE 19
Active Hubble2; Side 2 Hubble

| | | | | SEQ ID |
|---|---|---|---|---|
| | Start | End | Sequence | NO: |
| Active Hubble2 1 | 32[370] | 31[375] | CCCTCAGACGTTATTCGGTCGCTGAGG | 1749 |
| Active Hubble2 2 | 31[339] | 2[333] | ATCGCCCACGCATAATTTCTTAAACAGCTTGA | 1750 |
| Active Hubble2 3 | 1[360] | 30[339] | GAACGAGGCTCAGCAGCGAAAGACAATGACAACAACC | 1751 |
| Active Hubble2 4 | 30[391] | 3[383] | GGCCGCTTTTGCGGGACTTGCAGGCGATCTAATTTTCAGG | 1752 |
| Active Hubble2 5 | 1[352] | 32[339] | AGCATCGCGAGGTGAACCGATAACTCAGGAGGTTTAGTACCGC | 1753 |
| Active Hubble2 6 | 33[339] | 6[336] | CACCCTCAGAACCTGCCGTCGATAAGTTTACATGTTCAGCTAATG | 1754 |
| Active Hubble2 7 | 9[352] | 33[370] | ATCAGCTACATGTAGGTGTATGGATAAGGCCACCCTCAGAACCG | 1755 |
| | | | CCA | |
| Active Hubble2 8 | 2[455] | 31[391] | AACGCCTGTTTTTTTTCATTCCACAGACAGCCCTCATAGTTAGCG | 1756 |
| | | | TAAGAGTTAAA | |

TABLE 20
Active Hole2; Side 2 Hole

| | | | | SEQ ID |
|---|---|---|---|---|
| | Start | End | Sequence | NO: |
| Active Hole2 1 | 3[272] | 3[300] | CTAAACGCAACAATCAATAATCGGCTGT | 1757 |
| Active Hole2 2 | 5[320] | 4[333] | TGATGATACAGGAGTGTACTGGTAAGAGGGTTGAT | 1758 |
| Active Hole2 3 | 1[333] | 11[351] | TACCGATAGTTATAACCTTAGAAAACAAAATTAAT | 1759 |
| Active Hole2 4 | 3[333] | 8[336] | ATAAGTATAGCCCGGAATAATTTAGGCAGAGGAAG | 1760 |
| Active Hole2 5 | 4[300] | 6[280] | CTTTCCTTATCATTCCAAGAAGCGCCCAACGGTATTC | 1761 |
| Active Hole2 6 | 2[279] | 10[168] | CGAGGCAGCAGTATGGCGCCAAAGACAAATTTTTAGGGCGACATT | 1762 |
| | | | CAA | |
| Active Hole2 7 | 9[160] | 1[279] | CCATGATTAAGACTCCTTATTTTTTTACCGGAAAAATTGTGTACATG | 1763 |
| | | | AAAC | |

TABLE 21
Active Hubble3; Side 3 Hubble

| | | | | SEQ ID |
|---|---|---|---|---|
| | Start | End | Sequence | NO: |
| Active Hubble3 1 | 31[531] | 2[525] | GAAATCGGCAAAAGTCCACGCTGGTTTGCCCC | 1764 |
| Active Hubble3 2 | 2[551] | 32[531] | GCAAGCGTCCCTTATACTGACCAACTTTGAAAGAGG | 1765 |
| Active Hubble3 3 | 32[572] | 2[552] | GGTCAATCATAAGACAAAGCTCCTTATGCGATTTGCA | 1766 |
| Active Hubble3 4 | 1[552] | 30[531] | CCACTATTTTGTTCCAGTTTGGAATGATGGTGGTTCC | 1767 |
| Active Hubble3 5 | 3[552] | 31[572] | TCAACGTAGGAACCGAAAATCAAAAGAATAGCCCGAG | 1768 |
| Active Hubble3 6 | 30[572] | 11[575] | ATAGGGTTGAGTGAAAGAACGTAAATCGGAGGGCGCTGGCCTTTC | 1769 |
| Active Hubble3 7 | 33[531] | 6[528] | ACAGATGAACGGTTCATCAAGTGGTCAATTGTGTCGAACATTATT | 1770 |
| Active Hubble3 8 | 8[551] | 33[572] | GTTAATAACCCAAACTGACCTGTACAGACCAGGCGCAACGAGGCG | 1771 |
| | | | CAGAC | |

TABLE 22
Active Hole3; Side 3 Hole

| | | | | SEQ ID |
|---|---|---|---|---|
| | Start | End | Sequence | NO: |
| Active Hole3 1 | 2[482] | 10[464] | TGCTCCTTTTGATAAGTAATGTTTTAACAACTAA | 1772 |
| Active Hole3 2 | 5[512] | 4[525] | TGACCATTAGATACATTTCGCAAAAGTAATCTTGA | 1773 |
| Active Hole3 3 | 1[525] | 0[512] | AGCAGGCGAAATAGAGCTTGACGGGGAAAGCCGGC | 1774 |
| Active Hole3 4 | 11[488] | 1[482] | CTTTACCCATCAAAAAGATTAAGAGGAAGCCTACCTTTAAT | 1775 |
| Active Hole3 5 | 4[482] | 6[392] | TGGAAGTTTCACAGTTGAACTAATGCAGATACATTTTTTTACGCCAA | 1776 |
| | | | AAGG | |
| Active Hole3 6 | 3[525] | 17[543] | CAAGAACCGGATATTCATTAAACGAACAATCCGCGGTCACTGCAAG | 1777 |
| | | | CGCGA | |
| Active Hole3 7 | 3[400] | 3[482] | AGGAACCCATGTACCGTTTTTTTACACTGAGTTTCGTCACCAGAGG | 1778 |
| | | | TCATCGGTGTC | |

TABLE 23
Passive Hubble1; Side 1 Hubble

| | | | | SEQ |
|---|---|---|---|---|
| | | | | ID |
| | Start | End | Sequence | NO: |
| Passive | 30[167] | 2[152] | TTTTTACTGTAGCCGTTTGCCTTGCCTTTATAGCCCC | 1779 |
| Hubble1 1 | | | | |
| Passive | 32[167] | 5[159] | TTTTTGGGGATGTGCTGCAAGCGCCAGCTTCGGTGCGGCGCAACT | 1780 |
| Hubble1 2 | | | | |
| Passive | 33[136] | 2[144] | TTTTTACGCCAGGCGCTATTAGCGATTAACCATGTTTGCCTCCCT | 1781 |
| Hubble1 3 | | | | |
| Passive | 2[143] | 30[115] | CAGAGCAAAGCCACCAATAATCAAAATCACCGCAATGAAACCATCT | 1782 |
| Hubble1 4 | | | TTTT | |
| Passive | 3[168] | 31[167] | TGTGAGAGATAGACTTATCAAACTTAAGCATTTTCGGTCAGCGTCA | 1783 |
| Hubble1 5 | | | GTTTTT | |
| Passive | 31[115] | 15[119] | TTTTTGATAGCAGCACCGAGTAGCACAACAATCGACCACCACCAGA | 1784 |
| Hubble1 6 | | | GAATCAGAGCCT | |
| Passive | 8[279] | 33[167] | TAACGGAATATTTTTCCCAAAAGAACTGGCCTCGGAATTAGGGCGA | 1785 |
| Hubble1 7 | | | GGCGAAAGTTTTT | |
| Passive | 6[159] | 32[136] | TTAGCGAACCTAAATGCAATGCCTAGGTTGAGGTTTTCCCGTACAG | 1786 |
| Hubble1 8 | | | CGGTTGGGTATTTTT | |

TABLE 24
Passive Hole1; Side 1 Hole

| | | | | SEQ ID |
|---|---|---|---|---|
| | Start | End | Sequence | NO: |
| Passive Hole1 1 | 4[55] | 7[71] | TTTTTCTGGCCTGGGCGCATCGTAACCGTGCATCT | 1787 |
| Passive Hole1 2 | 3[88] | 4[88] | TTTTTGAGCCGCCACGGGAACCAAGCTTTCAGAGGTGTTTTT | 1788 |
| Passive Hole1 3 | 4[111] | 2[109] | GCCAGTGCGGATAACCTCACCGGACATTACCATTATTTTT | 1789 |
| Passive Hole1 4 | 2[55] | 8[64] | TTTTTAATAGGAGTCTGGAGCAAACAAGAGAATCGTAATGCC | 1790 |
| Passive Hole1 5 | 1[109] | 12[120] | TTTTTGCAAGGCCGGATTTTTTCGATCCTCATAACGGAACCGCTTT | 1791 |
| | | | CG | |
| Passive Hole1 6 | 3[592] | 3[55] | CCCTGACGAGAAACACTTTTTTTGAACGAGTAAAAATAATTCGCGTT | 1792 |
| | | | TTTT | |
| Passive Hole1 7 | 1[592] | 1[55] | AAAACCGTCTATCAGGGCTTTTTTTTGGCCCACTGCTCATTTTTTAA | 1793 |
| | | | CCTTTTT | |

TABLE 25
Passive Hubble2; Side 2 Hubble

| | | | | SEQ ID |
|---|---|---|---|---|
| | Start | End | Sequence | NO: |
| Passive | 32[370] | 31[375] | TTTTTCCCTCAGACGTTATTCGGTCGCTGAGG | 1794 |
| Hubble2 1 | | | | |
| Passive | 31[339] | 2[333] | TTTTTATCGCCCACGCATAATTTCTTAAACAGCTTGATTTTT | 1795 |
| Hubble2 2 | | | | |
| Passive | 1[360] | 30[339] | GAACGAGGCTCAGCAGCGAAAGACAATGACAACAACCTTTTT | 1796 |
| Hubble2 3 | | | | |
| Passive | 30[391] | 3[383] | TTTTTGGCCGCTTTTGCGGGACTTGCAGGCGATCTAATTTTCAGG | 1797 |
| Hubble2 4 | | | | |
| Passive | 1[352] | 32[339] | AGCATCGCGAGGTGAACCGATAACTCAGGAGGTTTAGTACCGCTT | 1798 |
| Hubble2 5 | | | TTT | |
| Passive | 33[339] | 6[336] | TTTTTCACCCTCAGAACCTGCCGTCGATAAGTTTACATGTTCAGCTA | 1799 |
| Hubble2 6 | | | ATG | |
| Passive | 9[352] | 33[370] | ATCAGCTACATGTAGGTGTATGGATAAGGCCACCCTCAGAACCGC | 1800 |
| Hubble2 7 | | | CATTTTT | |
| Passive | 2[455] | 31[391] | AACGCCTGTTTTTTTTCATTCCACAGACAGCCCTCATAGTTAGCGTA | 1801 |
| Hubble2 8 | | | AGAGTTAAATTTTT | |

TABLE 26
Passive Hole2; Side 2 Hole

| | | | | SEQ ID |
|---|---|---|---|---|
| | Start | End | Sequence | NO: |
| Passive Hole2 1 | 3[272] | 3[300] | CTAAACGCAACAATCAATAATCGGCTGTTTTTT | 1802 |
| Passive Hole2 2 | 5[320] | 4[333] | TGATGATACAGGAGTGTACTGGTAAGAGGGTTGATTTTTT | 1803 |
| Passive Hole2 3 | 1[333] | 11[351] | TTTTTTACCGATAGTTATAACCTTAGAAAACAAAATTAAT | 1804 |
| Passive Hole2 4 | 3[333] | 8[336] | TTTTTATAAGTATAGCCCGGAATAATTTAGGCAGAGGAAG | 1805 |
| Passive Hole2 5 | 4[300] | 6[280] | TTTTTCTTTCCTTATCATTCCAAGAAGCGCCCAACGGTATTC | 1806 |
| Passive Hole2 6 | 2[279] | 10[168] | TTTTTCGAGGCAGCAGTATGGCGCCAAAGACAAATTTTTAGGGCGA | 1807 |
| | | | CATTCAA | |
| Passive Hole2 7 | 9[160] | 1[279] | CCATGATTAAGACTCCTTATTTTTTTACCGGAAAAATTGTGTACATG | 1808 |
| | | | AAACTTTTT | |

TABLE 27
Passive Hubble3; Side 3 Hubble

| | | | | SEQ ID |
|---|---|---|---|---|
| | Start | End | Sequence | NO: |
| Passive | 31[531] | 2[525] | TTTTTGAAATCGGCAAAAGTCCACGCTGGTTTGCCCCTTTTT | 1809 |
| Hubble3 1 | | | | |
| Passive | 2[551] | 32[531] | GCAAGCGTCCCTTATACTGACCAACTTTGAAAGAGGTTTTT | 1810 |
| Hubble3 2 | | | | |
| Passive | 32[572] | 2[552] | TTTTTGGTCAATCATAAGACAAAGCTCCTTATGCGATTTGCA | 1811 |
| Hubble3 3 | | | | |
| Passive | 1[552] | 30[531] | CCACTATTTTGTTCCAGTTTGGAATGATGGTGGTTCCTTTTT | 1812 |
| Hubble3 4 | | | | |
| Passive | 3[552] | 31[572] | TCAACGTAGGAACCGAAAATCAAAAGAATAGCCCGAGTTTTT | 1813 |
| Hubble3 5 | | | | |
| Passive | 30[572] | 11 [575] | TTTTTATAGGGTTGAGTGAAAGAACGTAAATCGGAGGGCGCTGGC | 1814 |
| Hubble3 6 | | | CTTTC | |
| Passive | 33[531] | 6[528] | TTTTTACAGATGAACGGTTCATCAAGTGGTCAATTGTGTCGAACATT | 1815 |
| Hubble3 7 | | | ATT | |
| Passive | 8[551] | 33[572] | GTTAATAACCCAAACTGACCTGTACAGACCAGGCGCAACGAGGCG | 1816 |
| Hubble3 8 | | | CAGACTTTTT | |

TABLE 28
Passive Hole3; Side 3 Hole

| | | | | SEQ ID |
|---|---|---|---|---|
| | Start | End | Sequence | NO: |
| Passive Hole3 1 | 2[482] | 10[464] | TTTTTTGCTCCTTTTGATAAGTAATGTTTTAACAACTAA | 1817 |
| Passive Hole3 2 | 5[512] | 4[525] | TGACCATTAGATACATTTCGCAAAAGTAATCTTGATTTTT | 1818 |
| Passive Hole3 3 | 1[525] | 0[512] | TTTTTAGCAGGCGAAATAGAGCTTGACGGGGAAAGCCGGC | 1819 |
| Passive Hole3 4 | 11[488] | 1[482] | CTTTACCCATCAAAAAGATTAAGAGGAAGCCTACCTTTAATTTTTT | 1820 |
| Passive Hole3 5 | 4[482] | 6[392] | TTTTTTGGAAGTTTCACAGTTGAACTAATGCAGATACATTTTTTTAC | 1821 |
| | | | GCCAAAAGG | |
| Passive Hole3 6 | 3[525] | 17[543] | TTTTTCAAGAACCGGATATTCATTAAACGAACAATCCGCGGTCACT | 1822 |
| | | | GCAAGCGCGA | |
| Passive Hole3 7 | 3[400] | 3[482] | AGGAACCCATGTACCGTTTTTTTACACTGAGTTTCGTCACCAGAGG | 1823 |
| | | | TCATCGGTGTCTTTTT | |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A three-dimensional DNA molecular structure comprising:
a DNA strand folded in the form of a nanoscale triangular subunit having a configuration that allows a plurality of said nanoscale triangular subunits to self-assemble in the form of a macromolecular cylindrical shell.

2. The three-dimensional DNA molecular structure according to claim 1, wherein the plurality of said nanoscale triangular subunits self-assemble by lateral edge-to-edge stacking via base-pair stacking.

3. The three-dimensional DNA molecular structure according to claim 2, wherein each of the three edges of the nanoscale triangular subunits mate with only one of the other two edges.

4. The three-dimensional DNA molecular structure according to any one of claims 1 to 3, wherein the three sides of the nanoscale triangular subunit comprise bevel angles of about 10.4°, about 10.4°, and about −5.3°.

5. The three-dimensional DNA molecular structure according to claim 1, wherein one side of the nanoscale triangular subunit has a different bevel angle from the other two sides, which causes misalignment at an associated vertex, and the three-dimensional DNA molecular structure further comprises an additional ss-DNA molecule self-assembled into the nanoscale triangular subunit along the one side.

6. The three-dimensional DNA molecular structure according to claim 5, wherein the additional ss-DNA molecule is positioned along a base surface of the nanoscale triangular subunit.

7. The three-dimensional DNA molecular structure according to claim 1, further comprising a targeting moiety linked to the nanoscale triangular subunit along a base surface.

8. The three-dimensional DNA molecular structure according to claim 7, wherein the targeting moiety is an antibody, active antibody fragment, nucleic acid aptamer, or peptide antibody mimic.

9. The three-dimensional DNA molecular structure according to claim 7, wherein the targeting moiety binds to a viral capsid protein.

10-11. (canceled)

12. A macromolecular cylindrical shell formed by self-assembly of a plurality of the three-dimensional DNA molecular structures according to claim 1.

13. The macromolecular cylindrical shell according to claim 12, wherein the three-dimensional DNA molecular structures are self-assembled by lateral edge-to-edge stacking via base-pair stacking, and the macromolecular cylindrical shell further comprises a linking agent that binds to two edge-to-edge stacked nanoscale triangular subunits.

14. (canceled)

15. The macromolecular cylindrical shell according to claim 12, wherein the cylindrical shell has a 5,0 lattice structure, a 5,3 lattice structure, or a 5,5 lattice structure.

16. The macromolecular cylindrical shell according to claim 16, wherein the cylindrical shell is configured to encapsulate a filamentous virus particle.

17. A composition comprising a plurality of three-dimensional DNA molecular structures according to claim 1 in a carrier.

18. (canceled)

19. A composition comprising a plurality of three-dimensional DNA molecular structures according to claim 1 and a plurality of macromolecular cylindrical shells assembled from the plurality of three-dimensional DNA molecular structures in a carrier.

20. (canceled)

21. The composition according to claim 17, wherein the carrier is a pharmaceutically acceptable carrier.

22-23. (canceled)

24. A method of encapsulating a filamentous viral particle comprising:
providing a plurality of the three-dimensional DNA molecular structures according to claim 1, and allowing said three-dimensional DNA molecular structures to self-assemble around a filamentous viral particle to form a cylindrical shell, thereby encapsulating the filamentous viral particle.

25. A method of inhibiting viral infection comprising:
encapsulating a filamentous viral particle with a macromolecular cylindrical shell according to claim 12, whereby the macromolecular cylindrical shell forms a physical barrier to inhibit filamentous viral particle infection of a cell otherwise susceptible to infection by the filamentous viral particle.

26-30. (canceled)

31. A method of treating an individual for a viral infection, the method comprising:
administering a composition according to claim 21 to an individual at a site of viral infection, wherein the macromolecular cylindrical shell forms a physical barrier that encapsulates filamentous viral particles at the site of viral infection, thereby treating the individual.

32. The method according to claim 31, wherein said administering is by oral, mucosal, topical, or systemic delivery.

33. (canceled)
